SEQUENCE LISTING (43 sequences)

SEQ ID NO: 1 - DNA, 26 bp, synthetic construct
  source 1..26: mol_type = other DNA
acaggctaacgaacgatctcgagcgc

SEQ ID NO: 2 - DNA, 26 bp, synthetic construct
  source 1..26: mol_type = other DNA
gcgctcgagatcgttcgttagcctgt

SEQ ID NO: 3 - DNA, 26 bp, synthetic construct
  source 1..26: mol_type = other DNA
cgtagtgagacttacagcttcgtagg

SEQ ID NO: 4 - DNA, 26 bp, synthetic construct
  source 1..26: mol_type = other DNA
cctacgaagctgtaagtctcactacg

SEQ ID NO: 5 - DNA, 26 bp, synthetic construct
  source 1..26: mol_type = other DNA
  misc_feature 26: note = 3 prime amino modification
acaggctaacgaacgatctcgagcgc

SEQ ID NO: 6 - DNA, 26 bp, synthetic construct
  source 1..26: mol_type = other DNA
  misc_feature 1: note = 5 prime amino modification
gcgctcgagatcgttcgttagcctgt

SEQ ID NO: 7 - DNA, 26 bp, synthetic construct
  source 1..26: mol_type = other DNA
  misc_feature 1: note = 5 prime amino modification
acaggctaacgaacgatctcgagcgc

SEQ ID NO: 8 - DNA, 26 bp, synthetic construct
  source 1..26: mol_type = other DNA
  misc_feature 26: note = 3 prime amino modification
cgtagtgagacttacagcttcgtagg

SEQ ID NO: 9 - DNA, 52 bp, synthetic construct
  source 1..52: mol_type = other DNA
acaggctaacgaacgatctcgagcgccctacgaagctgtaagtctcactacg

SEQ ID NO: 10 - DNA, 53 bp, synthetic construct
  source 1..53: mol_type = other DNA
acaggctaacgaacgatctcgagcgctcctacgaagctgtaagtctcactacg

SEQ ID NO: 11 - DNA, 54 bp, synthetic construct
  source 1..54: mol_type = other DNA
acaggctaacgaacgatctcgagcgcttcctacgaagctgtaagtctcactacg

SEQ ID NO: 12 - DNA, 55 bp, synthetic construct
  source 1..55: mol_type = other DNA
acaggctaacgaacgatctcgagcgctttcctacgaagctgtaagtctcactacg

SEQ ID NO: 13 - DNA, 57 bp, synthetic construct
  source 1..57: mol_type = other DNA
acaggctaacgaacgatctcgagcgctttttcctacgaagctgtaagtctcactacg

SEQ ID NO: 14 - DNA, 62 bp, synthetic construct
  source 1..62: mol_type = other DNA
acaggctaacgaacgatctcgagcgcttttttttttcctacgaagctgtaagtctcactacg

SEQ ID NO: 15 - DNA, 51 bp, synthetic construct
  source 1..51: mol_type = other DNA
  misc_feature 51: note = 3 prime amino modification
acaggctaacgaacgatctcgagcgcgcgagaagttaagacctatgctcgc

SEQ ID NO: 16 - DNA, 33 bp, synthetic construct
  source 1..33: mol_type = other DNA
cctgaaccgcgagcataggtcttaacttctcgc

SEQ ID NO: 17 - DNA, 33 bp, synthetic construct
  source 1..33: mol_type = other DNA
gcgagaagttaagacctatgctcgcggttcagg

SEQ ID NO: 18 - DNA, 50 bp, synthetic construct
  source 1..50: mol_type = other DNA
  misc_feature 50: note = 3 prime RQ quencher modification
ttgggttcactgcacctggatcgagcctttacaccgactccaacagctcg

SEQ ID NO: 19 - DNA, 20 bp, synthetic construct
  source 1..20: mol_type = other DNA
  misc_feature 1: note = 5 prime azide modification
tccaggtgcagtgaacccaa

SEQ ID NO: 20 - DNA, 21 bp, synthetic construct
  source 1..21: mol_type = other DNA
ctgttggagtcggtgtaaagg

SEQ ID NO: 21 - DNA, 15 bp, synthetic construct
  source 1..15: mol_type = other DNA
tcatcgagccacaag

SEQ ID NO: 22 - DNA, 46 bp, synthetic construct
  source 1..46: mol_type = other DNA
  misc_feature 46: note = 3 prime RQ quencher modification
gtagtatatatcttgcagcgttggcgaagttaagacctatgcgcca

SEQ ID NO: 23 - DNA, 40 bp, synthetic construct
  source 1..40: mol_type = other DNA
  misc_feature 1: note = 5 prime azide modification
cgctgcaagatatatactactcgctaaactaaagattgga

SEQ ID NO: 24 - DNA, 33 bp, synthetic construct
  source 1..33: mol_type = other DNA
  misc_feature 1: note = 5 prime biotin modification
tttttttttttttttccaatctttagtttagcg

SEQ ID NO: 25 - DNA, 12 bp, synthetic construct
  source 1..12: mol_type = other DNA
  misc_feature 12: note = 3 prime RQ quencher modification
actgcacctgga

SEQ ID NO: 26 - DNA, 39 bp, synthetic construct
  source 1..39: mol_type = other DNA
ggttggtgtggttggtttttttttgtgcctgcacctgga

SEQ ID NO: 27 - DNA, 57 bp, synthetic construct
  source 1..57: mol_type = other DNA
gggttcattgcactttttttttttttttagtccgtggtagggcaggttggggtgact

SEQ ID NO: 28 - DNA, 15 bp, synthetic construct
  source 1..15: mol_type = other DNA
gtgcctgcacctgga

SEQ ID NO: 29 - DNA, 15 bp, synthetic construct
  source 1..15: mol_type = other DNA
gggttcattgcactt

SEQ ID NO: 30 - DNA, 74 bp, synthetic construct
  source 1..74: mol_type = other DNA
tggatggtagctcggtcggggtgggtgggttggcaagtcttttttttttttttttcagtgaggctgcacctgga

SEQ ID NO: 31 - DNA, 74 bp, synthetic construct
  source 1..74: mol_type = other DNA
ttgggttcattcctcactgttttttttttttttttggatggtagctcggtcggggtgggtgggttggcaagtct

SEQ ID NO: 32 - DNA, 19 bp, synthetic construct
  source 1..19: mol_type = other DNA
cagtgaggctgcacctgga

SEQ ID NO: 33 - DNA, 19 bp, synthetic construct
  source 1..19: mol_type = other DNA
ttgggttcattcctcactg

SEQ ID NO: 34 - DNA, 18 bp, synthetic construct
  source 1..18: mol_type = other DNA
  misc_feature 1: note = 5 prime biotin modification
agaatgctgagatgtaga

SEQ ID NO: 35 - DNA, 50 bp, synthetic construct; identical to SEQ ID NO: 18
  source 1..50: mol_type = other DNA

SEQ ID NO: 36 - DNA, 20 bp, synthetic construct
  source 1..20: mol_type = other DNA
tccaggtgcagtgaacccaa

SEQ ID NO: 37 - DNA, 20 bp, synthetic construct
  source 1..20: mol_type = other DNA
gcgtaatgactaactgatgg

SEQ ID NO: 38 - DNA, 13 bp, synthetic construct
  source 1..13: mol_type = other DNA
gtcgtcccgagag

SEQ ID NO: 39 - DNA, 51 bp, synthetic construct
  source 1..51: mol_type = other DNA
ctctcgggacgaccgtgtgtgttgctctgtaacagtgtccattgtcgtccc

SEQ ID NO: 40 - DNA, 51 bp, synthetic construct
  source 1..51: mol_type = other DNA
ctctcgggacgacgccagaagtttacgaggatatggtaacatagtcgtccc

SEQ ID NO: 41 - DNA, 12 bp, synthetic construct
  source 1..12: mol_type = other DNA
actaactgatgg

SEQ ID NO: 42 - DNA, 65 bp, synthetic construct
  source 1..65: mol_type = other DNA
ccatcagttagtcattacgcttacggcggctctatcctaactgatatattgtgaagtcgtgtccc

SEQ ID NO: 43 - DNA, 23 bp, synthetic construct
  source 1..23: mol_type = other DNA
cgcataggtcttaacttcgccaa

(19) United States
(12) Patent Application Publication
(10) Pub. No.: US 2023/0407378 A1
(43) Pub. Date: Dec. 21, 2023

(54) SWITCHABLE NUCLEIC ACID-SCAFFOLDED CATALYTIC SYSTEMS AND METHOD OF USE

(71) Applicant: WISCONSIN ALUMNI RESEARCH FOUNDATION, Madison, WI (US)

(72) Inventors: Jeffrey Martell, Madison, WI (US); Ashley Ogorek, Madison, WI (US); Edward Pimentel, Madison, WI (US); Caleb A. Cox, Madison, WI (US)

(21) Appl. No.: 18/052,729

(22) Filed: Nov. 4, 2022

Related U.S. Application Data
(60) Provisional application No. 63/275,722, filed on Nov. 4, 2021.

(51) Int. Cl. C12Q 1/6834; C12Q 1/6818; C12Q 1/6844; C12Q 1/6853; C12Q 1/6876; C12Q 1/70; G01N 21/64

(52) U.S. Cl. CPC C12Q 1/6834; C12Q 1/6818; C12Q 1/6846; C12Q 1/6853; C12Q 1/6876; C12Q 1/707; C12Q 1/708; G01N 21/6486

(57) ABSTRACT

The present disclosure relates to nucleic acid-scaffolded catalytic systems. In one embodiment, a nucleic acid-scaffolded catalytic system may include a nucleic acid catalyst composed of a first nucleic acid strand and, optionally, a second nucleic acid strand. The nucleic acid catalyst may further include a first reactive moiety attached to the first nucleic acid strand, and a second moiety attached to the first nucleic acid strand or the second nucleic acid strand. A catalytic activity of the first reactive moiety may be dependent on a distance between the first reactive moiety and the second moiety. The system may further include an analyte that binds to the nucleic acid catalyst to trigger a switch in the catalytic activity of the first reactive moiety by altering the distance between the first reactive moiety and the second moiety.

### CROSS-REFERENCE TO RELATED APPLICATIONS

The present application claims priority to U.S. Provisional Patent Application No. 63/275,722 filed Nov. 4, 2021, the entire contents of which are hereby incorporated by reference.

### STATEMENT REGARDING FEDERALLY SPONSORED RESEARCH

This invention was made with government support under W911NF-21-1-0073 awarded by the ARMY/ARO. The government has certain rights in the invention.

### REFERENCE TO AN ELECTRONIC SEQUENCE LISTING

The contents of the electronic sequence listing (960296.04363.xml; Size: 44,097 bytes; and Date of Creation: Nov. 4, 2022) is herein incorporated by reference in its entirety.

### FIELD OF INVENTION

The present disclosure generally relates to nucleic acid-scaffolded catalytic systems. The present disclosure further relates to methods of using nucleic acid-scaffolded catalytic systems for applications such as controlling reaction rates, sensing, biomarker detection, protein-protein interaction detection, and proximity labeling.

### BACKGROUND

Catalysts play an important role in the effective and efficient transformation of reactants to intermediates and products. Catalysts are often used to convert readily available (often lower value) starting materials to products that possess more desirable characteristics (e.g., functionality). The creation of catalysts that mimic enzymes through pre-organization of reactive groups is a long-standing goal. In natural enzymes, protein scaffolds hold multiple functional groups in proximity, providing massive rate accelerations that are not observed when the same functional groups are unscaffolded. Catalyst research often focuses on the identification of new catalysts or the optimization of performance to achieve higher conversions and enhanced selectivity. Some researchers have turned their attention to the development of catalysts whose activity is controlled by external stimuli. Such switchable catalysts are of interest in a variety of applications. Catalysts that combine biological functions/cues with synthetic catalysts have the potential to enable or improve a variety of reactions and processes.

While matching the sophistication of enzymes may be an elusive target for synthetic systems, supramolecular enzyme mimics offer a broader palette of functional groups since they are not limited to natural amino acids and cofactors. Impressive catalytic properties have been demonstrated in a variety of enzyme-mimicking supramolecular scaffolds.

An exciting area of recent progress in supramolecular chemistry is the pre-organization of synergistic catalytic reactions. Synergistic catalysis combines two co-catalysts to carry out a reaction that would not be possible using either co-catalyst alone. Examples include a variety of synergistic combinations of transition metal catalysts, photocatalysts, organocatalysts, radical catalysts, and Lewis acid catalysts. In scaffolded synergistic catalysis, the proximity afforded by precise positioning of the co-catalysts accelerates the reaction compared to the unscaffolded reactions, in which the large mean distance between the co-catalysts limits synergistic interactions, often necessitating high catalyst loadings. However, it remains a challenge to fine-tune the spacing of each co-catalyst. Furthermore, previously demonstrated scaffolds for synergistic catalytic reactions are difficult to engineer for stimuli-triggered conformational changes to alter the rate of the reaction. Allostery is an attractive property of enzyme catalysis and is of fundamental interest for enzyme-mimicking systems and for the development of sensors and responsive materials.

Thus, there is a need for designed catalytic systems that provide precise positioning of reactive functional groups and exhibit conformational and catalytic activity switching in response to external stimuli.

### SUMMARY

Disclosed herein is a nucleic acid-scaffolded catalytic system. In one aspect, the nucleic acid-scaffolded catalytic system includes a nucleic acid catalyst including a first nucleic acid strand and, optionally, a second nucleic acid strand, a first reactive moiety attached to the first nucleic acid strand, and a second moiety attached to the first nucleic acid strand or the second nucleic acid strand, wherein a catalytic activity of the first reactive moiety is dependent on a distance between the first reactive moiety and the second moiety.

A trigger may bind to the nucleic acid structure and trigger an increase in catalytic activity of the first reactive moiety. A ‘trigger’, which may also be referred to as an ‘analyte,’ may bind to either or both of the first nucleic acid strand and the second nucleic acid strand. A trigger may increase or decrease the catalytic activity of the first reactive moiety by altering the distance between the first reactive moiety and the second moiety. The trigger may include an ion, a small molecule, a protein, and a nucleic acid. In certain examples, the trigger may be a single stranded DNA or RNA oligomer that anneals with the first nucleic acid strand or to both of the first nucleic acid strand and the second nucleic acid strand.

In certain examples, the first reactive moiety may be a catalyst. In other examples, the first reactive moiety and the second moiety may be abiotic groups. In certain cases, the first reactive moiety may be attached to the 5′ end of the first nucleic acid strand and the second moiety may be attached to the 3′ end of the first nucleic acid strand. In other cases, the reverse may be true where the first reactive moiety may be attached to the 3′ end of the first nucleic acid strand and the second moiety may be attached to the 5′ end of the first nucleic acid strand.

In certain examples the nucleic acid scaffold the first nucleic acid strand may be folded into a stem-loop structure. The first reactive moiety may be attached to a first end of the first nucleic acid strand and the second reactive moiety may be attached to a second end of the first nucleic acid strand such that the first reactive moiety and the second moiety are in proximity in the stem-loop structure. When a trigger, which may be a template nucleic acid strand, anneals with the first nucleic acid strand, the stem-loop structure may open to move the second moiety farther away from the first reactive moiety.

In certain examples, the nucleic acid-scaffolded catalytic system may include the first reactive moiety attached to the first nuclei acid strand, the second moiety attached to the second nucleic acid strand. When a trigger (that may be a template nucleic acid strand) anneals with the first nucleic acid strand and the second nucleic acid strand, the first reactive moiety may be brought into proximity to the second moiety. In certain cases, a separation of five or more nucleotides between the first reactive moiety and the second moiety diminishes the catalytic activity of the first reactive moiety.

In another example, the first reactive moiety may catalyze the production of a colored or fluorescent product. In certain cases, the first reactive moiety may be a photocatalyst and the second moiety may be a quencher of the photocatalyst. One example may include the photocatalyst Eosin Y and the quencher may be black hole quencher 1. In another example, the photocatalyst may be a ruthenium catalyst and the quencher may be black hole quencher 2. In other examples, the first reactive moiety may be a photocatalyst and the second moiety may be a Forster resonance energy transfer (FRET) donor. In certain cases, the photocatalyst may be a ruthenium complex. In a certain case, the first reactive moiety may be a ruthenium photocatalyst and the second moiety may be a cobalt catalyst, and the ruthenium photocatalyst and the cobalt catalyst synergistically convert tetrahydroisoquinoline to isoquinoline when in proximity to each other. In another example, the first reactive moiety may be an iridium photocatalyst and the second moiety may be a nickel complex, and the iridium photocatalyst and the nickel complex synergistically catalyze a bond-forming reaction when in proximity to each other.

In certain cases, the first reactive moiety and the second reactive moiety may synergistically catalyze a reaction when in proximity to each other.

In other cases, the first reactive moiety may catalyze the conversion of a prodrug into an efficacious drug.

In certain examples, the first reactive moiety may be a palladium complex and the second moiety may be an activating ligand of the palladium complex. The palladium complex may catalyze removal of a propargyl group from a substrate when activated by the activating ligand.

The nucleic acid-scaffolded catalytic system may include a first reactive moiety that may catalyze the formation of a polymer. In some examples, the first reactive moiety may catalyze the polymerization of diaminobenzidine (DAB). The first reactive moiety may catalyze the polymerization of 3,3′-diaminobenzidine. In other examples, the first reactive moiety may catalyze the formation of a cross-linked polymer hydrogel. In certain cases, the first reactive moiety may catalyze poly(ethylene glycol) diacrylate cross-linking to form a hydrogel. In another example, the first reactive moiety may catalyze the synthesis of a controlled polymer, such as a polymer synthesized through Photoinduced electron/energy transfer reversible addition—fragmentation chain transfer polymerization.

An example of the disclosed technology may include the first reactive moiety being 2,2,6,6-tetramethylpiperidiny-1-yl)oxyl (TEMPO) organic radical and the second moiety may be a copper co-catalyst. In this example, the TEMPO organic radical and the copper co-catalyst may catalyze the oxidation of an alcohol to an aldehyde. The TEMPO radical may be attached to the first nucleic acid strand and the copper co-catalyst is attached to the second nucleic acid strand. The analyte may be a template nucleic acid strand that anneals with the first nucleic acid strand and the second nucleic acid strand to bring the TEMPO radical and the copper co-catalyst into proximity. In a further aspect of this example, the catalytic system may further include an invader nucleic acid strand that displaces the template nucleic acid strand and separates the TEMPO radical from the copper co-catalyst. In a certain case, a trigger strand that anneals with one of the first nucleic acid strand and the second nucleic acid strand to separate the first reactive moiety from the second moiety may switching off catalysis of the reaction.

In another aspect, the nucleic acid-scaffolded catalytic system may further include a first split invader construct that includes a first protein binding moiety linked with a nucleic acid strand having a first complementary domain and a first complementary catalyst domain opposite the first protein binding domain and a second split invader construct that includes a second protein binding moiety linked with a nucleic acid strand having a second complementary domain and a second complementary catalyst domain opposite the second protein binding moiety. The first protein binding moiety and the second protein binding moiety may bind to sites on one or more proteins. The first complementary domain and the second complementary domain are complementary to one another and when the first complementary domain and second complementary domain are annealed with each other, the first split invader construct and second split invader construct form a full invader duplex. The full invader duplex may trigger the nucleic acid-scaffolded catalytic system by binding the first complementary catalyst domain and the second complementary catalyst domain of the full invader duplex to either or both of the first nucleic acid strand and the second nucleic acid strand to trigger an increase or a decrease in the catalytic activity of the first reactive moiety by altering the distance between the first reactive moiety and the second moiety.

The first protein binding moiety and the second protein binding moiety may be an aptamer or an antibody.

One aspect of the disclosed technology includes a method for controlling a rate of a reaction with a nucleic acid-scaffolded catalyst, including: triggering a conformational shift in the nucleic acid-scaffolded system according as described earlier by addition of a trigger that binds to the nucleic acid scaffold, the conformational shift in the nucleic acid scaffold changing the rate of the reaction by changing the distance between the first reactive moiety and the second moiety.

One example of this method may involve the first reactive moiety being a photocatalyst and the method further comprises irradiating the photocatalyst.

One aspect of the disclosed technology includes a method for detecting an analyte, involving exposing the nucleic acid scaffold system described above to a sample. If the sample contains the analyte, then the analyte may bind to the nucleic acid scaffold and trigger a conformational shift in the nucleic acid scaffold that changes the distance between the first reactive moiety and the second moiety and the catalytic activity of the first reactive moiety, and detecting the presence of the analyte based on the change in catalytic activity of the first reactive moiety.

Another aspect of the disclosed technology includes a method for detecting a protein-protein interaction. The method includes exposing the nucleic acid-scaffolded system described previously to a sample with the one or more proteins. Trimolecular annealing between the first complementary catalyst domain, the second complementary catalyst domain, and either or both of the first nucleic acid strand and the second nucleic acid strand may trigger a conformational shift that changes the distance between the first reactive moiety and the second moiety and detecting the trimolecular annealing based on the change in catalytic activity of the first reactive moiety. One example of this methods of using the disclosed technology includes detecting photocatalytic labeling. This method may also involve detection of fluorescence.

An example of the immediately preceding method where there are two proteins includes the binding of the first protein to the second protein may lead to an increase or decrease in the catalytic activity of the first reactive moiety. The first protein and the second protein may be extracellular proteins. The extracellular proteins may be two different cell surface antigens, where the first nucleic acid strand and the trigger strand are tethered to respective antibodies for the two different cell surface antigens. The interaction between the first protein and the second protein may be an intercellular protein-protein interaction, a protein-ligand interaction on a cell surface, neuronal proteins at a synaptic cleft, are biomarkers on a same cell (e.g., the biomarkers are biomarkers for circulating tumor cells). In another example, the first reactive moiety may catalyze formation of a magnetic coating on the cell to identify the cell as a circulating tumor cell

In a certain example, the first nucleic acid strand may be conjugated to an antibody and bound to a cell via an interaction between the antibody and an antigen. The trigger nucleic acid strand may be conjugated to an antibody and bound to a cell via an interaction between the antibody and an antigen. One example of this method involves the one or more proteins being located on one or more cell surfaces.

### BRIEF DESCRIPTION OF THE DRAWINGS

FIG. 1 is a schematic representation of an exemplary switchable nucleic acid-scaffolded catalytic system, in accordance with the present disclosure.

FIG. 2 is a schematic representation of another exemplary switchable nucleic acid-scaffolded catalytic system, in accordance with the present disclosure.

FIG. 3 is a schematic representation of another exemplary switchable nucleic acid-scaffolded catalytic system, in accordance with the present disclosure.

FIG. 4 is a schematic representation of another exemplary switchable nucleic acid-scaffolded catalytic system, in accordance with the present disclosure.

FIGS. 5-7 are schematic representations of exemplary nucleic acid-scaffolded catalytic systems, in accordance with the present disclosure.

FIG. 8 shows switching of catalytic activity in the presence of a template nucleic acid strand in a nucleic acid-scaffolded photocatalytic system, in accordance with the present disclosure.

FIG. 9 is a schematic representation of another exemplary nucleic acid-scaffolded photocatalytic system, in accordance with the present disclosure.

FIG. 10 is a schematic representation of another exemplary nucleic acid-scaffolded photocatalytic system, in accordance with the present disclosure.

FIG. 11 shows triggering of 3,3′-diaminobenzidine (DAB) polymerization in the nucleic acid-scaffolded photocatalytic system of FIG. 10, in accordance with the present disclosure.

FIG. 12 shows triggering of polyacrylate cross-linking to form a hydrogel in the nucleic acid-scaffolded photocatalytic system of FIG. 10, in accordance with the present disclosure.

FIG. 13 shows photocatalyzed aryl azide reduction with the nucleic acid-scaffolded photocatalytic system of FIG. 10, in accordance with the present disclosure.

FIG. 14 is a schematic representation of another exemplary nucleic acid-scaffolded photocatalytic system, in accordance with an embodiment the present disclosure.

FIG. 15 is a schematic representation of another exemplary nucleic acid-scaffolded photocatalytic system, in accordance with the present disclosure.

FIG. 16 shows a schematic representation of another switchable nucleic acid-scaffolded catalytic system, in accordance with the present disclosure.

FIG. 17 is a schematic representation of another switchable nucleic acid-scaffolded synergistic catalytic system, in accordance with the present disclosure.

FIG. 18 shows a reaction catalyzed by a nucleic acid-scaffolded synergistic catalytic system having TEMPO and copper complex co-catalysts, in accordance with the present disclosure. The nucleic acid scaffold is a DNA duplex formed with SEQ ID NO: 5 and SEQ ID NO: 6.

FIG. 19 shows measurements of 2-napthalenemethanol oxidation for nucleic acid-scaffolded copper complex-TEMPO co-catalysts (DNA duplex scaffold formed with SEQ ID NO: 5 and SEQ ID NO: 6), a nucleic acid scaffold with the two catalysts tethered to opposite 5′ ends (DNA duplex scaffold formed with SEQ ID NO: 6 and SEQ ID NO: 7), and the unscaffolded catalysts, in accordance with the present disclosure.

FIG. 20 shows catalyst turnover number data demonstrating an increase in turnover number for 2-napthalenemethanol oxidation when the copper complex-TEMPO co-catalysts are in proximity to each other on the nucleic acid scaffold, in accordance with the present disclosure. Catalyst turnover number is measured at 23 hours for negative control catalysts. Data are presented as the mean±standard deviation of three independent experiments.

FIG. 21 shows a comparison of nucleic acid-scaffolded and unscaffolded oxidation of various benzylic alcohols after 4.75 hours of reaction. The fold increase afforded by the nucleic acid scaffold relative to the unscaffolded reaction is shown for each substrate. Reaction conditions were 10 mM substrate, 0.5 mol % nucleic acid-scaffolded copper complex-TEMPO co-catalysts (scaffolded) or 0.5 mol % 4,4′-dicarboxylic acid-2,2′-bypyridine (dcbpy) and cTEMPO (unscaffolded), 0.5 mol % copper bromide (CuBr) in 1:1 acetonitrile/100 mM sodium borate buffer, pH 9.5 with 100 mM sodium chloride (NaCl).

FIG. 22 shows the effect of catalyst loading on the turnover number of scaffolded and unscaffolded oxidation of 2-napthalenemethanol. Catalyst loadings indicated the mol % loading for bipyridine (bpy) ligand, TEMPO, and copper(I). The fold increase in catalyst turnover number to nucleic acid scaffolding is indicated for each catalyst loading. Data are presented as the mean±standard deviation of three independent experiments.

FIG. 23 is a calibration curve to determine the effective concentration of nitroxyl radical (TEMPO) catalyst when scaffolded on DNA. DNA-linked dcbpy (2 nanomole (nmol)) was incubated with CuBr (2 nanomol) and varying concentrations of TEMPO as co-catalyst. The resulting linear regression was used to extrapolate the effective concentration of TEMPO on DNA from the observed rate of catalysis using scaffolded catalysts. Reaction conditions were 10 mM 2-napthalenemethanol, 0.5 mol % DNA-linked dcbpy (with complementary unmodified strand), mol % CuBr, and varied loadings of TEMPO in 1:1 acetonitrile/100 mM sodium borate buffer, pH 9.5 with 100 mM NaCl.

FIG. 24 shows a calibration curve to determine the effective concentration of the 4,4′-dicarboxy-2,2′-bipyridine-copper catalyst when scaffolded on DNA. DNA-linked cTEMPO (2 nmol) was incubated with varying concentrations of CuBr and dcbpy. The resulting linear regression was used to extrapolate the effective concentration of the dcbpy-Cu catalyst on DNA from the observed rate of catalysis using scaffolded catalysts. Reactions conditions were 10 mM 2-napthalenemethanol, 0.5 mol % DNA-cTEMPO (with complementary unmodified strand), and varied, equimolar loadings of dcbpy and CuBr in 1:1 acetonitrile/100 mM sodium borate buffer, pH 9.5 with 100 mM NaCl.

FIG. 25 shows tuning of inter-catalyst distance using template strands with single-stranded polythymidine spacers. Catalyst-bearing single-stranded DNA oligomers were annealed to a template single stranded DNA, bringing the catalysts into proximity to provide three-stranded DNA-scaffolded copper-TEMPO catalysts. Catalyst turnover number for 2-napthalenemethanol oxidation by the three-stranded DNA-scaffolded copper-TEMPO catalysts with polythymidine spacers of varying lengths is shown. Data are plotted as the mean±standard deviation of three independent experiments.

FIG. 26 is a schematic representation of a switchable nucleic acid-scaffolded synergistic catalytic system, in accordance with the present disclosure. The distance between the two catalysts is increased upon addition of a single-stranded DNA trigger strand (switch state “off”). The original scaffold conformation is restored upon addition of an anti-trigger strand (switch state “on”).

FIG. 27 shows catalyst turnover number for oxidation of 2-napthalenemethanol quantified through multiple cycles of conformational changes of the switchable nucleic acid-scaffolded catalyst of FIG. 26. Conformational changes are triggered by successive addition of the trigger and anti-trigger strands. Data are plotted as the mean±standard deviation of three independent experiments.

FIG. 28 shows a schematic representation of a system for detecting an extracellular protein-protein interaction (PPI) using a nucleic acid-scaffolded catalyst, in accordance with the present disclosure.

FIG. 29 is a schematic representation of catalysis activation of a nucleic acid-scaffolded catalyst in the presence of two cell surface antigens, in accordance with the present disclosure.

FIG. 30 shows nucleic acid-antibody conjugates, in accordance with the present disclosure.

FIG. 31 shows nucleic-antibody conjugates bound to proximal HA and myc epitopes, in accordance with the present disclosure.

FIG. 32 shows fluorescence results demonstrating proximity-based catalytic activation in the system of FIG. 31, in accordance with the present disclosure.

FIG. 33 shows classes of protein-protein interactions (PPIs) that may be studied using nucleic acid-scaffolded catalysts of the present disclosure.

FIG. 34 shows an application of nucleic acid-scaffolded catalysts for investigating PPIs between neurons at a synaptic cleft, in accordance with the present disclosure.

FIG. 35 shows an application of nucleic acid-scaffolded catalysts for proximity labeling at intercellular PPIs, in accordance with the present disclosure.

FIG. 36 is a schematic representation of identifying circulating tumor cells (CTCs) using a nucleic acid-scaffolded catalyst, in accordance with the present disclosure.

FIG. 37 is a schematic showing switchable nanocatalysts for spatially resolved proteomic mapping of immune cell-cancer cell contacts. (a) Existing methods employ proximity labeling catalysts that are always active, leading to biotin tagging of the entire cancer cell surface. (b) The present switchable proximity labeling catalysts can be used that only turn on at immune cell-cancer cell contact sites, enabling proximity biotin tagging exclusively at these contact sites. After subsequent cell lysis, harvesting of biotin-tagged peptides, and high-resolution mass spectrometry, highly specific proteomic lists will be obtained.

FIG. 38 is a schematic of the mechanism of activation for switchable DNA nanocatalysts. (a) The DNA nanocatalyst changes its conformation and becomes catalytically active upon binding to an immune cell protein “trigger,” leading to generation of a reactive biotin probe. (b) The DNA nanocatalyst activates only at locations where the immune cell protein “trigger” is present, enabling spatially-restricted biotin tagging exclusively at immune cell-cancer cell contact sites.

FIG. 39 shows an overview of switchable DNA PL catalysts. Panel A shows conventional proximity labeling (PL) catalysts are constitutively active; they are attached to a POI, and PL occurs at any subcellular location where the POI is present. Panel B shows the inventors approach uses a switchable DNA catalyst that can be attached to a POI, but only becomes active for PL when a molecular trigger is present. Panel C shows the DNA catalyst can be activated at sites of protein-protein interactions, specifically c-Met homodimers.

FIG. 40 shows in vitro development of DNA switchable PL catalyst. Panel A shows the design of a switchable DNA catalyst that is activated by a ssDNA trigger. In the absence of trigger DNA, the photocatalyst and quencher are held in proximity via DNA hybridization. In the presence of trigger, the quencher is distanced from the photocatalyst, turning on the activity of the photocatalyst for generating phenoxy radicals. Panel B show the structure of photocatalyst Ru(bpy)2(phen) that is attached to DNA. Panel C shows a scheme of triggered biotinylation of BSA. Panel D shows Western Blot of BSA following BP labeling in the presence of various quenched and triggered catalysts. Upper panel: Ponceau stain. Lower panel: streptavidin-HRP stain. Panel E shows Ru luminescence of quenched and triggered DNA photocatalyst.

FIG. 41 shows triggered proximity labeling on streptavidin-coated beads. Panel A shows quenched DNA catalyst is localized to streptavidin-coated magnetic beads through hybridization to a biotinylated anchor strand of ssDNA. The trigger strand of ssDNA activates the photocatalyst, enabling biotinylation of streptavidin followed by staining with fluorescent sAv-PE. Panel B shows a histogram of flow cytometry data of beads after exposure to BP labeling conditions and sAv-PE staining. 20,000 events per sample were recorded, and fluorescence of the sAv-PE channel is shown. i) quenched DNA catalyst, ii) triggered DNA catalyst, iii) DNA structure lacking Ru photocatalyst, iv) triggered DNA catalyst, no light during BP labeling. Fluorescence detection: excitation 561 nm, emission 577 nm. Panel C shows experiments i-iv from panel B were performed in triplicate. Within each replicate of the experiment, the mean fluorescence from the 20,000 detected events was determined. Next, the average of the mean fluorescence values was determined across the three triplicates, for conditions i through iv. Data are presented as the average±standard derivation of the mean bead intensities for each replicate.

FIG. 42 shows validation of split invader system using thrombin-coated beads. Panel A shows two split invaders (A and B) are appended to the DNA aptamers HD1 and HD22, which bind to distinct sites on thrombin. Binding of the aptamers to thrombin brings the split invader constructs into proximity, leading to formation of a stable duplex (C—C*). This configuration allows for annealing of the Ru-quencher duplex onto the reconstituted invader, followed by toehold displacement of the quencher stand, resulting in an activated DNA catalyst localized to thrombin. The active DNA catalyst biotinylates thrombin; biotin labeling is detected by staining with sAv-PE. Panel B shows a histogram of flow cytometry data of beads after BP labeling and sAv-PE staining. 100,000 events per sample were recorded, and fluorescence of the sAv-PE channel is shown. i) HD1-CA+HD22-C*B, ii) HD1-CA+C*B, iii) CA+HD22-C*B, iv) unlabeled thrombin-coated beads. Fluorescence detection: excitation 561 nm, emission 585 nm. Panel C shows each experiment from panel B was performed in duplicate. Within each replicate of the experiment, the mean fluorescence from the 100,000 detected events was determined. Next, the average of the mean fluorescence values was determined across the two duplicates, for conditions i through iv. Data are presented as the average±standard derivation of the mean bead intensities for each replicate.

FIG. 43 shows activating PL at c-Met homodimers on living mammalian cells. Panel A shows an intact invader strand is reconstituted due to the proximity of two split triggers, which bind to c-Met via DNA aptamer targeting. The activated DNA catalyst is recruited to the site of c-Met homodimers via toehold strand displacement of the quencher strand. The activated DNA catalyst biotinylates cell surface proteins followed by staining with sAv-PE. Panel B shows epi-fluorescence microscopy images of c-Met and BFP-NLS transfected cells after exposure to complete or incomplete split invaders followed by BP and sAv-PE labeling. Left column: Apt-CA+Apt-C*B. Middle column: Apt-CA+C*B. Right column: CA+Apt-C*B. Red: sAv-PE. Blue: BFP. Scale bars represent 50 μm.

FIG. 44 shows an illustration of switchable DNA scaffolded photocatalysts. DNA hybridization brings a photocatalyst dye in proximity of a quencher dye, causing FRET to occur and switching the photocatalyst to an “OFF” state. Upon binding of the target, the proximity of the quencher dye is removed by a change in DNA conformation, switching the photocatalyst to an “ON” state. The switchable catalyst can be repurposed for a different target by changing the DNA sequence.

FIG. 45 shows DNA fluorescence and photocatalysis is switched-on by addition of target. Illustration of the switching mechanism is shown (top left) where binding of “target” causes a conformational change in DNA to remove the proximity of a quencher dye and fluorescein. Targets, switchable DNA catalysts, fluorescence response, and photocatalysis comparison is shown (bottom panel). Targets and DNA aptamer sensors for glucose (1 M) (SEQ ID NO: 38 and SEQ ID NO: 39_, hydrocortisone (50 μM) (SEQ ID NO: 38 and SEQ ID NO: 40), Zn²⁺ (2 mM) (SEQ ID NO: 41 and SEQ ID NO: 42), and DNA (200 nM) (SEQ ID NO: 37) targets are shown (bottom left). Secondary structure of each DNA catalyst was predicted using Mfold. Fluorescence turn-on and photocatalysis are shown (bottom right). Photocatalysis was demonstrated using a photo-polymerization (scheme, top right); red dashed lines were added to highlight the level of the liquid or hydrogel in angled vials.

FIG. 46 shows the specificity of switchable DNA catalyst for hydrocortisone. Fluorescence and photocatalysis is switched on in the presence of hydrocortisone (50 μM) but not glucose (1 M), Zn²⁺ (2 mM), non-complementary DNA (200 nM), or a structurally similar steroid, deoxycholic acid (50 μM). Hydrocortisone target is SEQ ID NO: 38 and SEQ ID NO: 40 and non-complementary DNA is SEQ ID NO: 37.

FIG. 47 shows an illustration of photocatalyst labeled yeast cells and subsequent hydrogel formation. Illustration of cell surface labeling (left) through a biotin-streptavidin-biotin sandwich. Photocatalyst states are shown as triggered, quenched, missing Biotin-NHS labeling, and unlabeled. Fluorescence from flow cytometry (middle) shows the signal from Cy3 fluorophore labeling of yeast cells in the states shown from the illustration. Photocatalysis (right) from fluorescein labeled photocatalysts on yeast cells is shown. Hydrogel formation is only seen for samples in the “ON” state (triggered). Red dashed lines were added to highlight the level of liquid or hydrogel in angled vials.

FIG. 48 shows switchable PET-RAFT polymerization through a reversible, dual-stimuli system. Panel A shows a synthetic scheme of the PET-RAFT polymerization. Panel B shows pseudo-first order kinetics are shown as the average of triplicate with ±one standard deviation. Polymerization was controlled by an AND-gate type system where both the light and the photocatalyst needed to be in an “ON” state. Photocatalyst state was reversibly controlled by the addition of chemical stimuli (DNA trigger) to modulate the proximity of a quencher dye by changes in DNA conformational state. Significant monomer conversion was only observed when light and photocatalyst were both in the ON state: 0 hr-0.5 hr, 2 hr-2.5 hr, 4 hr-4.5 hr.

### DETAILED DESCRIPTION

The present disclosure provides switchable nucleic acid-scaffolded catalytic systems and methods of use. These catalytic systems described herein are switchable, i.e., their activity is controlled by an external stimuli. The system may include a nucleic acid catalyst including a first nucleic acid strand, an optional second nucleic acid strand, a first reactive moiety attached to the first nucleic acid strand, and a second moiety attached to either the other end of the first nucleic acid strand or the second nucleic acid strand. In one aspect, the first reactive moiety is attached to one end of the first nucleic acid strand while the second reactive moiety is attached to the other end of the first nucleic acid strand, such that, when the first nucleic acid strand changes confirmation after a stimuli, can be activated or inactivated. The catalytic activity of the first reactive moiety may be dependent on a distance between the first reactive moiety and the second moiety. The system may further include a stimuli, for example, an analyte that binds to either or both of the first nucleic acid strand and the second nucleic acid strand to trigger conformational changes and thus an increase or a decrease in the catalytic activity of the first reactive moiety by altering the distance between the first reactive moiety and the second moiety. Suitable depictions of the nucleic-acid scaffolded catalytic system are illustrated in, for example, in the Figures and Examples described herein.

The switchable nucleic acid-scaffolded catalytic systems of the present invention can be used for a number of different applications and methods as described more herein. For example, the switchable nucleic acid-scaffolded catalytic systems may be used for fluorescent or colorimetric signal amplification for diagnostics as demonstrated in the examples. Further, the switchable nucleic acid-scaffolded catalytic systems can be used for spatially-resolved proteomic mapping. Methods of using the switchable nucleic acid-scaffolded catalytic systems further include the ability to polymerize diaminobenzidine to create contrast for electron microscopy. Further, the switchable nucleic acid-scaffolded catalytic systems may be used for synthesizing polymer coatings on cell surfaces for identification and isolation of rare cell types described more herein.

As used herein, the term “nucleic acid” refers to a polymer of nucleic acid bases such as oligonucleotides including, but not limited to, single-stranded DNA, double-stranded DNA, RNA, aptamers, and peptide nucleic acids (PNAs). The nucleic acid bases may include natural nucleic acid bases (e.g., adenine, guanine, cytosine, thymine), unnatural nucleic acid bases, or combinations thereof. DNA aptamers are about 15-45 nucleotide DNA oligomer that has been selected for its binding affinity toward a molecule of interest, such as, but not limited to, proteins or small molecules.

As used herein, the term “abiotic group” is a functional group that is not derived from a biological or living organism.

As used herein, a “switchable nucleic acid-scaffolded catalytic system” is used interchangeably with “nucleic acid-scaffolded catalytic system” and “nanocatalyst system” and is a nucleic acid-based structure that bears at least one reactive moiety and exhibits a change in conformation in response to an external stimulus. The catalyst is thus able to trigger a chemical reaction and the DNA nanocatalyst change in conformation allows for the triggering and control of the catalytic activity. The conformational change results in a change in the catalytic activity (from catalytically inactive to catalytically active, or from catalytically active to catalytically inactive) by changing a distance between the reactive moiety and another moiety that influences the catalytic activity of the reactive moiety. The reactive moiety is precisely positioned with respect to the influencing moiety in either or both of the catalytically inactive and active states of the system via the structural programmability of the nucleic acid-based structure.

A “nucleic acid-scaffolded catalyst”, “scaffolded catalyst”, or “catalyst” as used herein includes a nucleic acid scaffold formed of one or more nucleic acid strands and bearing at least a first reactive moiety that catalyzes a reaction when activated through a conformational change in the nucleic acid scaffold. Suitable reactive moieties include catalyst known in the art and as described in the Examples. In some embodiments, the catalyst includes a first reactive moiety and a second reactive moiety, wherein the association (or de-association) of the first and second moiety results in the catalytic activity. In some embodiments, the distance between the first catalytic moiety and second catalytic moiety can control the rate of the reaction of the catalytic reaction.

A “nucleic acid scaffold” as used herein includes one or more nucleic acid strands having a defined structure to which one or more reactive moieties having catalytic activity are attached. The nucleic acid scaffold is designed to undergo a conformational change in response to external stimuli, such as analyte binding. In one embodiment, the individual molecular beacon/hairpin and anchor components are between 30 and 55 nucleotides long, and when they hybridize together will emulate a single stand of DNA that is about 80-90 nucleotides long (accounting for double stranded portions that are annealed). In some examples, the trigger strands are equivalent to 30-100 nucleotides long. In some studies with simpler templates, only one strand of DNA (e.g. Biotin/SAV system), the molecular beacon/hairpin can be about 43 nucleotides, and the triggers are 25-43 nucleotides long.

As used herein, an “analyte” or “trigger” may be a chemical substance that binds the nucleic acid-scaffolded catalyst to induce a conformational change that results in a change in catalytic activity of the catalyst. The choice of the term analyte or trigger may depend upon context or be used interchangeably. For example, the term trigger may be used when the chemical substance is used to control the activity of the system and the term analyte may be used when it is desirable to identify the presence or amount of the chemical substance. Examples of analytes or triggers include, but are not limited to, ions, small molecules, proteins, and nucleic acids (e.g., single stranded DNA, RNA, aptamers, peptide nucleic acids, etc.). In some arrangements, the analyte or trigger is a template nucleic acid strand or a nucleic acid strand that anneals to the nucleic acid-scaffolded catalyst. Other examples of analytes or triggers for switchable scaffolded catalysts include metal ions, glucose (including the D-glucose enantiomer), hydrocortisone, and complementary DNA.

In some embodiments, the conformation in the nucleic acid-scaffolded catalyst may be induced by an analyte or a trigger, as explained above, or it may be reversed by an “anti-trigger”. In some embodiments the anti-trigger turns off the catalytic activity while in other embodiments the anti-trigger turns on the catalytic activity.

“Synergistic catalysis” as used herein refers to the combination of two catalytic components (or co-catalysts) to drive reactions that do not occur with either component alone.

In some embodiments, the switchable DNA catalysts of the present disclosure can use, but are not limited to, increasing the distance between a photocatalyst and a spectral quencher, bringing a photocatalyst close to a FRET donor, bringing two synergistic co-catalysts (such as a copper co-catalyst and a nitroxyl radical co-catalyst for synergistic alcohol oxidation) close together, and altering the coordination environment of a transition metal, for catalytic switching.

In some embodiments, the switchable DNA catalysts of the present disclosure can be used in a number of different biological or chemical processes. For example, but not limited thereto, the switchable catalysts may be used for unmasking a prodrug for therapeutics, for transcriptional control of immune chell therapies, for diagnostics, for proximity labeling for prteomic mapping and for switchable polymerization (e.g., for self-healing materials).

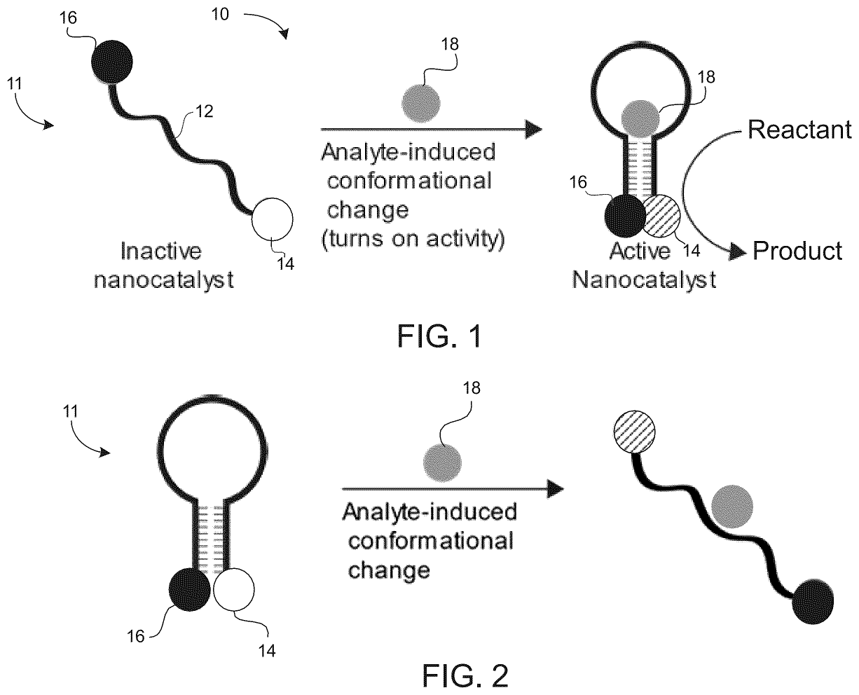

Referring now to the drawings, and with specific reference to FIG. 1, a switchable nucleic acid-scaffolded catalytic system 10 is shown. In this embodiment, the catalytic system 10 includes a catalyst 11 that includes a first nucleic acid strand 12 attached to a first reactive moiety 14 and a second moiety 16. In some aspects, the first reactive moiety 14 is on the opposite endo of the nucleic acid strand than the second moiety 16. The catalytic activity of the first reactive moiety 14 is dependent on its proximity to the second moiety 16. In some embodiments, the first reactive moiety 14 may be catalytically activated (or inactivated) when it is within about 2 to about 10 nanometers (nm) of the second moiety 16, and may be catalytically inactivated (or activated) when it is at a distance greater than about 2 to about 10 nm from the second moiety 16.

Upon addition of an analyte 18 or other stimuli, a conformational change in the nucleic acid strand 12 is triggered which changes the distance between the first reactive moiety 14 and the second moiety 16, thereby triggering a change (increase or decrease) in catalytic activity of the catalyst 11. For instance, as shown in FIG. 1, binding of the analyte 18 to the first nucleic acid strand 12 triggers a change from a random conformation to a stem-loop or hairpin conformation which places the first reactive moiety 14 in proximity to the second moiety 16. Depending on how the second moiety 16 influences the catalytic activity of the first reactive moiety 14, the conformational switch may either: 1) increase the catalytic activity of the first reactive moiety 14 leading to an increase in product conversion, or 2) decrease the catalytic activity of the first reactive moiety 14, leading to a decrease in product conversion. Thus, binding of the analyte 18 may trigger switching of the catalyst 11 from an inactive catalytic state to an active catalytic state, or switching from an active catalytic state to an inactive catalytic state.

In another embodiment shown in FIG. 2, binding of the analyte 18 may induce conformational shifting of the catalyst 11 from an initial stem-loop or hairpin conformation with the first reactive moiety 14 and the second moiety 16 in proximity, to a random or extended conformation with the first reactive moiety 14 and the second moiety 16 separated from each other. As above, binding of the analyte 18 may trigger conformational shifting from an inactive catalytic state to an active catalytic state, or from an active catalytic state to an inactive catalytic state. In either of the embodiments of FIGS. 1 and 2, the first reactive moiety 14 and the second moiety 16 may be tethered to the termini or ends of the nucleic acid strand 12. For instance, the first reactive moiety 14 may be attached to the 5′ end of the nucleic acid strand 12 and the second moiety 16 may be attached to the 3′ end of the nucleic acid strand 12. Alternatively, the first reactive moiety 14 may the attached to the 3′ end of the nucleic acid strand 12 and the second moiety 16 may be attached to the 5′ end of the nucleic acid strand 12.

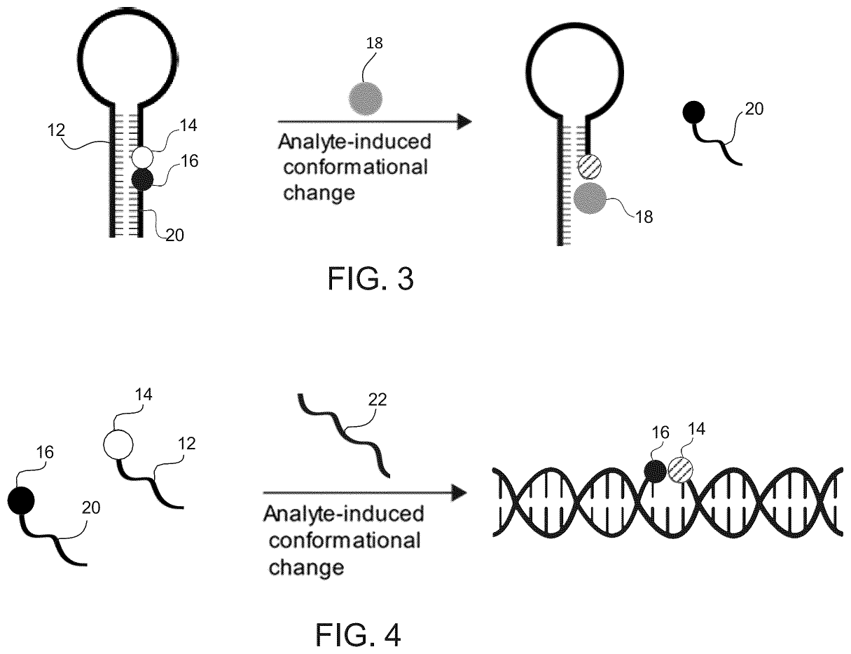

In yet another embodiment shown in FIG. 3, the catalytic system 10 may include a first nucleic acid strand 12 and a second nucleic acid strand 20 which hybridize and form a stem-loop or hairpin structure that places the first reactive moiety 14 in proximity to the second moiety 16. The analyte 18 may bind to the first nucleic acid strand 12 and displace the second nucleic acid strand 20 from the first nucleic acid strand 12, thereby triggering the separation of the first reactive moiety 14 from the second moiety 16 and the change in catalytic activity. In this arrangement, the analyte 18 may be a nucleic acid strand that anneals with the first nucleic acid strand 12. In another embodiment shown in FIG. 4, the first reactive moiety 14 and the second moiety 16 may be attached to separate nucleic acid strands 12 and 20, respectively. The analyte 18 may be a template nucleic acid strand 22 that anneals with the first nucleic acid strand 12 and the second nucleic acid strand 20 to bring the first reactive moiety 14 into proximity to the second moiety 16, thereby triggering the change in catalytic activity.

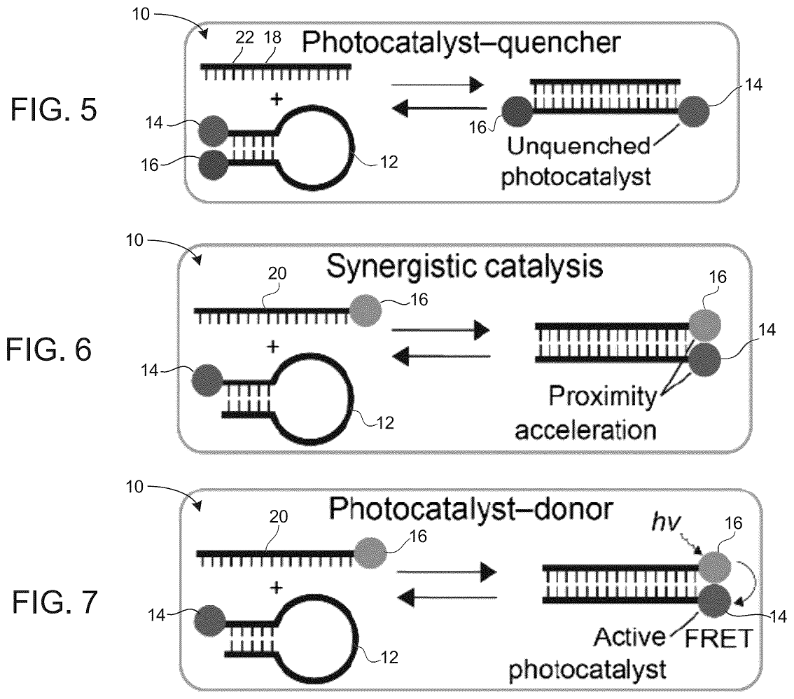

Another embodiment of a switchable catalytic system 10 is shown in FIG. 5. In this arrangement, the first reactive moiety 14 is a photocatalyst and is attached to one end or terminus (5′ or 3′) of the first nucleic acid strand 12, and the second moiety 16 is a quencher of the photocatalyst and is attached to the opposite end or terminus (5′ or 3′) of the first nucleic acid strand 12. The first nucleic acid strand 12 adopts a stem-loop or hairpin structure that places the quencher in proximity to the photocatalyst and inactivates the photocatalyst. The analyte 18 is a template nucleic acid strand 22 that anneals with the first nucleic acid strand 12 and opens the stem-loop structure to move the quencher away from the photocatalyst and activate the catalytic activity of the system.

Another embodiment of the switchable catalytic system 10 is shown in FIG. 6. In this embodiment, the system 10 is a synergistic catalytic system whereby the first reactive moiety 14 and the second moiety 16 synergistically catalyze a reaction when in proximity to each other. The first reactive moiety 14 is attached to the first nucleic acid strand 12 which adopts a stem-loop or hairpin conformation, and the second moiety 16 is attached to the second nucleic acid strand 20 (or trigger strand) which anneals with the first nucleic acid strand 12 to open the hairpin and provide a nucleic acid structure (e.g., DNA duplex) which positions the first reactive moiety 14 in proximity to the second moiety 16 and triggers catalysis.

In an embodiment of the switchable catalytic system 10 shown in FIG. 7, the first reactive moiety 14 is a photocatalyst and the second moiety 16 is a Förster resonance energy transfer (FRET) donor to the photocatalyst. The photocatalyst is attached to the first nucleic acid strand 12 which adopts a hairpin structure, and the FRET donor is attached to the second nucleic acid strand 20 (or trigger strand). The second nucleic acid strand 20 anneals with the first nucleic acid strand 12 to open the hairpin and provide a nucleic acid structure (e.g., DNA duplex) which places the photocatalyst in proximity to the FRET donor. The system 10 may be excited at a wavelength that excites the FRET donor but not the photocatalyst. When the second nucleic acid strand 20 anneals to the first nucleic acid strand 12, the FRET donor is close enough to transfer energy via FRET to the photocatalyst, thereby activating catalytic activity.

In one embodiment, the catalyst includes the first nucleic acid strand which adopts a hairpin or stem-loop conformation. The first reactive moiety is a photocatalyst and the second moiety 16 is a FRET quencher (“Q”) of the photocatalyst. The photocatalyst and the FRET quencher are tethered to respective 5′ and 3′ ends (or 3′ and 5′ ends) of the nucleic acid strand. In this example, the photocatalyst may be a ruthenium complex in which a Ru′ ion is coordinated to two bipyridine ligands and one phenanthroline ligand, and the FRET quencher may be Black Hole Quencher 2 (BHQ-2). In some embodiments, the photocatalyst and the FRET quencher may be pre-installed as abiotic groups on a custom commercial nucleic acids oligomer. Upon absorption of light of an appropriate wavelength (˜450 nm), the ruthenium complex may be excited to a higher energy excited state and is capable of donating an electron or accepting an electron from a substrate, leading to conversion of the substrate to a new molecule. When in proximity to the FRET quencher in the hairpin conformation, the excited state of the ruthenium complex is quenched and catalytic activity is suppressed. In the presence of an analyte that binds to the nucleic acid strand, a conformation change is triggered that opens the hairpin and moves the ruthenium complex far from the FRET quencher and activates catalytic activity.

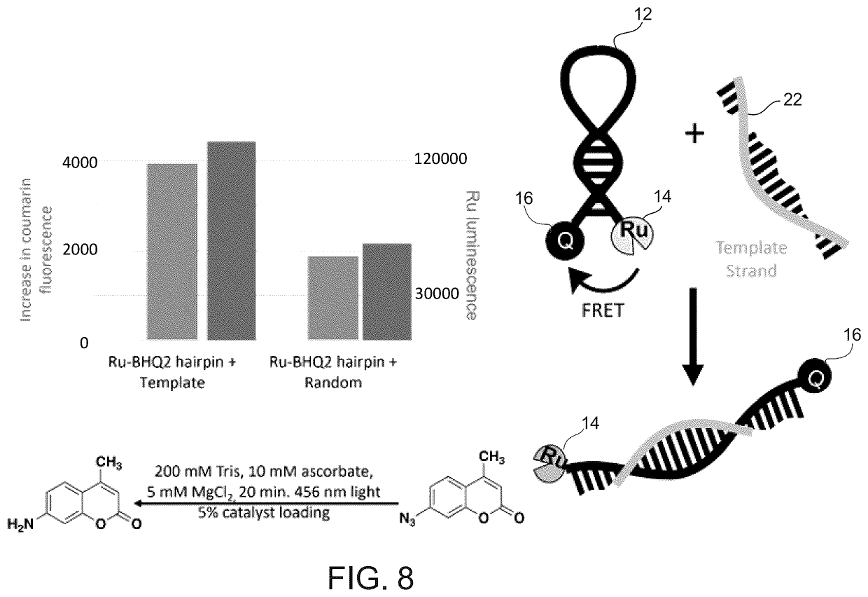

The catalytically active (“on”) and catalytically inactive (“off”) states of the any of the systems described herein having a photocatalyst and a FRET donor or quencher (e.g., FIGS. 5 and 7) may be governed by the distance dependence of FRET (about 10 nm or less (e.g., 0.7 nm to 10 nm, e.g., 0.7 nm, 1 nm, 2 nm, 3 nm, 4 nm, 5 nm, 6 nm, 7 nm, 8 nm, 9 nm, 10 nm). For example, if the second moiety 16 is a FRET quencher, energy transfer and quenching can occur when the photocatalyst and the quencher are within 10 nm of each other. For example, in the arrangements of FIGS. 5 and 8, the moieties 14 and 16 may be within 10 nm of each other in the catalytically inactive (“off”) state and greater than 10 nm away in the catalytically active (“on”) state. On the other hand, if the second moiety is a FRET donor (e.g., FIG. 7), the photocatalyst and the FRET donor may be within 10 nm of each other in the catalytically active (“on”) state and greater than 10 nm away from each other in the catalytically inactive (“off”) state.

An exemplary synthetic scheme for the preparation of the ruthenium complex photocatalyst, and conjugation of the ruthenium complex to DNA is shown in synthetic schemes 8, 9, and 10 in Example 2 below. 1,10-phenanthrolin-5-amine reacted with 4-pentynoic acid in the presence of 4-dimethylaminopyridine (DMAP) and 1-ethyl-3-(3′-dimethylaminopropyl)carbodiimide hydrochloric acid (EDC-HCl). The alkynylated product may be coordinated with cis-dichlorobis(bipyridine)ruthenium (II) (Ru(bpy)₂Cl₂), and then conjugated to an azide-functionalized DNA strand via click chemistry.

Referring to FIG. 8, Applicant has demonstrated product formation upon addition of a template strand 22 complementary to a hairpin nucleic acid strand 12 having a ruthenium complex photocatalyst (first reactive moiety 14) tethered to one terminus and a BHQ-2 quencher (second moiety 16) tethered to the other terminus. Specifically, the addition of the template strand 22 led to formation of the fluorescent product coumarin from an azide precursor relative to addition of a random nucleic acid strand. The hairpin nucleic acid strand 12 opens and anneals with the template strand 22, thereby increasing distance between the ruthenium complex and the quencher and the catalytic activity of the ruthenium complex.

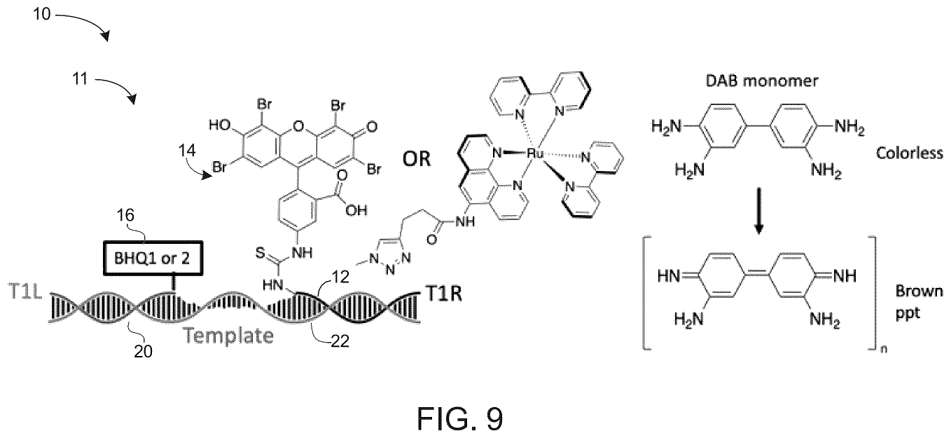

In some embodiments, polymerization of 3,-3′-diaminobenzidine (DAB) may be controlled via a switchable nucleic acid-scaffolded catalytic system of the present disclosure. Polymerization of DAB is a reaction that is useful for generating localized contrast for electron microscopy of biological samples. One exemplary switchable nucleic acid-scaffolded catalytic system 10 for this purpose is shown in FIG. 9. The system 10 includes a first nucleic acid strand 12 tethered at its 3′ end to a photocatalyst as the first reactive moiety 14. The photocatalyst may be an Eosin Y photocatalyst or a ruthenium complex photocatalyst. The system 10 further includes a second nucleic acid strand 20 tethered at its 5′ end to a quencher (BHQ-1 for the Eosin Y photocatalyst or BHQ-2 for the ruthenium photocatalyst). In some embodiments, the oligonucleotides may be purchased with the quenchers pre-installed, and the photocatalysts may be attached using amine coupling or copper-click chemistry. The excited state of the Eosin Y or ruthenium photocatalyst interacts with molecular oxygen and, through electron or energy transfer, affords a reactive oxygen species that induces DAB polymerization. Addition of a template nucleic acid strand 22 that hybridizes to both the first nucleic acid strand 12 and the second nucleic acid strand 20 brings the 5′ end of the quencher-bearing strand in proximity to the 3′ end of the photocatalyst strand. This triggers FRET quenching of the photocatalyst and diminished photocatalytic activity.

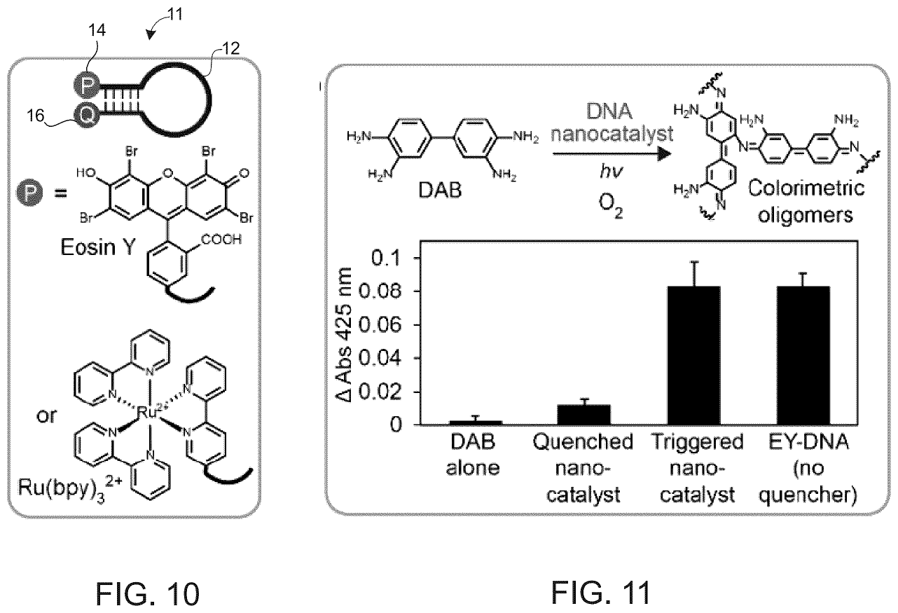

In another arrangement shown in FIG. 10, the catalyst 11 includes a photocatalyst (Eosin Y or ruthenium complex) as the first reactive moiety 14 tethered to one terminus (5′ or 3′) of the nucleic acid strand 12, and a BHQ-1 or BHQ-2 quencher as the second moiety 16 tethered to the other terminus (5′ or 3′) of the same nucleic acid strand 12. In this arrangement, the catalyst 11 is quenched by default in the closed hairpin conformation of the nucleic acid strand 12, and becomes activated when a nucleic acid trigger strand is added that opens the hairpin conformation. Luminescence of the Eosin Y photocatalyst was fully quenched in the closed hairpin and restored upon addition of the complementary trigger strand. The catalytic behavior of the Eosin Y photocatalyst correlated well with its luminescence. Using a colorimetric readout for catalysis, the polymerization of 3,3′-diaminobenzidine (DAB) was detected in the triggered catalyst 11 by increased absorbance at 425 nm corresponding to the polymerized product (see FIG. 11, “triggered nanocatalyst”). Catalytic activity was triggered by addition of a trigger nucleic acid strand complementary to the nucleic acid strand 12. A similar absorbance at 425 nm for polymerized DAB was observed with an Eosin Y-tethered nucleic acid strand without a quencher (see FIG. 11, “EY-DNA (no quencher)”). The switchable DAB polymerization activity of the catalyst 11 opens future opportunities for site-specific contrast in EM imaging.

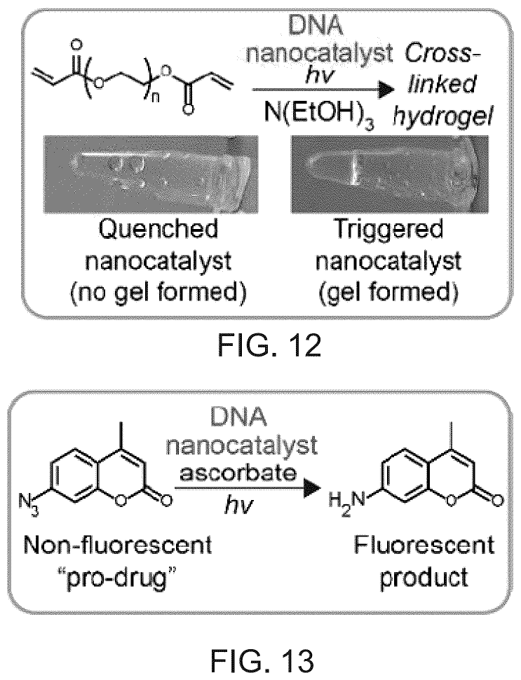

Radical polymerization of the poly(ethylene glycol) diacrylate (PEGDA) monomer was also demonstrated in the triggered catalyst 11 of FIG. 10 (see FIG. 12). The catalyst 11 catalyzed polyacrylate cross-linking to form a hydrogel in the presence of the trigger nucleic acid strand (see FIG. 12). The unquenched photocatalyst was also used to convert an aryl azide prodrug into a fluorescent aniline product (see FIG. 13), opening opportunities for spatially-controlled unmasking of therapeutic compounds.

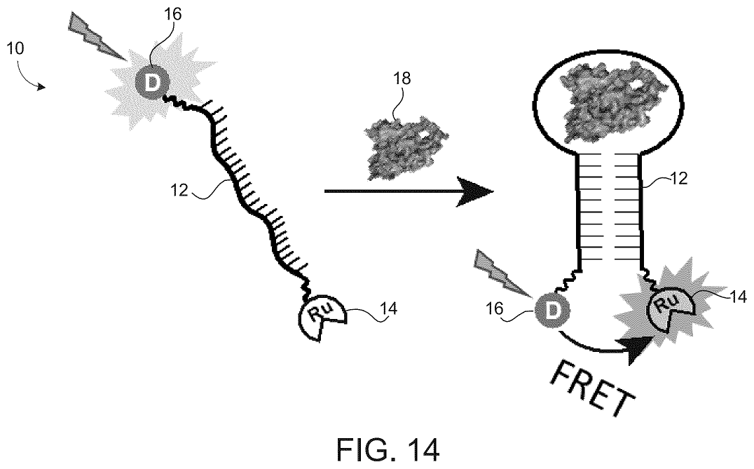

FIG. 14 shows yet another embodiment of the switchable nucleic acid-scaffolded catalytic system 10 whereby a ruthenium catalyst or other photocatalyst (first reactive moiety 14) is appended to one terminus of the nucleic acid strand 12 and a FRET donor (second moiety 16) is appended to the other terminus of the nucleic acid strand 12. Illumination with UV light of a certain wavelength that excites the FRET donor, but does not excite the ruthenium complex, leads to an excited state of the photocatalyst via FRET and activation of catalysis when the photocatalyst and the FRET donor are in proximity. The addition of an analyte 18 triggers a conformational change that shifts the catalytic system 10 from a random conformation in which the FRET donor is separated from the photocatalyst (catalytically inactive or “off” state) to a hairpin conformation in which the FRET donor is in proximity to the photocatalyst (catalytically active or “on” state). In some examples, proximity can be less than 10 nm, preferably about 5 nm or.

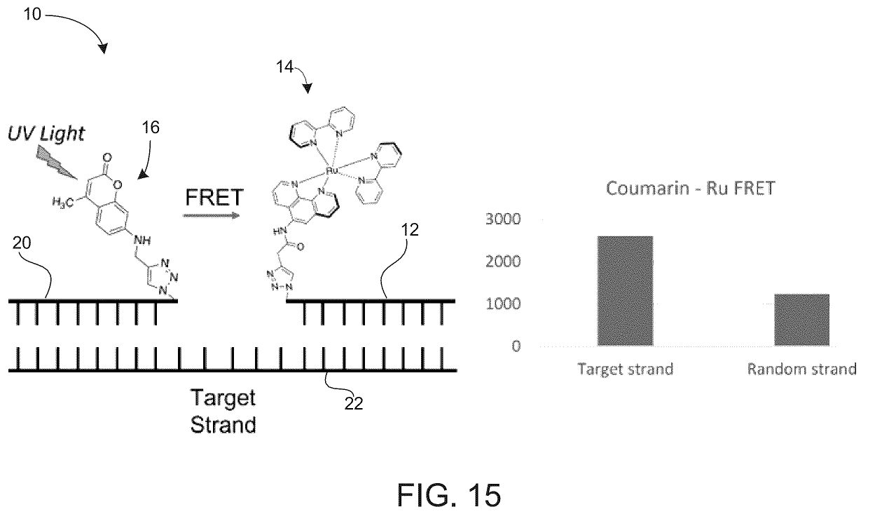

FIG. 15 shows yet another exemplary embodiment of the catalytic system 10 in which the first reactive moiety 14 is a photocatalyst and the second moiety 16 is a FRET donor. In this embodiment, the first nucleic acid strand 12 is appended at one terminus to a ruthenium catalyst (fires reactive moiety 14) and the second nucleic acid strand 20 is appended at one terminus to a coumarin FRET donor (second moiety 16). A trigger nucleic acid strand 22 may be added which is complementary to both of the modified strands 12 and 20. The three strands may anneal, thereby placing the FRET donor in proximity to the ruthenium catalyst and triggering catalytic activity. Applicant has found that addition of a complementary trigger nucleic acid strand 22 (or target strand) to the modified strands 12 and 20 leads to an increase in coumarin-ruthenium FRET compared to a non-complementary random nucleic acid strand (see FIG. 15).

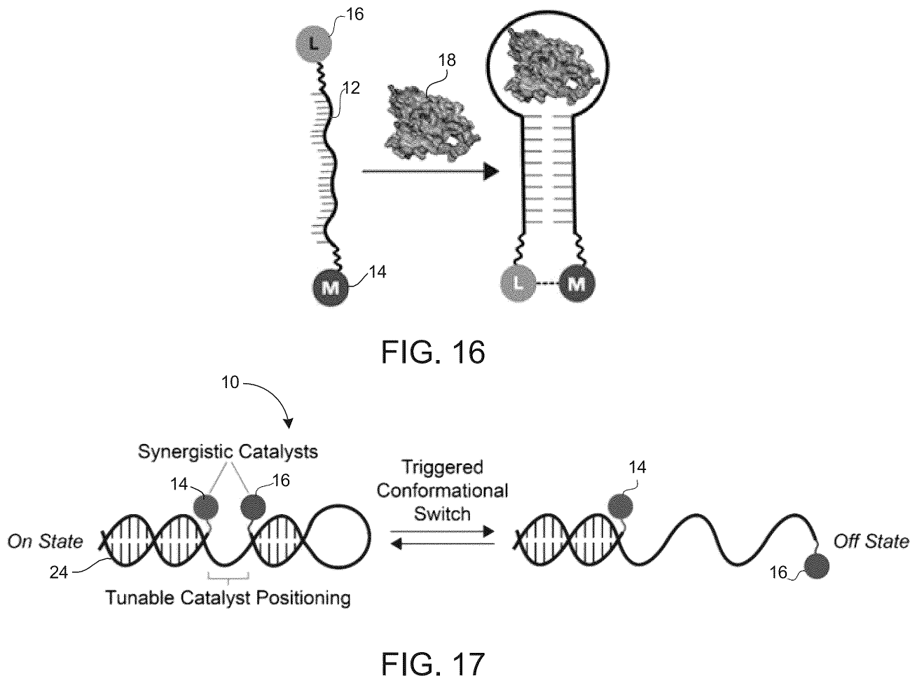

Other possible pairs of reactive moieties for catalytic systems of the present disclosure are shown in FIG. 16 and described below. In an embodiment shown in FIG. 16, the first reactive moiety 14 is a metal complex (“M”) and the second moiety 16 is a ligand (“L”) that binds to the metal and turns on catalytic activity. In a non-limiting example, the metal complex may be a palladium complex that catalyzes a variety of reactions in bio-compatible conditions when in proximity to and activated by the ligand, such as removal of a propargyl group to unmask an amine fluorophore or a therapeutic drug. In another embodiment, the first reactive moiety 14 may be an iridium photocatalyst and the second moiety 16 may be a nickel complex, whereby the iridium photocatalyst and the nickel complex synergistically catalyze a bond-forming reaction when in proximity to each other. In yet another aspect, the moieties 14 and 16 may be a ruthenium photocatalyst and a cobalt catalyst that interact synergistically to convert tetrahydroisoquinoline to isoquinoline.

Another embodiment of the nucleic acid-scaffolded synergistic catalytic system 10 is shown in FIG. 17. In this aspect, the first and second moieties 14 and 16 are synergistic catalysts or co-catalysts that are tethered site-specifically to a nucleic acid scaffold 24 with tunable catalyst spacings. Inter-catalyst spacing and, as a result, catalytic reaction rates may be altered through conformational changes in the nucleic acid scaffold triggered by chemical stimuli, such as analyte binding. The nucleic acid-scaffolded synergistic catalytic system offers multiple advantages including: 1) precise positioning of the catalysts via the programmability of nucleic acid structures and the ease of site-specific modification, 2) convergent and facile synthesis of many catalyst variants through one-step bioconjugation followed by self-assembly, and 3) allosteric switching using strand displacements.

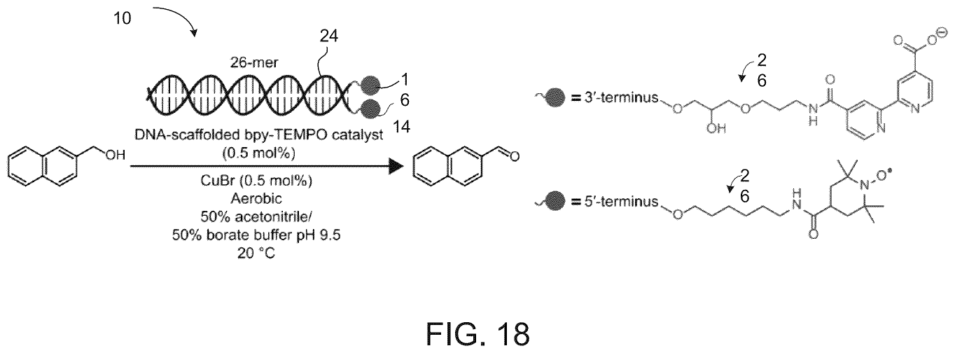

In one example, a nucleic acid-scaffolded synergistic catalytic system 10 for alcohol oxidation is shown in FIG. 18. In this arrangement, the nucleic acid scaffold 24 for the reactive moieties 14 and 16 is a DNA duplex (formed from SEQ ID NO: 5 and SEQ ID NO: 6). The first reactive moiety 14 is a 2,2,6,6-tetramethylpiperidiny-1-yl)oxyl (TEMPO) organic radical, and the second moiety 16 is a bypridine-coordinated copper catalyst. The TEMPO radical is tethered to the 5′ terminus of one of the nucleic acid strands (SEQ ID NO: 6) of the DNA duplex, and the bypyridine (bpy) ligand is tethered to the 3′ terminus of the other nucleic acid strand (SEQ ID NO: 5) of the DNA duplex. In other embodiments, the TEMPO radical may be tethered to the 3′ terminus, and the bpy ligand may be tethered to the 5′ terminus. Sufficiently long tethers 26 were used for attachment to the nucleic acid strands to span the 2 nanometer (nm) diameter of B-form DNA. To prepare the catalytic system 10, a carboxylate-bearing stable nitroxyl radical (4-carboxy-TEMPO or “cTEMPO”) and a carboxylate-bearing bipyridine ligand (4,4,′-dicarboxy-2,2′-bipyridine or “dcbpy”) were bioconjugated to two separate amine-bearing DNA oligonucleotides with complementary 26-nucleotide sequences (SEQ ID NO: 5 and SEQ ID NO: 6). Copper was added immediately prior to the catalytic reactions to metalate the bpy ligand. 2-Napthalenemethanol was oxidized to an aldehyde in the presence of the functionalized DNA duplex and copper (see FIG. 18).

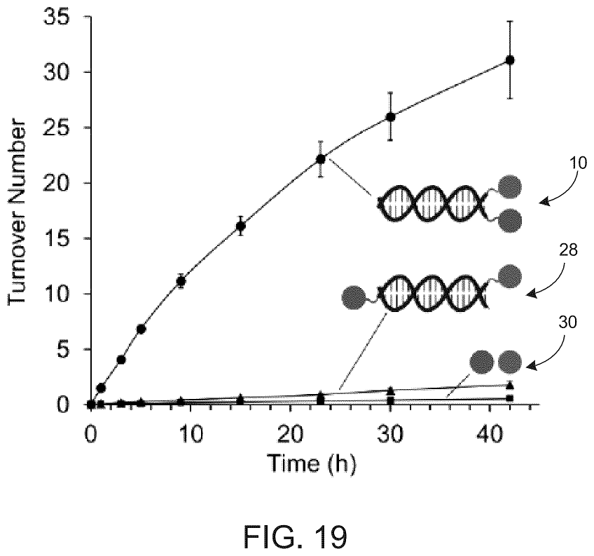

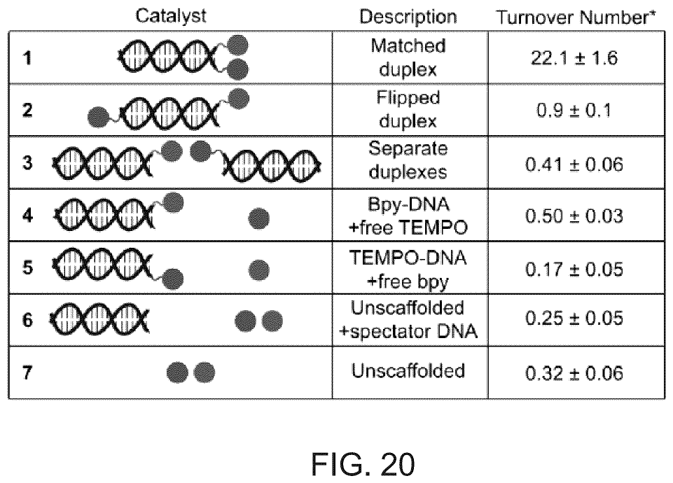

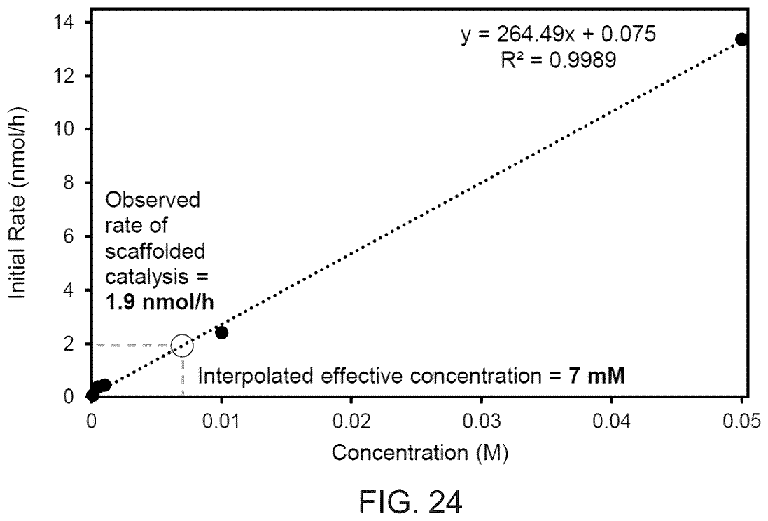

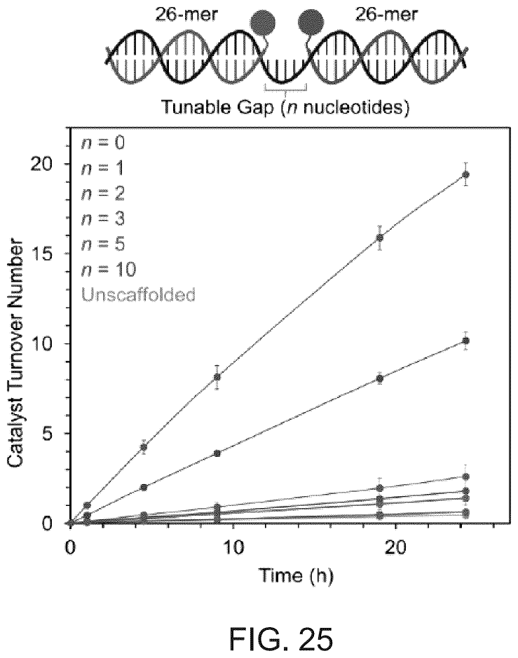

As demonstrated in Example 1, and referring to FIG. 19, alcohol oxidation was measured for the DNA-scaffolded copper-TEMPO catalytic system 10 (DNA duplex scaffold formed from SEQ ID NO: 5 and SEQ ID NO: 6), a DNA scaffold with the two catalysts tethered to opposite 5′ ends (represented by reference 28; DNA duplex scaffold formed with SEQ ID NO: 6 and SEQ ID NO:7), and the unscaffolded catalytic system (represented by reference 30). At 30 hours, the scaffolded reaction showed a 70-fold enhancement in catalyst turnover number (TON). Tethering the co-catalysts to opposite ends of the DNA duplex (˜9 nm distance) resulted in a similar TON to the unscaffolded reaction, indicating that proximity of the co-catalysts is essential for enhanced activity. FIG. 20 shows the increase in catalyst TON for 2-napthalenemethanol oxidation at 23 hours for the catalytic system 10 (“matched duplex” formed with SEQ ID NO: 5 and SEQ ID NO: 6; entry 1) relative to negative control catalysts including a flipped duplex (entry 2; formed with SEQ ID NO: 6 and SEQ ID NO: 7), separate duplexes (entry 3; one duplex formed with SEQ ID NO: 5 and SEQ ID NO: 2 and one duplex formed with SEQ ID NO: 1 and SEQ ID NO: 6), a bpy-DNA conjugate and free TEMPO (entry 4; DNA duplex formed with SEQ ID NO: 5 and SEQ ID NO: 2), a TEMPO-DNA conjugate and free bpy (entry 5; DNA duplex formed with SEQ ID NO: 1 and SEQ ID NO: 6), unscaffolded catalysts with spectator DNA (entry 6; DNA duplex formed with SEQ ID NO: 1 and SEQ ID NO: 2), and unscaffolded catalysts (entry 7). While the reactions depicted in FIGS. 24-25 were performed in a semi-aqueous solvent system optimized from a panel of water/acetonitrile mixtures (1:1 mixtures of acetonitrile and aqueous buffer), DNA-scaffold-dependent catalytic activity enhancement was also observed under fully aqueous conditions, although the turnover number was significantly reduced.

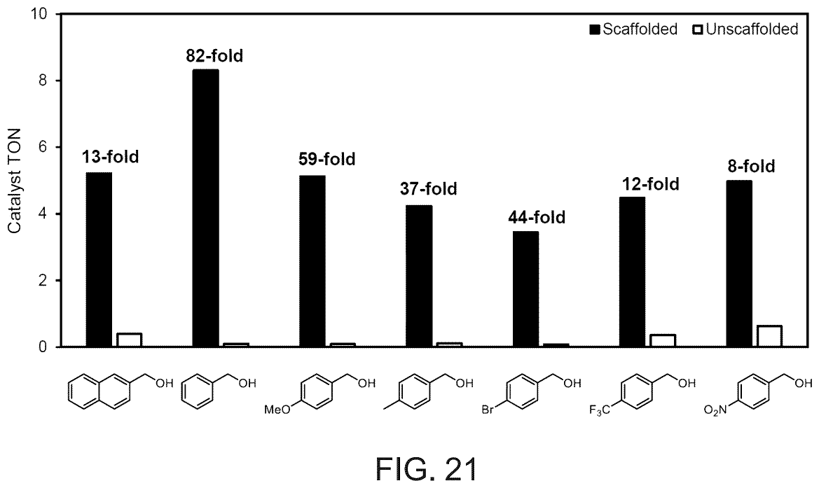

The above oxidation rate acceleration was not specific to 2-napthalenemethanol; it also was observed for six other benzyl alcohol derivatives (see FIG. 21). FIG. 21 shows a comparison of catalyst TONs for nucleic-acid scaffolded (DNA duplex scaffold formed with SEQ ID NO: 5 and SEQ ID NO: 6) and unscaffolded oxidation of various benzylic alcohols. The scaffolding effect led to 8- to 82-fold enhancements in TONs for oxidation of the various benzylic alcohol substrates.

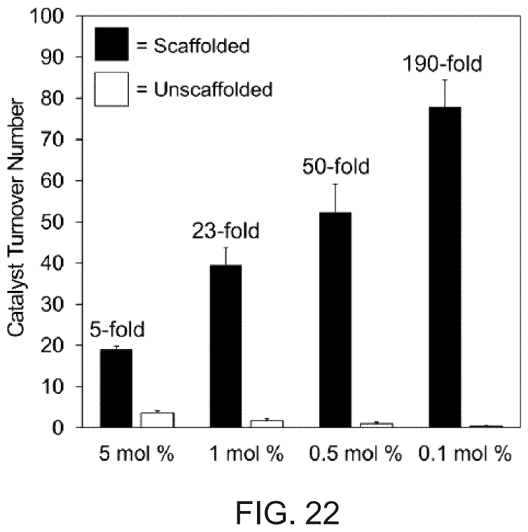

To further confirm that the nucleic acid-scaffolded synergistic catalytic system 10 operates through an intramolecular reaction mechanism (i.e., copper and TEMPO interact on the same DNA helix), the TONs for scaffolded (DNA duplex scaffold formed with SEQ ID NO: 5 and SEQ ID NO: 6) and unscaffolded 2-napthalenemethanol oxidation reactions at different catalyst loadings were compared (see FIG. 22). The TON of the unscaffolded reaction decreased as the catalysts were diluted, presumably due to diminished frequency of synergistic catalyst-catalyst interactions. In contrast, the TON of the scaffolded reaction increased, apparently insensitive to the change in absolute catalyst concentrations.

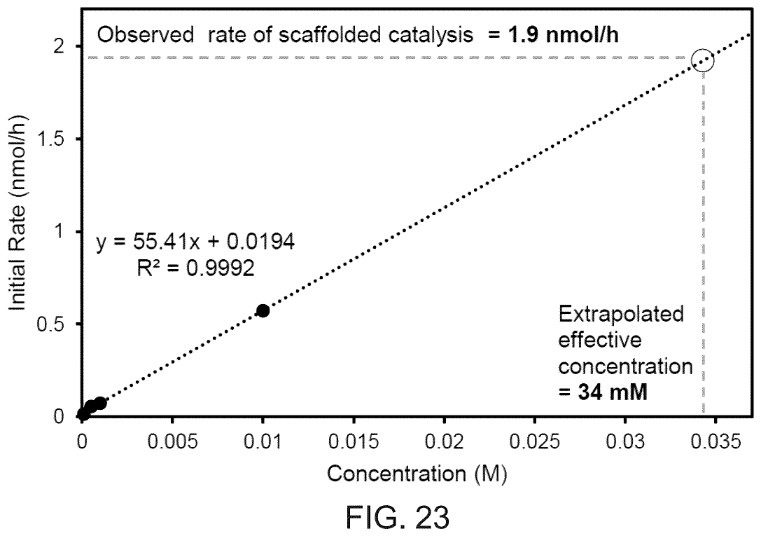

The increase in effective concentration of the TEMPO radical and bpy-Cu catalyst provided by the scaffolded system was estimated experimentally. Initial rates of alcohol oxidation by a single catalyst tethered to DNA in the presence of non-tethered co-catalyst were measured, reasoning that a sufficiently high concentration of non-tethered co-catalyst could mimic the high local concentration of the DNA-tethered co-catalyst. It was found that 50 micromolar (□M) of the nucleic acid-scaffolded Cu-TEMPO catalyst exhibited a reaction rate equivalent to that expected for 34 mM of the non-tethered TEMPO catalyst (FIG. 23) and 7 millimolar (mM) of the non-tethered bpy-Cu complex (FIG. 24). These results suggest that nucleic acid scaffolding provides an increase in effective concentration of over two orders of magnitude. The experimental results match well with a simple geometric model, from which an effective concentration of approximately 13 mM of the bpy-Cu and TEMPO catalysts tethered to the DNA helix was calculated.

A key advantage of DNA as a scaffold for synergistic catalysis is the ability to control the relative spacing of the catalysts using templating strands which facilitate modular assembly of 2D and 3D architectures. As demonstrated in the Examples, this advantage for Cu-TEMPO synergistic catalysis was explored by annealing DNA-catalyst conjugates (SEQ ID NO: 8 and SEQ ID NO: 6) to a series of unmodified templating strands with varying single-stranded spacers controlling the inter-catalyst distance (FIG. 25). Polythymidine spacers were used to prevent the formation of unwanted secondary structures. The architecture with no gap between the co-catalysts (n=0; formed using templating strand with SEQ ID NO: 9) exhibited the fastest rate, and as the length of the intervening spacer region increased, the rate of alcohol oxidation steadily decreased until it matched that of the unscaffolded reaction (see FIG. 25). Templating strand with SEQ ID NO: 10 has a thymidine spacer of 1 (n=1), templating strand with SEQ ID NO: 11 has a thymidine spacer of 2 (n=2), templating strand with SEQ ID NO: 12 has a thymidine spacer of 3 (n=3), templating strand with SEQ ID NO: 13 has a thymidine spacer of 5 (n=5), and templating strand with SEQ ID NO: 14 has a thymidine spacer of 10 (n=10). As can be seen from FIG. 25, gaps of 5 nucleotides or more (n=5) diminished the scaffolding effect considerably.

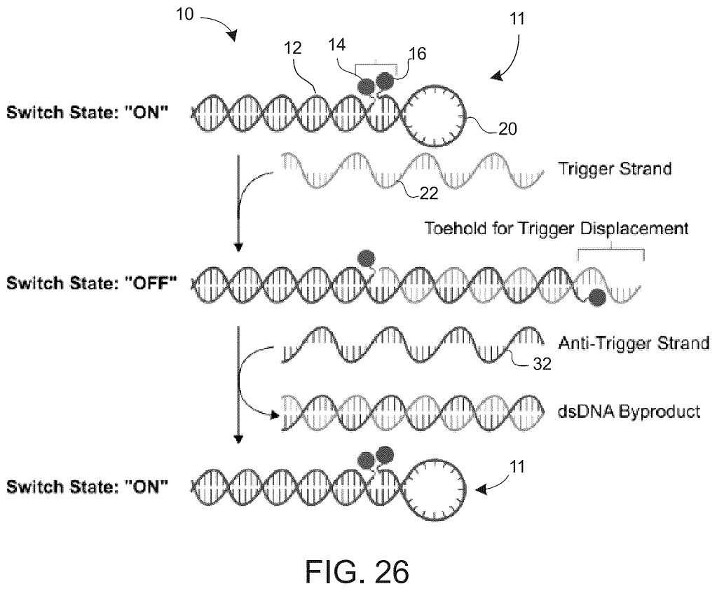

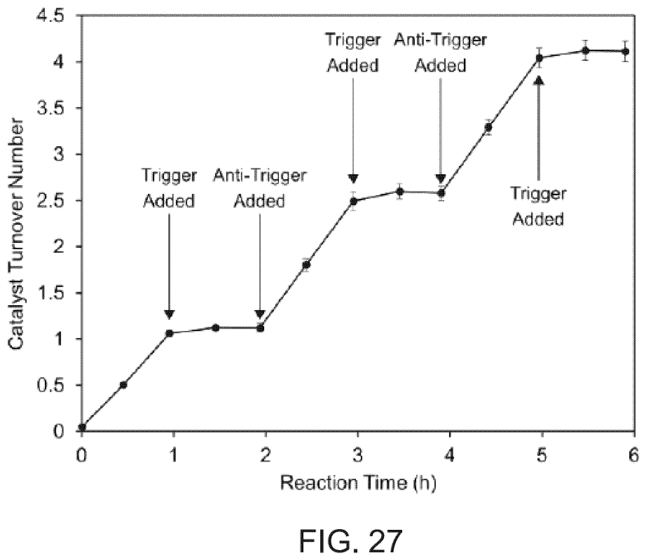

Another embodiment of the switchable nucleic-acid scaffolded synergistic catalytic system 10 is shown in FIG. 26. The system 10 includes a nucleic acid scaffold 24 having a first nucleic acid strand 12 tethered to carboxybipyridine (cbpy) (first reactive moiety 14) which coordinates copper, and a second nucleic acid strand 20 tethered to TEMPO (second moiety 16), whereby the first nucleic acid strand 12 and the second nucleic acid strand 20 hybridize and form a duplex. The second nucleic acid strand 20 includes a stem-loop motif in its sequence. In the presence of a trigger nucleic acid strand 22 complimentary to the stem-loop motif, the structure opens and the co-catalysts are separated, turning off synergistic catalysis (switch state: “off”). Addition of a corresponding anti-trigger strand 32 that recognizes an 8-nucleotide toehold region on the trigger strand 22 displaces the trigger strand 22 from the catalyst 11. As a result, the scaffolded catalyst 11 refolds to its original conformation and catalytic activity is restored (switch state: “on”). As shown in FIG. 27, adding the trigger strand 22 to the reaction solution after 1 hour completely turned off the catalytic oxidation of 2-napthalenemethanol, and catalytic activity was fully restored upon addition of the anti-trigger strand 32. Catalytic performance was unaffected after 3 cycles of an in situ activation/deactivation over 6 hours. These results represent the first demonstration of switchable synergistic catalysis through tuning of the geometric relationship between co-catalysts. Furthermore, this nucleic acid scaffolded catalytic system exhibited activity in the oxidation of the fluorogenic probe 6-methoxy-2-napthalenemethanol demonstrating that switchable catalytic activity can be coupled to a fluorescent output.

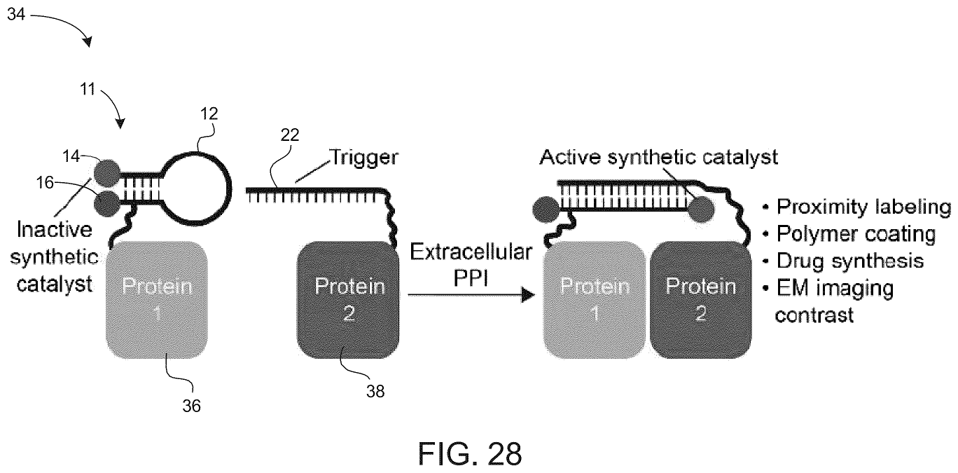

FIG. 28 shows an exemplary system 34 for detecting a protein-protein interaction (PPI) using a switchable nucleic acid-scaffolded catalyst 11. In this system, the nucleic acid-scaffolded catalyst 11 adopts a catalytically inactive conformation, but when the catalyst 11 and a trigger nucleic acid strand 22 are appended to two proteins of interest (a first protein 36 and a second protein 38), the occurrence of a PPI brings the trigger strand 22 close to the catalyst 11 causing a conformational change that activates catalysis. Tethered to the first protein 36 is the catalyst 11 which includes a nucleic acid strand 12 attached to the first reactive moiety 14 at a first end and the second moiety 16 at a second end. The first nucleic acid strand 12 may adopt a stem-loop or hairpin structure which places the first reactive moiety 14 in proximity to the second moiety 16, thereby inactivating the catalytic activity of the first reactive moiety 14. The trigger nucleic acid strand 22 that is complementary to the first nucleic acid strand 12 is tethered to the second protein 38. The occurrence of a PPI triggers the catalytic activity of the first reactive moiety 14 by increasing the distance between the first reactive moiety 14 and the second moiety 16. The triggered catalytic activity may be used to signal the PPI. In some embodiments, the first reactive moiety 14 may be a photocatalyst and the second moiety 16 may be a quencher (e.g., FRET quencher) of the photocatalyst. In alternative embodiments, the first reactive moiety 14 may be a photocatalyst and the second moiety 16 may be a FRET donor which activates catalytic activity when in proximity to the photocatalyst. In yet other arrangements, the first reactive moiety 14 and the second moiety 16 may be co-catalysts which synergistically catalyze a reaction. In the latter arrangements, opening of the hairpin structure upon occurrence of the PPI causes a decrease in catalytic activity. In some embodiments, activation of the catalyst 11 may generate contrast for EM imaging via DAB polymerization.

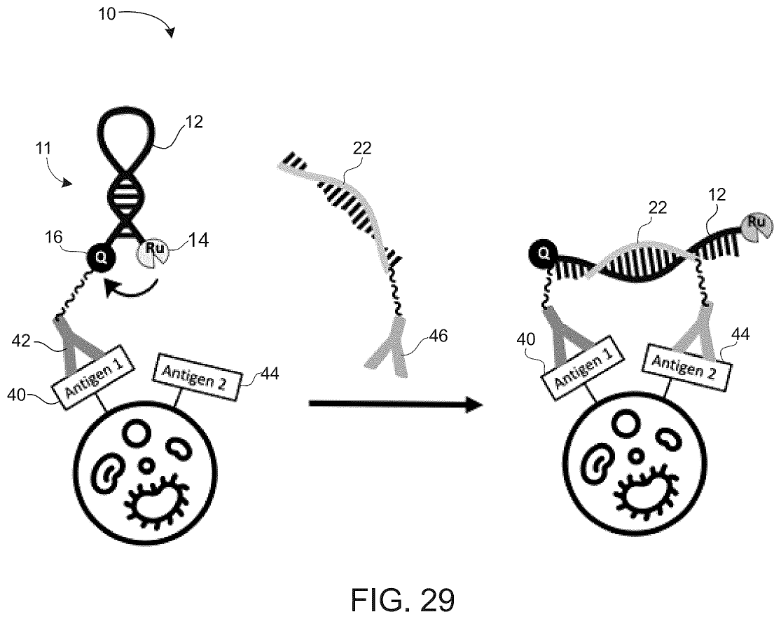

In another aspect, a change in catalytic activity of the catalytic system 10 is dependent on the presence of two cell surface antigens. In such a system depicted in FIG. 29, the catalyst 11 includes the first nucleic acid strand 12 having a stem-loop conformation that is attached to a ruthenium photocatalyst (first reactive moiety 14) at one end and a quencher (second moiety 16) at the other end. The catalyst 11 is recruited to a first cell surface antigen 40 through covalent linkage to a first antibody 42 that is specific for the first antigen 40. The trigger nucleic acid strand 22 is recruited to a second cell surface antigen 44 through covalent linkage to a second antibody 46 that is specific for the second antigen 44. An interaction or proximity between the first and second antigens 40 and 44 causes the trigger strand 22 to bind to the first nucleic acid strand 12 and trigger catalytic activity by opening of the stem-loop structure. In some embodiments, the activated catalyst 11 may generate contrast for EM imaging via polymerization of DAB.

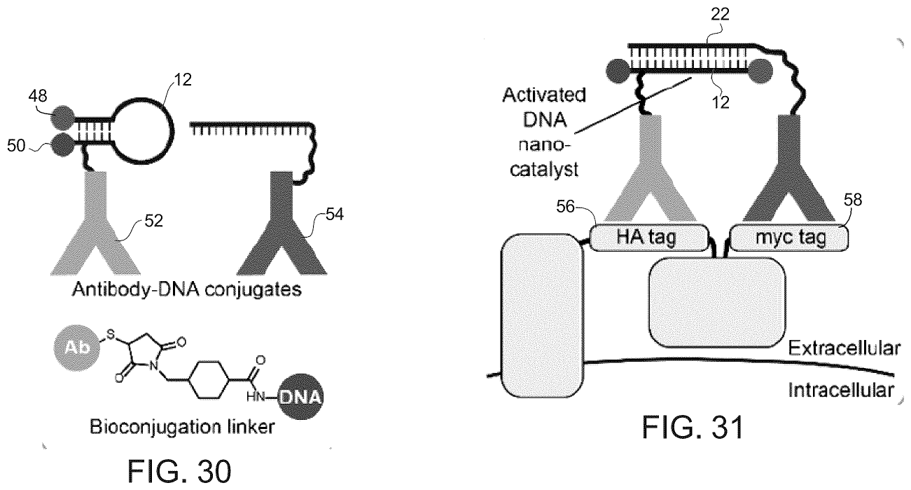

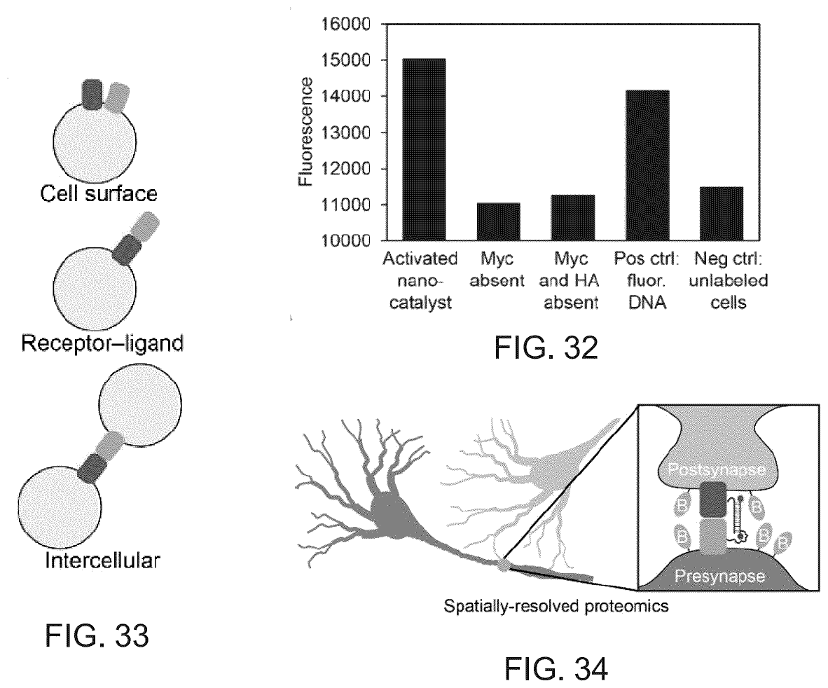

A demonstration of proximity-driven activation of a nucleic acid-scaffolded system on the surface of living yeast cells is shown in FIGS. 30-32. In this embodiment, the scaffolded system includes the first nucleic acid strand 12 having a stem-loop conformation with a fluorophore 48 attached at one end and a quencher 50 of the fluorophore attached at the other end. The first nucleic acid strand 12 and the trigger nucleic acid strand 22 were attached to first and second antibodies 52 and 54 specific for human influenza hemagglutinin (HA) and myc epitope tags, respectively. The nucleic acid strands 12 and 22 were first attached to a bifunctional N-hydroxysuccinimide (NHS) ester-maleimide small molecule via an amine handle on the nucleic acids 12 and 22. Next, the antibodies 52 and 54 that had been pre-reduced to expose reactive cysteine thiols were separately added to the modified nucleic acid strands and to form the nucleic acid-antibody conjugates (see FIG. 30). Yeast (S. cerevisiae) cells were transformed with a protein that displays the human influenza hemagglutinin (HA) and myc epitope tags (56 and 58, respectively) adjacent to one another extracellularly. The nucleic acid-antibody conjugates were introduced to the cells, leading to the recruitment of the nucleic acid-scaffolded fluorophore to the HA tag 56 and the trigger nucleic acid strand 22 to the myc tag 58 (see FIG. 31). The proximity-activated system exhibited a substantial fluorescence increase relative to unlabeled yeast indicating opening of the stem-loop structure (see FIG. 32; “activated nanocatalyst”). The fluorescence increase was similar to the fluorescence observed in positive control yeast stained with a DNA-fluorophore conjugate (see FIG. 32; “pos ctrl: fluor. DNA”). The fluorescence increase was completely dependent on the recruitment of the trigger strand 22 to the myc tag, indicating that the nucleic acid-scaffolded system remains properly quenched on the surface of living yeast in the absence of the myc tag (see FIG. 32).

FIG. 33 shows exemplary classes of PPIs that may be studied using the switchable nucleic acid-scaffolded catalysts of the present disclosure. The switchable catalysts may be used to probe cell surface PPIs, receptor-ligand interactions on cell surfaces, and intercellular PPIs. For instance, in some embodiments, the switchable nucleic acid-scaffolded catalysts of the present disclosure may be activated/deactivated by interacting proteins in a synaptic cleft between neurons (see FIG. 34).

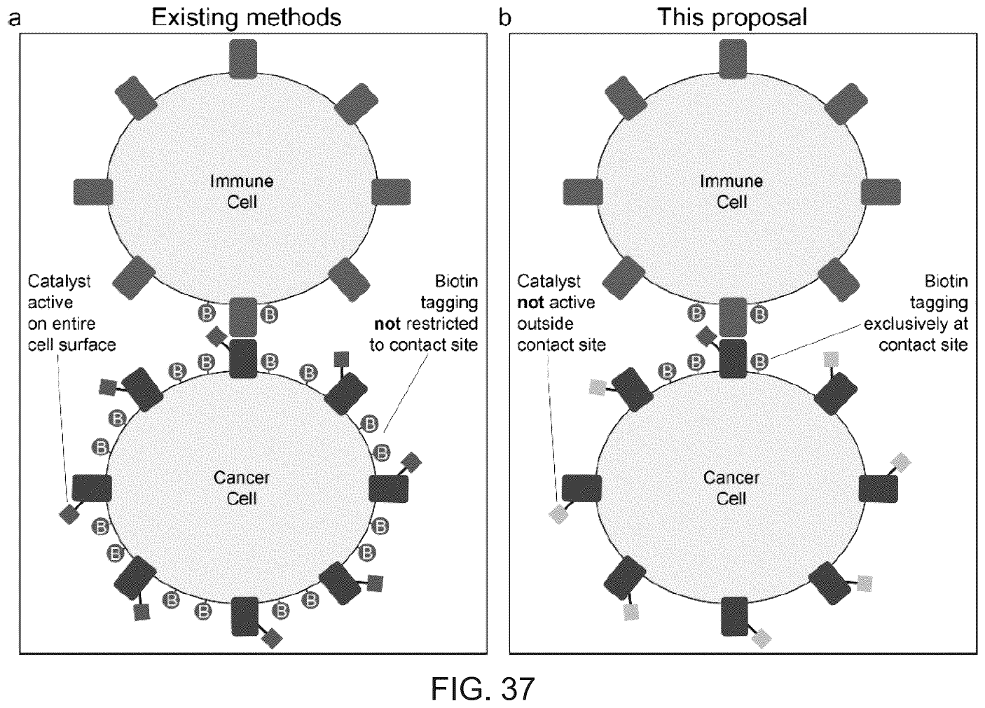

In another aspect, the disclosure the switchable nucleic acid-scaffolded catalyst of the present disclosure may be used for studying and targeting cancer cell—immune cell contacts, based on the use of switchable proximity labeling catalysts that activate only at contact sites of interest. Specific protein-protein interactions at contact sites between cancer cells and immune cells, as well as contacts between different types of immune cells (e.g. the immunological synapse), mediate cancer immune response and evasion. For example, the interaction between programmed death receptor 1 (PD-1) and its ligand (PD-L1) is implicated in suppression of T cell activity and acceleration of tumor progression. 4 In another example, a variety of protein-protein interactions at contact sites between NK cells and cancer influence whether the NK cells are activated or inhibited in their immune response. The switchable catalyst described herein can be used to study and understand the mechanisms of cancer immune cell evasionby determining proteins mediating the evasion process. To develop such a “proteomic list,” one promising approach is to use proximity labeling catalysts for spatially restricted proteomic mapping. In these methods, a catalyst—such as a peroxidase (APEX or HRP) or a photocatalyst—is tethered to a synaptic protein of interest, and a small molecule probe, consisting of a substrate for the catalyst tethered to biotin, is introduced. The catalyst converts the substrate into a reactive species that bonds covalently with nearby proteins, but only within a tight labeling radius, owing to rapid quenching of the reactive species. Nearby proteins are thus tagged with biotin, which is followed by lysis, pull-down using streptavidin, and identification of the proteome using high-resolution mass spectrometry. Despite the promise of proximity labeling approaches, the specificity of labeling at cell-cell contacts is intrinsically limited because the protein to which the catalyst is appended may not be localized exclusively at the contact sites in the methods used in the art (FIG. 37A). Furthermore, individual cell-cell contacts may vary based on the identity of intercellular protein binding partners and therefore cannot be differentiated.

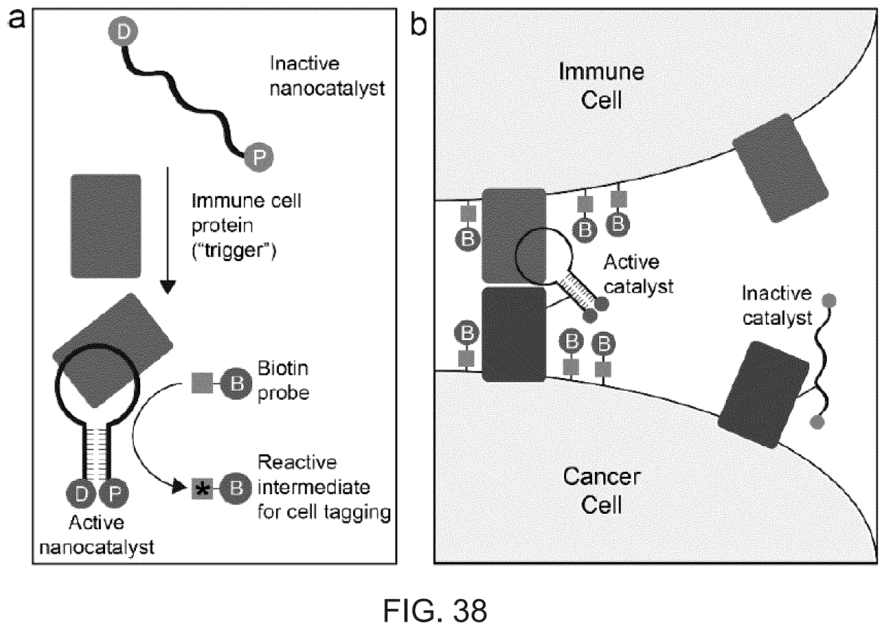

The switchable catalyst of the present invention are able to overcome the shortcomings of the prior art by being “smart” proximity labeling catalysts that activate selectively at cell-cell contacts (FIG. 37B). The present switchable catalysts can enhance the specificity of proteomics at immune cell-cancer cell contacts for proximity tagging. The DNA nanocatalyst is appended to a cancer cell surface protein, and it will be “switched off” by default. The catalyst will only “turn on” when it encounters a second protein that resides on the surface of a neighboring cell, such as an immune cell. These switchable nanocatalysts will enable proximity biotin labeling with unprecedented specificity because they can be designed to activate at the site of interaction between two user-defined proteins. The switchable DNA nanocatalysts consist of a synthetic photocatalyst attached to a conformationally-responsive DNA aptamer (FIG. 38). Upon binding to a specific protein, the DNA aptamer switches its conformation, bringing a FRET donor (“D”) and the photocatalyst (“P”) into proximity, thus activating a reaction in which a precursor biotin substrate (such as an azirine) is converted to a highly reactive carbene or nitrene. These reactive intermediates enable proximity labeling within a tight radius, owing to the rapid quenching of carbenes in aqueous solution. 6 Importantly, the switchable DNA nanocatalysts can be configured to respond not only to proteins, but also chemically diverse analytes, including small molecules, ions, glycans, and reactive oxygen species.

The switchable catalysts can be used in both in vitro proximity labeling of purified proteins (e.g., biotin tagging using imaging and Western blot assays) and targeting the DNA nanocatalysts to the surface of cancer cell lines through attachment to a genetically encoded recombinant protein. Activity of the nanocatalysts for spatially-restricted biotin tagging will de determined and verified that catalyst activation is dependent on the presence of the “trigger” protein. For example, mixed cell culture assays, can be used in which one cell line will be coated with the DNA nanocatalyst, and the other will be coated with the “trigger” protein that activates the nanocatalyst. The proximity biotin labeling substrate will be added into this mixed culture, performing the photocatalytic reaction, and images can be gathered as to whether biotin tagging occurs exclusively at the intended cell-cell contact sites.

In some aspects, the switchable nucleic acid-scaffolded catalysts may be used for proximity labeling at extracellular PPIs, whereby catalyst activation triggered by the conformational change leads to conversion of a substrate into a reactive intermediate which promiscuously tags endogenous proteins within a defined labeling radius. Using nucleic acid-scaffolded catalysts for this purpose includes advantages such as the ability to fine-tune the nucleic acid constructs to ensure sensitive reconstitution while avoiding perturbation of the PPIs, and the availability of multiple synthetic photocatalysts and proximity labeling chemistries that can be used in the platform.

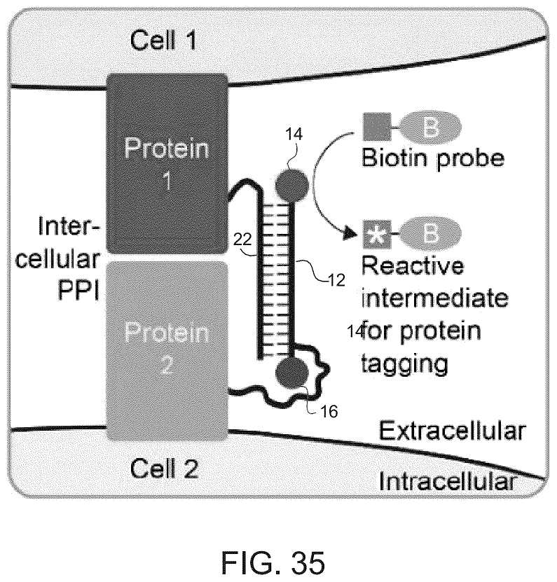

An exemplary application of the switchable nucleic acid-scaffolded catalysts for proximity labeling at intercellular PPIs is shown in FIG. 35. In the embodiment of FIG. 35, the first nucleic acid strand 12 of the catalyst bears a photocatalyst (e.g., an iridium photocatalyst, a ruthenium photocatalyst (Ru(bpy)₃²⁺)) as the first reactive moiety 14, and a FRET quencher as the second moiety 16. The FRET quencher is selected based on spectral overlap with the emission spectrum of the photocatalyst. The first nucleic acid strand 12 is tethered to a surface extracellular protein of a cell, and the trigger nucleic acid strand 22 is tethered to a surface extracellular protein of a neighboring cell. The occurrence of a PPI between the proteins leads to hybridization of the trigger strand 22 to the first nucleic acid strand 12, opening of the hairpin structure of the first nucleic acid strand 12, and triggered catalytic activity of the first reactive moiety 14. If an iridium photocatalyst is used, activation may lead to generation of a highly reactive carbene with a short labeling radius. Other photocatalysts may convert aryl azides to nitrenes or may generate phenoxyl radicals for protein tagging. Biotin-tethered proximity labeling probes may be used, and then stained with streptavidin to visualize labeled contact sites by fluorescent microscopy.

In another aspect of proximity labeling using the switchable catalysts, a split invader system may be used. Split invader systems include two “split invader” oligos, with each split invader oligo attached to a DNA aptamer to recruit a specific epitope. A “full invader” may form when two split invaders bind in immediate proximity on a surface (i.e., a cell, a bead, a plastic substrate, a glass substrate, a metallic substrate, a paper substrate, a composite substrate, a ceramic substrate) and anneal to each other. For this activation mechanism, a photocatalyst ssDNA strand and a quencher ssDNA strand may be annealed in a duplex. In the duplex conformation, the photocatalyst is inactive due to its proximity to the quencher. In some embodiments the split invader oligos are attached to an aptamer, antibody, antigen, or protein-protein interactions given in Table 2. In some embodiments the split invader targets interact in a protein-protein interaction. In other embodiments, the split invader targets do interact in a protein-protein interaction.

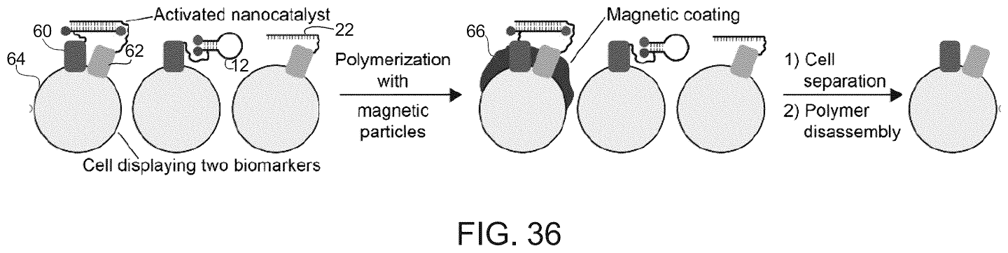

Additionally, the switchable nucleic acid-scaffolded catalysts of the present disclosure may be used for detection and/or isolation of circulating tumor cells (CTCs) that have detached from the primary tumor (for example, see FIG. 36). In one embodiment, as exemplified in FIG. 36, a first and second biomarkers or epitopes 60 and 62 on the surfaces of cells 64 may be conjugated to the first nucleic acid strand 12 and the trigger nucleic acid strand 22, respectively. Only cells having both biomarkers 60 and 62 will exhibit catalyst activation by opening of the stem-loop structure of the first nucleic acid strand 12 with the trigger strand 22. In some aspects, catalyst activation may initiate surface radical polymerization in the presence of magnetic nanoparticles, leading to the synthesis of cross-linked polymers containing magnetic particles trapped inside. The magnetic coating 66 thus formed on the tumor cell may enable isolation of the tumor cell from other cells, and may be removed at the end of the workflow through photodegradation (polymer disassembly). The cell surface biomarkers may or may not engage in a PPI, since cancer proteins on the same cell do not necessarily engage in a PPI. Examples of CTC biomarkers that may be used for detection of CTCs via this approach include, but are not limited to, HER2 and EGFR (breast cancer), HER2 and HER3 (breast cancer), MET and EGFR (lung cancer), EpCAM and PD-L1 (breast cancer), CD44, CD47, and c-met (ovarian cancer), and HER2 and ILI3Rα2 (glioblastoma).

In another aspect, the technology disclosed herein may be included in a kit intended for performing an assay. In some embodiments the assay may perform proximity labeling with previously discussed split invader systems. In some embodiments the assay perform proximity labeling on eukaryotic cells, including mammalian and non-mammalian cells. In other embodiments, the assay may perform proximity labeling on prokaryotic cells.

The assay kit contains all necessary reagents and standards for calibrating said one or more assays and for performing said one or more assays on one or more unknown samples. In one embodiment, the assay may contain at least one photocatalyst ssDNA strand and at least one quencher ssDNA strand annealed in a duplex, at least two split invader oligos with each split invader attached to a target (i.e., an aptamer or antibody) corresponding to at least two suspected antigens or epitopes found in the user-supplied sample. Examples of targets are explored in Example 2 and Table 2.

In some embodiments the assay is a mix-and-read assay (i.e., a homogenous, non-separation assay). In some embodiments the assay is read optically, spectroscopically (i.e., by fluorescence emission, by infrared emission, by ultraviolet emission, or by Raman scattering), or colorimetrically. In some embodiments, the assay may be used for proximity labeling of endogenous proteins within a defined radius of the site of catalyst activation, and the assay kit components may include a proximity labeling substrate such as biotin phenol and the oxidant ammonium persulfate. In some embodiments, the assay may be used for synthesis of cross-linked polymer hydrogels at the site of catalyst activation, and the assay components may include poly(ethylene glycol) diacrylate, triethanol amine, and N-vinylpyrrolidone.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1: DNA-Scaffolded Synergistic Catalysis

Creation of catalysts that mimic enzymes through the pre-organization of multiple reactive groups is a long-standing goal. In natural enzymes, protein scaffolds hold multiple functional groups in proximity, engendering massive rate accelerations that are not observed when the same functional groups are unscaffolded. While matching the sophistication of enzymes has been elusive in synthetic systems, supramolecular enzyme mimics offer a broader palette of functional groups, since they are not limited to the natural amino acids and cofactors. Impressive catalytic properties have been demonstrated in a variety of enzyme-mimicking supramolecular scaffolds.

An exciting area of recent progress in supramolecular catalysis is the pre-organization of synergistic catalytic reactions. Synergistic catalysis combines two co-catalysts to carry out a reaction that would not be possible using either co-catalyst alone. Examples include a variety of synergistic combinations of transition metal catalysts, photocatalysts, organocatalysts, radical catalysts, and Lewis acid catalysts. In scaffolded synergistic catalysis, the proximity afforded by precise positioning of the co-catalysts accelerates the reaction compared to unscaffolded reactions, in which the large mean distance between the co-catalysts limits synergistic interactions, often necessitating high loadings. Successful demonstrations of scaffolded synergistic catalysis have utilized peptoids, peptides, foldamers, soluble proteins, metal—organic frameworks, covalent—organic frameworks, and polymers. However, it remains challenging to fine-tune the spacing of each co-catalyst. Furthermore, the previously demonstrated scaffolds for synergistic catalytic reactions are difficult to engineer for stimuli-triggered conformational changes to alter the rate of the reaction. This important property of enzyme catalysis, allostery, is of fundamental interest for enzyme-mimicking systems and for the development of sensors and responsive materials.

Inventors envisioned that DNA-scaffolded synergistic catalysis would offer multiple advantages: 1) precise positioning of the catalysts due to the programmability of DNA and the ease of site-specific modification, 2) convergent and facile synthesis of many catalyst variants through one-step bioconjugation followed by self-assembly, and 3) allosteric switching using DNA strand displacements (FIG. 1). Indeed, the outstanding advantages of DNA have previously been demonstrated in templating synthetic reactions and influencing the rates of mono-catalytic processes. DNA has been used as a template to accelerate bond formation in a variety of reactions and to bring substrates proximal to catalysts. DNA has also been used as a scaffold to influence mono-catalytic processes for asymmetric synthesis. Conformational switching of DNA has been used to regulate catalytic activity of mono-catalytic gold bound to DNA, to activate heme-catalyzed peroxidation, and to alter the distance between two enzymes in a cascade reaction. However, to the best of our knowledge, DNA-scaffolded synergistic catalysis has not been demonstrated, and more broadly, no scaffolded synergistic catalysis has been performed that incorporates the advantages of DNA (convergent synthesis, precise positioning, and switching in response to stimuli). Here, inventors report DNA-scaffolded Cu/TEMPO oxidation of alcohols. Inventors show that synergistic catalysis can be optimized through precise orientation of the co-catalysts on DNA scaffolds, and activity can be controlled through conformational switching of DNA.

Inventors chose Cu/TEMPO alcohol oxidation as a proof-of-principle reaction to demonstrate DNA scaffold-dependent rate enhancement because of its remarkably mild reaction conditions and the synergistic cooperation of the two co-catalysts. Additionally, Cu/TEMPO oxidations have been accelerated through arranging the two co-catalysts on several different scaffolds, including peptoids, silica particles, and simple synthetic tethers. Inventors envisioned that a DNA scaffold could allow for similar enhancements in reactivity while also allowing highly tunable co-catalyst positioning.

Inventors prepared a simple DNA duplex with the two co-catalysts (Cu and TEMPO) attached to the end of the helix via sufficiently long tethers to span the 2 nm diameter of B-form DNA (FIG. 2A). First, inventors bioconjugated a carboxylate-bearing stable nitroxyl radical (4-carboxy-TEMPO) and a carboxylate-bearing bipyridine ligand (4,4′-dicarboxy-2,2′-bipyridine) to two separate amine-bearing DNA oligonucleotides with complementary 26-nucleotide sequences. The resulting small molecule-DNA conjugates were HPLC purified, and their identities were confirmed using electrospray ionization coupled with high resolution and mass accuracy mass spectrometry along with negative electron transfer dissociation tandem mass spectrometry. The complementary DNA-catalyst conjugates were assembled into a duplex with thermal annealing in the presence of NaCl. Copper was added immediately prior to catalytic reactions to metalate the bipyridine ligand.

Catalytic activity of the scaffolded DNA duplex (0.5 mol %) was evaluated by monitoring oxidation of 2-napthalenemethanol (FIG. 18-20). The activity of the DNA-scaffolded catalyst (condition 1) was dramatically enhanced relative to that of the unscaffolded reaction (condition 7); at 30 h, the scaffolded reaction showed a 70-fold enhancement in catalyst turnover number (TON). Tethering the co-catalysts to opposite ends of the DNA duplex (˜9 nm) resulted in a similar TON to the unscaffolded reaction, indicating that proximity of the co-catalysts was essential for enhanced activity (condition 2). To confirm that the DNA remained structurally undamaged and annealed under reaction conditions, inventors recovered DNA from the reaction solution by alcohol precipitation and analyzed it by agarose gel electrophoresis. The mobility of the DNA was unchanged, and no fragmentation was observed. While the reactions depicted in FIGS. 18-20 were performed in a semi-aqueous solvent system, optimized from a panel of water/acetonitrile mixtures, inventors also observed DNA-scaffold-dependent activity enhancement under fully aqueous conditions, although the TON was significantly reduced.

Control reactions confirmed that scaffold-induced proximity of the co-catalysts was responsible for the observed activity enhancement (FIG. 20). When one co-catalyst was tethered to the DNA duplex and the other was not (conditions 4 and 5), or when the co-catalysts were tethered to separate duplexes (3), the TON was similar to that of the unscaffolded reaction. These results suggest that the activity enhancement observed in condition 1 was not due to an intrinsic increase in the activity of either co-catalyst upon conjugation to DNA. They further suggest that substrate recruitment to the DNA scaffold does not contribute significantly to the activity enhancement observed in 1. The DNA-scaffolded rate acceleration was not specific to 2-napthalenemethanol; it was also observed for six benzyl alcohol derivatives (FIG. 21). During the scaffolded catalyst time-course reactions, inventors observed a gradual decrease in reaction rate (FIG. 20), which inventors determined was not due to product inhibition. Spiking in additional copper after the reaction rate had plateaued increased catalytic activity, suggesting that a catalyst deactivation pathway such as copper aggregation may be operative.

To further confirm that the DNA-scaffolded synergistic co-catalysts operate through an intramolecular reaction mechanism (i.e., Cu and TEMPO interact on the same DNA helix), inventors compared the TONs of the scaffolded and unscaffolded reactions at different catalyst loadings (FIG. 22). The TON of the unscaffolded reaction decreased as the catalysts were diluted, presumably due to diminished frequency of synergistic catalyst—catalyst interactions. In contrast, the TON of the scaffolded reaction increased, apparently insensitive to the change in absolute catalyst concentration. The difference in reactivity was especially pronounced at lower catalyst loadings: at 0.1 mol % loading, the scaffolded catalyst turned over 190 times more than the unscaffolded catalysts.

To further investigate the DNA-scaffolded Cu-TEMPO activity enhancement, inventors estimated the increase in effective concentration experimentally and using a simple theoretical model. Inventors measured the initial rates of alcohol oxidation by a could potentially mimic the high local concentration of the DNA-tethered co-catalyst. Inventors determined that 5011M of the DNA-scaffolded Cu-TEMPO catalyst exhibited a reaction rate equivalent to that expected for 7 mM of the non-tethered dcbpy-Cu complex and 34 mM of the non-tethered TEMPO catalyst (FIGS. 23-24). These results suggest that DNA scaffolding provided an increase in effective concentration of over two orders of magnitude. These experimental results matched well with a simple geometric model, from which inventors calculated an effective concentration of approximately 13 mM of the Cu-bpy and TEMPO catalysts tethered to the DNA helix.

A key advantage of DNA as a scaffold for synergistic catalysis is the ability to control the relative spacing of the catalysts using templating strands, which facilitate modular assembly of 2D and 3D architectures. Inventors explored this advantage for Cu-TEMPO catalysis by annealing DNA-catalyst conjugates to a series of unmodified templating strands, with varying single-stranded spacers controlling the inter-catalyst distance (FIG. 25). Inventors used polythymidine spacers to prevent the formation of unwanted secondary structures, which have been reported in DNA-templated synthesis. The architecture with no gap between the co-catalysts exhibited the fastest rate, and as the length of the intervening spacer region increased, the rate of alcohol oxidation steadily decreased until it matched that of the unscaffolded reaction (FIG. 25). Inventors anticipate that more advanced 2D and 3D scaffolding architectures should enable even finer control over the rate of synergistic catalysis.

Taking inspiration from dynamic strand interchange reactions 61 and the widespread application of FRET-based molecular beacons, inventors envisioned that proximity-dependent synergistic catalysis might be incorporated into a stimulus-responsive switch architecture (FIG. 26). By incorporating a stem-loop motif into the sequence of one of the co-catalyst conjugates, inventors created a dynamic DNA structure to link catalytic activity to sequence recognition. In the presence of a trigger sequence complementary to the stem-loop motif, the structure opens, holding the co-catalysts apart and turning off synergistic catalysis. When a corresponding anti-trigger sequence recognizes an 8-nucleotide toehold region on the trigger strand and displaces the catalytic assembly, the scaffolded catalysts refold, and catalytic activity is restored. Adding the trigger strand to the reaction solution after 1 h completely turned off the catalytic oxidation of 2-napthalenemethanol, and catalytic activity was fully restored upon addition of the anti-trigger (FIG. 27). Catalytic performance was unaffected after 3 cycles of in situ activation/deactivation over 6 h. These results represent the first demonstration of switchable synergistic catalysis through tuning the geometric relationship between co-catalysts. Furthermore, the DNA-scaffolded catalyst exhibited activity in the oxidation of the fluorogenic probe 6-methoxy-2-napthalenemethanol, demonstrating that switchable catalytic activity can be coupled to a fluorescent output. In the future, inventors anticipate that incorporating various recognition elements into the stem-loop of this switchable DNA architecture will allow catalysts to be developed that respond to small molecule or protein stimuli.

In conclusion, inventors have successfully demonstrated the first DNA-scaffolded acceleration of synergistic catalysis, using Cu/TEMPO oxidation of alcohols as a model reaction. The DNA backbone allowed for systematic exploration of the scaffolded reactivity, and inventors' data suggest that the reactivity enhancement is due to the increased effective concentration afforded by proximity of the scaffolded co-catalysts. The DNA backbone allowed for dynamic activity switching using a strand-displacement approach. While this initial demonstration was not intended to pursue a new synthetic method, with further optimization this scaffolding approach may provide solutions to challenges in synergistic catalysis. DNA-scaffolded synergistic catalysis will be a promising strategy to accelerate other synergistic catalytic reactions (beyond Cu-TEMPO oxidation) in which interactions between the two co-catalysts are rate-limiting. This acceleration may allow for low catalyst loadings in reactions that currently require high concentrations of both co-catalysts and may even unlock new reactions that are inaccessible in an unscaffolded format. Furthermore, information-encoding properties of DNA could enable DNA-encoded discovery of synergistic catalysts. Finally, the groundwork laid in this study could be applied to create functional DNA nanomaterials containing synergistic catalytic sites.

Materials

All solvents used in this study were purchased from Sigma Aldrich in HPLC Grade and used without further purification. All reagents were purchased from TCI, Fisher, Sigma Aldrich, Santa Cruz Biotechnology, Alfa Aesar, or Ambeed and used without further purification. Unmodified and amine-modified DNA oligonucleotides were purchased from Integrated DNA Technologies with standard desalting.

HPLC Analysis and Purification

Reverse-phase HPLC analysis and purification of DNA conjugates and reverse-phase HPLC reaction tracking were performed on a Shimadzu Prominence HPLC system with quantification at 254 nm. HPLC analysis and purification of DNA oligonucleotides was performed using a 4.0×300 mm Kromasil 100-5-C18 column with 1.0 mL/min flow under ion-pairing conditions using aqueous 100 mM triethylammonium acetate pH 7.0 (TEAA) as mobile phase A and acetonitrile as mobile phase B (using a gradient from 10-30% B over 10 minutes, followed by a 1 minute ramp to 90% and a 1 minute wash at 90% B and then re-equilibration at 10% B), with a column oven temperature of 50° C. HPLC reaction tracking of alcohol oxidation was performed using a 4.6×50 mm)(Bridge C18 (3.5 μm) column with 1.0 mL/min flow using water with 0.1% trifluoroacetic acid as mobile phase A and acetonitrile as mobile phase B, with a column oven temperature of 30° C. (using a gradient from 10-90% B over 5 minutes, followed by a 1.5 minute wash at 90% B and then re-equilibration at 10% B). The differences in absorbance at 254 nm for each substrate and product were accounted for by construction of calibration curves in triplicate in the linear range of each compound.

NMR Spectroscopy

1H and 13C{1H} NMR spectra were recorded on a Bruker Avance 500 MHz spectrometer. Chemical shifts (δ) are given in parts per million and referenced to TMS.

UV-Vis Spectroscopy

DNA concentrations were determined by UV-Vis spectroscopy (absorption at 260 nm) using a Nanodrop One Spectrophotometer (ThermoFisher Scientific).

Mass Spectrometry

All MS and MS/MS experiments were performed on a quadrupole-Orbitrap-quadrupole linear ion trap (q-OT-QLT) hybrid mass spectrometer system (Orbitrap Fusion Lumos, Thermo Fisher Scientific, San Jose, CA) that was modified to perform negative electron transfer dissociation (NETD). 1 Following purification with ion-exchange HPLC, samples were diluted to 51.1M of ssDNA-catalyst conjugate in 70% acetonitrile and 30% nuclease-free water with 10 mM ammonium acetate. Solutions were pH-adjusted to pH 9.5 with ammonium hydroxide. The ssDNA-catalyst conjugate samples were directly infused at a flow rate of 3 μL/min, and anions were generated via electrospray in the negative ion mode with a heated electrospray voltage of −2.2 kV relative to ground and a transfer tube temperature of 300° C. The ion funnel RF was held at 60%. All MS survey spectra were collected at a resolving power of 500,000 at 200 m/z in the Orbitrap with a scan range of 400-3,000 m/z, an AGC target of 400,000 charges and a maximum injection time of 50 ms. To aid in desolvation, nitrogen sheath and auxiliary gas were applied at 15 and 10 arbitrary units, respectively. MS/MS experiments were briefly carried out to confirm the identity of putative adducted species. After a 30 ms NETD reaction with fluoranthene radical cations (reagent AGC target=100,000), all product ions and unreacted precursor ions were shuttled to the ion routing multipole for beam-type collisional dissociation at 25 normalized collisional energy (NEThcD) and scanned out in the Orbitrap with resolving power of 240,000 at 200 m/z. Upon fragmentation, no site-specificity was determined for adducts. All MS and MS/MS data were collected in profile mode. For increased signal-to-noise (S/N), MS spectra are the sum of 50 individual scans, with averaging performed in the vendor's post-acquisition software (XCalibur Qual Browser, version 2.2). No microscans were performed.

Gel Electrophoresis

2% agarose gels (cast with 3 μL of a 10 mg/mL aqueous stock of ethidium bromide for every 50 mL of molten gel solution, for DNA visualization) were prepared in 1×TAE buffer and run for 40 minutes at 120 V. Gel images were recorded on an Azure C400 Gel Imaging System (Azure Biosystems) using 302 nm transillumination.

Fluorescence Reaction Tracking

Fluorescence experiments were recorded on a Tecan Spark fluorescence plate reader with the following conditions: 350 nm excitation wavelength with a 5 nm bandwidth, 450 nm emission wavelength with a 10 nm emission bandwidth, 30 flashes with an integration time of 40 μs, and no delay time.

DNA Sequences

All sequences used in this paper are listed below, each named using a unique identifying code of the form dna ##. Catalyst conjugates prepared are referred to by the original sequence and the attached molecule (e.g. dna ##+dcbpy or dna ##+cTEMPO). /3AmMO/and/5AmMC6/are codes used by the DNA supplier (Integrated DNA Technologies) to refer to amino-modifications positioned at the 3′ and 5′ ends of synthetic oligonucleotides.

Sequences used in FIGS. 18-20 and 22

Sequences used in FIGS. 18-20 and 22
(SEQ ID NO: 1)
dna01 = 5′-ACAGGCTAACGAACGATCTCGAGCGC-3′
(SEQ ID NO: 2)
dna02 = 5′-GCGCTCGAGATCGTTCGTTAGCCTGT-3′
(SEQ ID NO: 3)
dna03 = 5′-CGTAGTGAGACTTACAGCTTCGTAGG-3′
(SEQ ID NO: 4)
dna04 = 5′-CCTACGAAGCTGTAAGTCTCACTACG-3′
(SEQ ID NO: 5)
dna05 = 5′-ACAGGCTAACGAACGATCTCGAGCGC-/3AmMO/-3′ d
(SEQ ID NO: 6)
na06 = 5′-/5AmMC6/-GCGCTCGAGATCGTTCGTTAGCCTGT-3′
(SEQ ID NO: 7)
dna07 = 5′-/5AmMC6/-ACAGGCTAACGAACGATCTCGAGCGC-3′
(SEQ ID NO: 8)
dna08 = 5′-CGTAGTGAGACTTACAGCTTCGTAGG-/3AmMO/-3′
Sequences used in FIG. 25
(SEQ ID NO: 6)
dna06 = 5′-/5AmMC6/-GCGCTCGAGATCGTTCGTTAGCCTGT-3′
(SEQ ID NO: 8)
dna08 = 5′-CGTAGTGAGACTTACAGCTTCGTAGG-/3AmMO/-3′
(SEQ ID NO: 9)
dna09 = 5′-ACAGGCTAACGAACGATCTCGAGCGCCCTACGAAGCTGT
AAGTCTCACTACG-3′
(SEQ ID NO: 10)
dna10 = 5′-ACAGGCTAACGAACGATCTCGAGCGCTCCTACGAAGCTG
TAAGTCTCACTACG-3′
(SEQ ID NO: 11)
dna11 = 5′-ACAGGCTAACGAACGATCTCGAGCGCTTCCTACGAAGCT
GTAAGTCTCACTACG-3′
(SEQ ID NO: 12)
dna12 = 5′-ACAGGCTAACGAACGATCTCGAGCGCTTTCCTACGAAGC
TGTAAGTCTCACTACG-3′
(SEQ ID NO: 13)
dna13 = 5′-ACAGGCTAACGAACGATCTCGAGCGCTTTTTCCTACGAA
GCTGTAAGTCTCACTACG-3′
(SEQ ID NO: 14)
dna14 = 5′-ACAGGCTAACGAACGATCTCGAGCGCTTTTTTTTTTCCT
ACGAAGCTGTAAGTCTCACTACG-3′
Sequences used in FIGS. 26-27
(SEQ ID NO: 6)
dna06 = 5′-/5AmMC6/-GCGCTCGAGATCGTTCGTTAGCCTGT-3′
(SEQ ID NO: 15)
dna15 = 5′-ACAGGCTAACGAACGATCTCGAGCGCGCGAGAAGTTAAG
ACCTATGCTCGC-/3AmMO/-3′
(SEQ ID NO: 16)
dna16 = 5′-CCTGAACCGCGAGCATAGGTCTTAACTTCTCGC-3′
(SEQ ID NO: 17)
dna17 = 5′-GCGAGAAGTTAAGACCTATGCTCGCGGTTCAGG-3′

Synthesis and Characterization

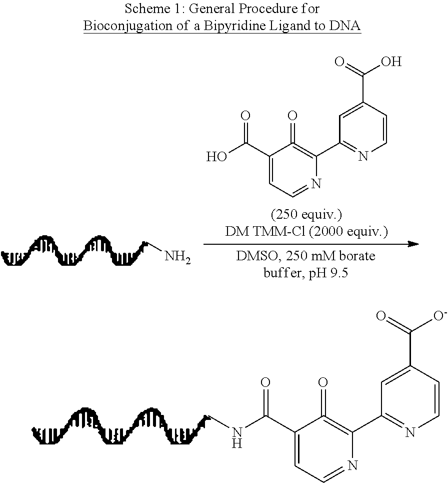

In a 1.5 mL centrifuge tube, 100 nmol of amine-modified DNA oligonucleotide (10 mM stock in nuclease-free water) was diluted with 290 μL of 250 mM sodium borate buffer, pH 9.5 and 100 μL of DMSO. 100 μL of a 0.25 M stock of 4,4′-dicarboxy-bipyridine (dcbpy, in 1:1 250 mM sodium borate buffer, pH 9.5 and DMSO, 250 equivalents), was then added, followed by 400 μL of 0.5 M DMTMM-Cl (in nuclease-free water, 2000 equivalents). The sample was vortexed thoroughly, spun down in a bench-top centrifuge, and then incubated for 16 hours at 17° C. with 500 rpm shaking in an Eppendorf thermomixer. At this point, the sample was spun down to pellet any precipitate and then concentrated by transferring the supernatant to and concentrating in a 3K Amicon Ultra-0.5 mL centrifugal filter device (prerinsed with 500 μL of nuclease-free water and spun down at 14000 rcf for 10 minutes, then loaded with sample and concentrated by spinning as before). The device was then diluted with 400 μL nuclease-free and concentrated by spinning as before, three times to remove residual ligand. The retentate was recovered and the membrane was rinsed with 50 μL nuclease-free water to maximize recovery. The concentration of crude DNA-conjugate was then checked by UV absorption at 260 nm using a NanoDrop spectrometer. The isolated conjugate was then purified by HPLC by iterative injection of 20 nmol aliquots. The product-containing fractions were then pooled, lyophilized, buffer-exchanged with nuclease-free water on a second Amicon Ultra filtration device to remove residual TEAA from the HPLC purification, and then subjected to a final lyophilization before resuspension in nuclease free water to approximately 1 mM concentration. The final concentration of conjugate was determined using a NanoDrop spectrometer. Yields ranged from 35-80 nmol of HPLC-purified DNA conjugate. To assess the integrity of oligonucleotide-catalyst conjugates, intact mass analysis via electrospray mass spectrometry (ESI-MS) was performed (see “Mass Spectrometry”).

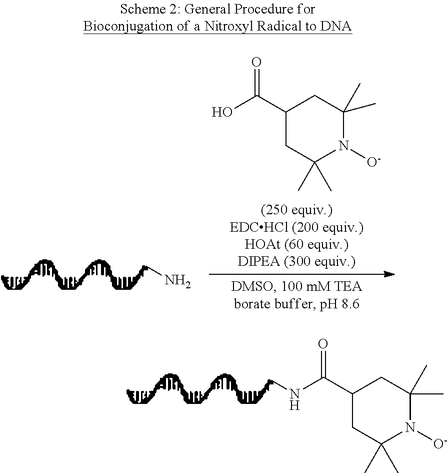

In a 1.5 mL centrifuge tube, 100 nmol of amine-modified DNA oligonucleotide (10 mM stock in nuclease-free water) was diluted with 90 μL of 100 mM triethylammonium borate buffer, pH 8.0 and 250 μL of DMSO. To this solution was added 50 μL of an 0.5 M stock of 4-carboxy-TEMPO (cTEMPO, 250 equivalents) in DMSO, 100 μL of a 0.3 M stock of EDC-HCl in DMSO (300 equivalents), 50 μL of a 0.12 M stock of HOAt in DMSO (60 equivalents) and 50 μL of a 0.6 M stock of DIPEA in DMSO (300 equivalents). The sample was then vortexed thoroughly, spun down in a benchtop microcentrifuge, and then incubated for 16 hours at room temperature without shaking. The sample was then precipitated by isopropanol precipitation (40 μL 3 M sodium acetate, 1 mL cold isopropanol, spun down at 21000 rcf at 4° C. for 30 minutes). The supernatant was decanted, and the pellet was rinsed with two 200 μL portions of cold 70% ethanol, air-dried, and resuspended to approximately 1 mM concentration with nuclease-free water. The concentration of crude DNA was then checked by UV absorption at 260 nm using a NanoDrop spectrometer. The isolated conjugate was then purified by HPLC by iterative injection of 20 nmol aliquots. The product-containing fractions were then pooled, lyophilized, and buffer-exchanged with nuclease-free water on a second Amicon Ultra filtration device to remove residual TEAA from the HPLC purification. The sample was then subjected to a final lyophilization before resuspension in nuclease free water to approximately 1 mM concentration. The final concentration of the conjugate stock was determined using a NanoDrop spectrometer. Yields ranged from 35-50 nmol of HPLC-purified DNA conjugate. To assess the integrity of oligonucleotide-catalyst conjugates, intact mass analysis via electrospray mass spectrometry (ESI-MS) was performed (see “Mass Spectrometry”).

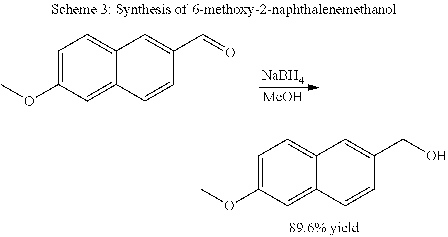

6-methoxy-2-naphthaldehyde (2 g, 10.7 mmol, 1 equivalent) was dissolved in 50 mL HPLC-grade methanol (no precautions were taken to dry the solvent) in a 100 mL round-bottom flask with stirring. Sodium borohydride (0.450 g, 11.9 mmol, 1.1 equivalents) was then added in 50 mg portions (vigorous bubbling was observed after each addition, and the color of the reaction mixture changed from clear orange to a yellow-green). 2 mL of methanol were used to rinse residual sodium borohydride from the edges of the flask into the mixture, the reaction was placed under a nitrogen atmosphere and stirred at room temperature until complete consumption of the aldehyde was observed by TLC (5% ethyl acetate in hexanes, with visualization by UV and KMnO4 staining). The solvent was then removed under reduced pressure and resulting off-white solid was redissolved in 50 mL of ethyl acetate. The organic layer was then partitioned with 20 mL of a saturated aqueous solution of ammonium chloride and 20 mL of distilled water. The aqueous layer was extracted with 3×20 mL ethyl acetate, and all organic layers were combined. The organic layers were then washed with 10 mL distilled water followed by 10 mL brine. The combined organic layers were dried over anhydrous magnesium sulfate, filtered and the solvent was removed under reduced pressure. This yielded 1.80 g (89.6% yield) of product as a beige powder. The structure was confirmed by 1H- and 13C-NMR. 1H-NMR (500 MHz, CDCl3) δ 7.76-7.70 (m, 3H), 7.45 (dd, J=8.5, 1.7 Hz, 1H), 7.18-7.11 (m, 2H), 4.82 (s, 2H), 3.92 (s, 3H), 1.72 (s, 1H). 13C NMR (126 MHz, CDCl3) δ 157.76, 136.06, 134.14, 129.37, 128.82, 127.22, 125.88, 125.59, 119.02, 105.76, 77.27, 77.02, 76.77, 65.59, 55.31.

TABLE 1
DNA Sequences¹

| SEQ ID No. | Sequence |
|---|---|
| 1 | 5′-ACAGGCTAACGAACGATCTCGAGCGC-3′ |
| 2 | 5′-GCGCTCGAGATCGTTCGTTAGCCTGT-3′ |
| 3 | 5′-CGTAGTGAGACTTACAGCTTCGTAGG-3′ |
| 4 | 5′-CCTACGAAGCTGTAAGTCTCACTACG-3′ |
| 5 | 5′-ACAGGCTAACGAACGATCTCGAGCGC- |
| | /3AmMO/-3′ |
| 6 | 5′-/5AmMC6/-GCGCTCGAGATCGTTCGTTAGCCTG |
| | T-3′ |
| 7 | 5′-/5AmMC6/-ACAGGCTAACGAACGATCTCGAGCG |
| | C-3′ |
| 8 | 5′-CGTAGTGAGACTTACAGCTTCGTAGG- |
| | /3AmMO/-3′ |
| 9 | 5′-ACAGGCTAACGAACGATCTCGAGCGCCCTACGAAGC |
| | TGTAAGTCTCACTACG-3′ |
| 10 | 5′-ACAGGCTAACGAACGATCTCGAGCGCTCCTACGAAG |
| | CTGTAAGTCTCACTACG-3′ |
| 11 | 5′-ACAGGCTAACGAACGATCTCGAGCGCTTCCTACGAA |
| | GCTGTAAGTCTCACTACG-3′ |
| 12 | 5′-ACAGGCTAACGAACGATCTCGAGCGCTTTCCTACGA |
| | AGCTGTAAGTCTCACTACG-3′ |
| 13 | 5′-ACAGGCTAACGAACGATCTCGAGCGCTTTTTCCTAC |
| | GAAGCTGTAAGTCTCACTACG-3′ |
| 14 | 5′-ACAGGCTAACGAACGATCTCGAGCGCTTTTTTTTTT |
| | CCTACGAAGCTGTAAGTCTCACTACG-3′ |
| 15 | 5′-ACAGGCTAACGAACGATCTCGAGCGCGCGAGAAGTT |
| | AAGACCTATGCTCGC-/3AmMO/-3′ |
| 16 | 5′-CCTGAACCGCGAGCATAGGTCTTAACTTCTCGC-3′ |
| 17 | 5′-GCGAGAAGTTAAGACCTATGCTCGCGGTTCAGG-3′ |

¹/3AmMO/ and /5AmMC6/ are codes used by the DNA supplier
(Integrated DNA Technologies) to refer to amino-modifications
positioned at the 3′ and 5′ ends of the oligonucleotides.

### Example 2: Switchable DNA Catalysts for Proximity Labeling at Sites of Protein-Protein Interactions

Protein-protein interactions (PPIs) and multi-protein complexes are essential for intercellular communication and for transmitting extracellular signals across the plasma membrane. Therefore, methods that determine which proteins are proximal to a specific extracellular protein of interest (POI) are important for studying cellular signaling. Traditionally, methods such as co-immunoprecipitation or photo-cross-linking have been used to determine which proteins interact with a POI. However, these methods require a direct interaction with the protein of interest, which in some cases is advantageous, but which precludes the discovery of proteins that are close to the POI without directly contacting it. Recently, an approach called proximity labeling (PL) has been developed to identify endogenous proteins within a defined radius of a POI, even if those proteins do not directly contact the POI. In PL methodologies, the POI is attached to a catalyst, which generates a reactive species that covalently tags endogenous proteins within a restricted radius, determined by the lifetime of the reactive species. Next, cells are lysed, the tagged proteins are isolated using affinity purification, and the enriched proteins are identified using mass spectrometry. Alternatively, the PL catalyst can be attached to another a non-proteinaceous biomolecule of interest or targeted to a cellular organelle.

A variety of PL catalysts have been reported, along with multiple approaches for attaching the catalyst to a POI. Some PL methods involve the attachment of an enzyme, such as the biotin ligases BioID or TurboID or a heme peroxidase, to the POI through genetic fusion or antibody targeting. In the case of biotin ligases, the enzyme converts endogenous biotin to an adenylate ester, which reacts with nucleophilic residues on proximal endogenous proteins. Heme peroxidases, such as horseradish peroxidase (HRP) and enhanced ascorbate peroxidase 2 (APEX2), catalyze the H₂O₂-dependent oxidation of biotin phenol into a phenoxy radical, which forms covalent adducts primarily with surface-exposed tyrosine residues.

As an alternative to enzymes, a variety of synthetic photocatalysts have recently been developed for PL. These photocatalysts are targeted to the POI using an antibody or other binding moiety, which is advantageous for use in untransfected cells, and the requirement for photo-irradiation allows for precise control over reaction time. Furthermore, several synthetic photocatalysts have expanded the scope of PL labeling mechanisms beyond those accessible with genetically encodable enzyme catalysts. Ruthenium, iridium, or flavin catalysts have been used for the oxidative coupling of phenols, anilines, or urazoles onto tyrosine or tryptophan residues of endogenous proteins. In a mechanistically distinct approach, photosensitizers such as dibromo-fluorescein, thiorhodamine, and BODIPY were used to generate singlet oxygen in the presence of nucleophilic probes, leading to covalent modification of histidine residues. A genetically encoded photosensitizer (miniSOG) was recently reported to catalyze intracellular PL by the same mechanism. An iridium photocatalyst or a variety of organic dyes were used to convert aryl azides into electrophilic moieties that labeled nucleophilic protein residues, while a Sn^{IV} chlorin e6 photocatalyst or osmium photocatalyst was used to convert aryl azides to aminyl radicals or triplet nitrenes. Using an iridium photocatalyst, it was shown that a diazirine could be converted to a carbene with a 2-nanosecond lifetime, which is advantageous for achieving a short labeling radius.

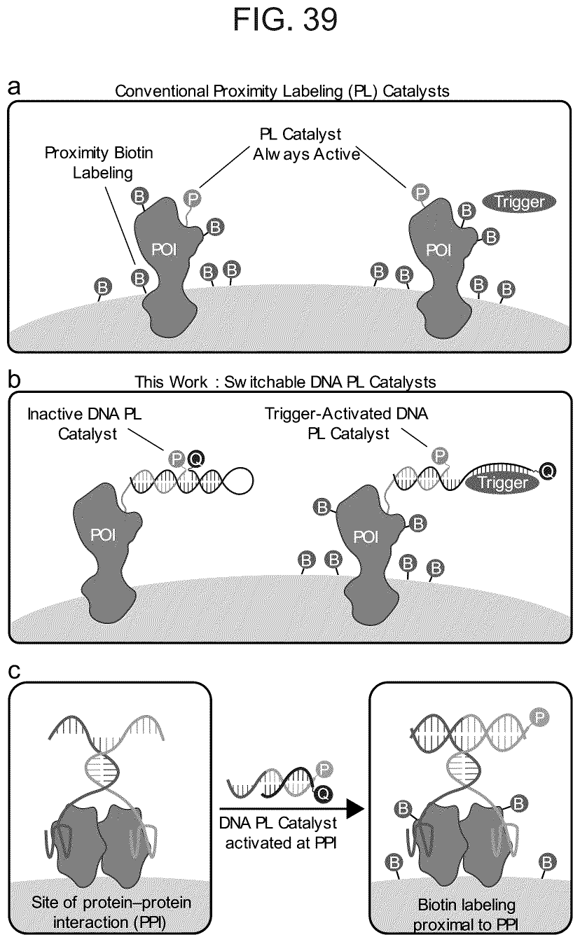

While PL methods have been transformative in elucidating proteomes proximal to a POI, new PL methods are needed for determining proteomes in subcellular regions that cannot be defined by the location of a single POI (FIG. 39). For example, dimerized cell surface receptors are of interest because their dysregulation contributes to abnormal cell proliferation, cell survival, and invasion of many cancers. Elucidating proteomes proximal to these PPIs could enable discovery of proteins that are selectively recruited to the dimerized receptors, but not their monomeric counterparts. Split enzyme systems are one approach to address this challenge. Split enzymes, which require that two interacting partners bind together to reconstitute the active enzyme, have been reported for split BioID or split TurboID, split APEX, and split HRP. However, split enzyme PL systems are typically used as genetic fusions of the split protein fragments to the two PPI partners, which can perturb the proteins of interest or disrupt their trafficking to the cell surface.

Here, inventors report DNA-based switchable PL catalysts that can be attached to a POI and become active only when a secondary molecular trigger is present (FIG. 39). These switchable catalysts consist of three components: 1) a DNA oligomer that changes conformation in response to a molecular trigger, 2) a PL photocatalyst, and 3) a spectral quencher, which renders the photocatalyst inactive when the two are in proximity. By default, the DNA catalyst adopts a conformation in which the photocatalyst is inactive due to proximity with the quencher, but the DNA changes conformation in response to a specific molecular trigger, altering the distance between photocatalyst and quencher and activating photocatalytic activity. Because many conformation-switching DNA aptamers have been reported previously, the molecular trigger can be a protein, small molecule, or metal ion. Inventors show that biotinylation of proteins in vitro and on a solid support can be activated by a single stranded DNA trigger, and inventors demonstrate that the photocatalyst can be activated at the site of a cancer-relevant PPI—the c-Met homodimer—on living mammalian cells (FIG. 39). In this study, inventors use a heteroleptic polypyridyl Ru(II) complex as a photocatalyst for the oxidative coupling of biotin phenol to tyrosine residues, but in principle, any of the recently reported PL photocatalysts can be incorporated into this system, as long as a suitable spectral quencher can be identified.

As an initial photocatalyst to demonstrate DNA-based switchable PL catalysis, inventors selected a heteroleptic Ru(bpy)₂(phenanthroline) complex for oxidation of the small molecule biotin phenol (BP) because Ru(bpy)₃-type complexes are well-established for oxidative phenol couplings on proteins. Ru(bpy)₃ has also been shown to catalyze the oxidative coupling of two BP molecules in the presence of ammonium persulfate (APS), as well as the oxidative coupling of BP to tyrosine residues. Additionally, these complexes have a catalytically active photoexcited state that can be quenched through proximity to a spectral quencher, and they are DNA compatible.

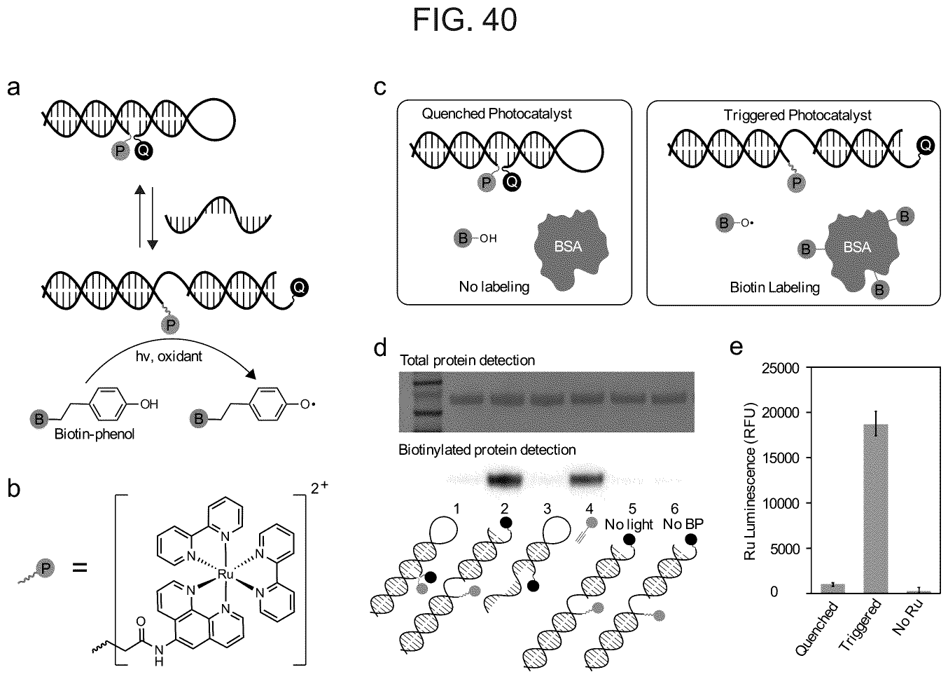

Inventors synthesized the switchable DNA catalyst by appending abiotic groups onto individual single-stranded DNA (ssDNA) strands, then annealing the strands together (FIG. 40). The quencher oligo incorporates a loop-hairpin structure, and the photocatalyst oligo anneals to the hair-pin-containing strand, placing the photocatalyst immediate-ly proximal to the quencher. The DNA sequences were de-signed following established principles of fluorescence molecular beacons, such that addition of a trigger ssDNA oligo alters the conformation of the DNA scaffold, thus in-creasing the distance between the photocatalyst and quencher and turning on photocatalytic activity. Inventors used a proprietary quencher called “RQ” (Integrated DNA Technologies) because it has strong spectral overlap with the excitation spectrum of the Ru complex. To create the Ru-DNA conjugate, inventors first synthesized a Ru(bpy)2(phenanthroline-alkyne) complex (Scheme 8, 9). Next, inventors installed the Ru complex onto a ssDNA oligo bearing an azide modification using copper-catalyzed azide-alkyne coupling, followed by HPLC purification (Scheme 10). Inventors confirmed the identity of the conjugated oligo using gel electrophoresis, HPLC, and mass spectrometry.

Inventors assembled the switchable DNA PL catalyst by combining the quencher and Ru ssDNA oligos with a slight (1.4 eq) excess of quencher DNA to ensure full quenching.

Next, inventors investigated whether the DNA catalyst exhibited PL activity that could be triggered by the presence of a ssDNA trigger oligo, which is complementary to the loop region of the hairpin sequence. Inventors first used Ru luminescence to assess whether the Ru complex was quenched in the “off” conformation and whether luminescence could be activated in the “on” state. Inventors observed a 19-fold increase in luminescence upon addition of the trigger strand.

Inventors next investigated the ability of the switchable DNA PL catalyst to label proteins in vitro. Inventors combined the DNA catalyst with a purified protein (bovine serum albumin, BSA), in the presence or absence of its DNA trigger, along with BP and the oxidant ammonium persulfate (APS). Inventors included APS as a terminal oxidant, following the precedent that APS can oxide the excited state *Ru(II) com-plex to a Ru(III) complex which can oxidize BP to afford the phenoxy radical and ground state Ru(II) complex. The DNA catalyst was not bound to BSA in this experiment, but the DNA catalyst and BSA were added at high concentrations such that BSA could be tagged by biotin phenoxy radicals generated by the catalyst. Photocatalytic BP oxidation was initiated by irradiation with blue light (450 nm), and Western blotting was used to detect covalent biotin modifications of BSA. In its “off” state, the DNA catalyst exhibited similar protein labeling to negative controls in which BP was omitted (FIG. 40). Upon addition of the ssDNA trigger, a significant increase in biotin labeling was observed. BSA labeling was abolished when light or BP was omitted. Ru luminescence correlated well with photo-catalytic activity (FIG. 40E). Inventors confirmed that the DNA is not significantly biotinylated during the labeling procedure, consistent with a previous finding that biotin phenoxy radicals exhibit very low reactivity with DNA.

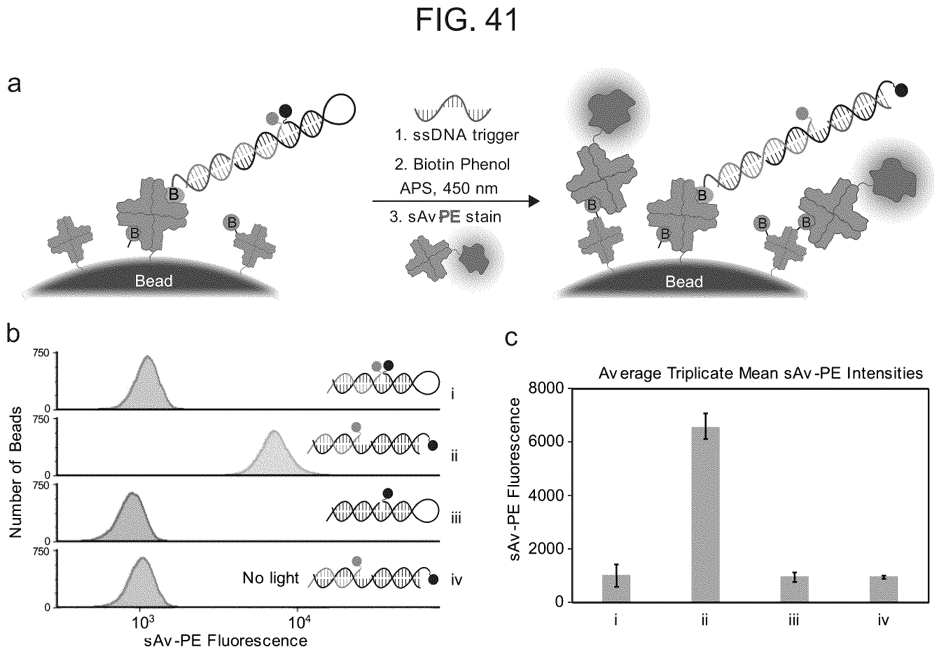

Next, inventors tested whether the switchable DNA catalyst could become activated on streptavidin-coated magnetic beads. To target the DNA catalyst to streptavidin on the beads, inventors attached a ssDNA overhang and annealed it to a complementary biotinylated ssDNA oligo (FIG. 41A). Inventors next added a ssDNA oligo trigger to switch the DNA catalyst into its active conformation, performed photocatalytic BP labeling, and stained with a fluorescent streptavidin-phycoerythrin conjugate (sAv-PE). Flow cytometry showed bright fluorescence that was specific to beads with activated DNA catalysts (FIG. 41B). Inventors observed a 6.4-fold in-crease in average bead fluorescence when the ssDNA trig-ger was added (FIG. 41C). This fluorescence increase was not observed when the ssDNA oligo trigger, BP, Ru, or light during photocatalysis was omitted.

After observing DNA catalyst activation with a ssDNA trigger, inventors next explored proximity driven activation using a split invader system, toward the goal of activating PL at the sites of PPIs. For this activation mechanism, a photocatalyst ssDNA strand and a quencher ssDNA strand are annealed in a duplex. In the duplex conformation, the photocatalyst is inactive due to its proximity to the quencher. Two “split invader” oligos are introduced, with each split invader being attached to a DNA aptamer that recruits it to a specific epitope. If the two split invaders bind in immediate proximity on the surface of a bead or a living cell, they anneal to each other to form a “full invader” that anneals to the photocatalyst-containing duplex and displaces the quencher strand, thus activating PL. Importantly, both split invader oligos are necessary to activate PL catalysis.

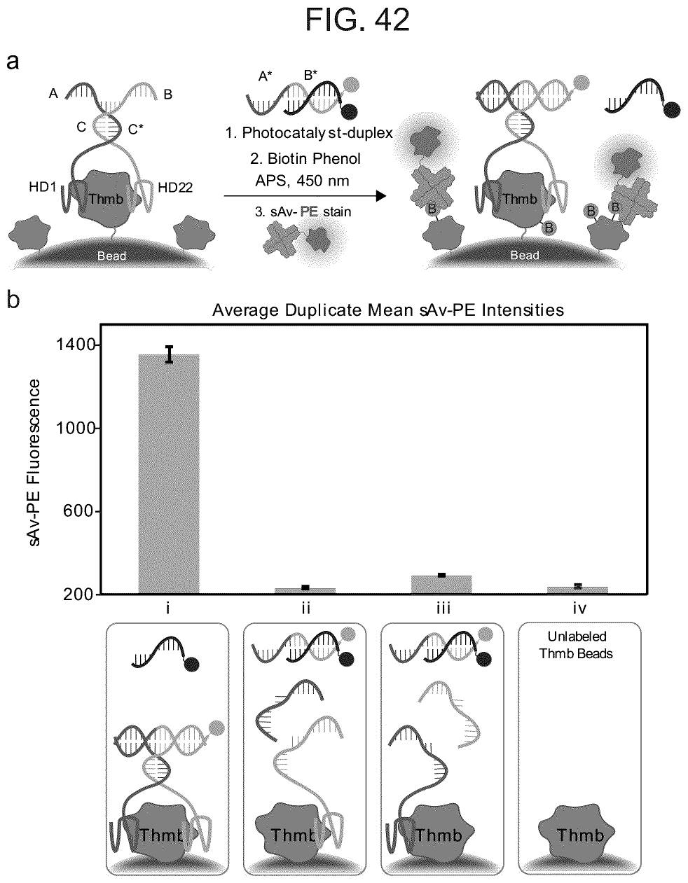

For initial validation of the split invader system, inventors used thrombin coated beads and appended the split invaders to two thrombin aptamers, HD1 and HD22, which bind to distinct sites on thrombin. Each aptamer sequence was extended to contain a short ssDNA sequence (C or C*) complementary to the other DNA construct (FIG. 42A). Inventors chose a length of 4 base pairs for the C—C* motif to ensure the duplex forms only when the oligos are bound in proximity on the same molecule of thrombin. After attaching thrombin to carboxylic acid-functionalized magnetic beads, inventors introduced both aptamer-split invader oligos and the photocatalyst-quencher duplex. The beads were subjected to BP labeling followed by sAv-PE staining. PE signal was observed only when both split invaders were appended to the appropriate aptamers (HD1-CA+HD22-C*B) as seen in FIG. 42B. If one of the split invaders lacked the appropriate aptamer sequence (CA, or C*B), no turn-on was observed, indicating the two split invaders need to be proximal to activate PL catalysis. Additionally, no PE signal was observed when Ru-DNA, BP, or light was omitted.

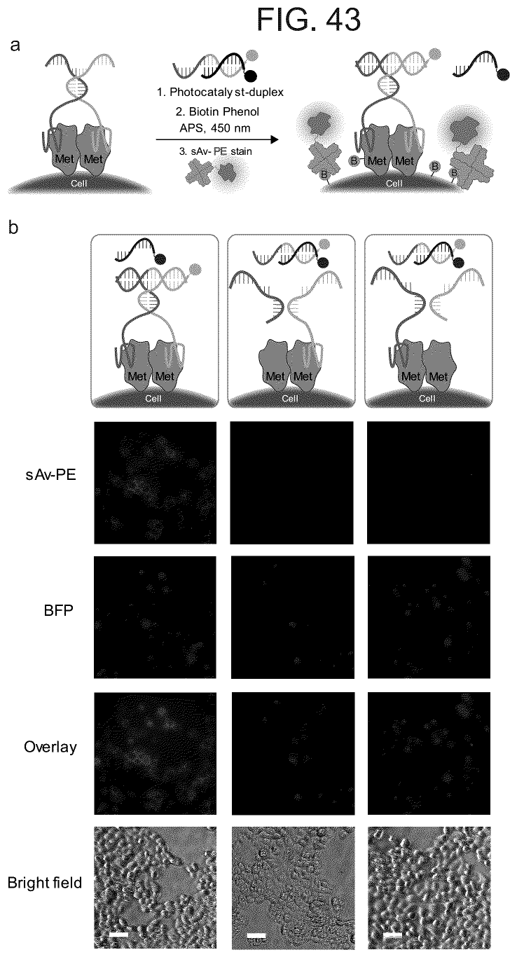

Inventors applied the split invader approach to activate PL at the sites of PPIs on living mammalian cells. Inventors selected homodimerization of c-Met, a receptor tyrosine kinase that is expressed on epithelial cells, as the target PPI. While C-Met plays a crucial role in several biological processes such as wound healing, dysregulation of the receptor or its natural ligand, hepatocyte growth factor (HGF), can lead to invasive growth of aggressive tumors. To activate PL selectively at c-Met homodimers, inventors employed a split invader approach that was previously used for fluorescence detection of c-Met homodimerization. In this design, each split invader construct consists of the c-Met aptamer elongated on either the 5′ or 3′ end with a poly-T linker, an 8-nucleotide complementary region (C or C*), and one of the split invader sequences (A or B) (FIG. 43A). While the size of the DNA scaffold means that the photocatalyst is projected several nanometers away from c-Met, potentially increasing the labeling radius, this distance is shorter than the >50 nm sweep radius that has been reported for antibody constructs commonly used in photocatalytic PL.

Inventors transfected HEK293T cells with a plasmid encoding C-Met, along with a plasmid encoding blue fluorescent protein localized to the nucleus (BFP-NLS) as a co-transfection marker. Inventors treated the transfected cells with the split invader oligos and the photocatalyst-quencher duplex. After oligo labeling, cells were photoirradiated in the presence of BP, then stained with sAv-PE. By epi-fluorescence microscopy, inventors observed strong PE signal on the perimeter of transfected cells when both intact split invader-aptamers were added (Apt-CA+Apt-C*B), as shown in FIG. 43B. When either invader had the aptamer sequence omitted (CA or C*B), no PE signal was detected, indicating that both split invaders need to be bound c-Met to activate the photocatalyst. Controls with no invader strands added, no BP, or no light also showed no signal.

Inventors have developed a new approach for activation of PL photocatalysis in highly specific subcellular locations. The approach offers multiple advantages. First, it takes advantage of the established ability of DNA conformational changes to alter the distance between a chromophore and a spectral quencher, as in molecular beacon probes. DNA oligomers have been reported to bind a wide array of chemical stimuli including small molecules, ions, and proteins. Thus, switchable DNA PL catalysts can in principle be tailored to respond to any of these stimuli. Furthermore, split invader DNA strands allow for activation of PL at PPIs, as inventors demonstrate in this study. Because this method uses a photocatalyst, PL is controlled by light, which means that labeling is dependent on three conditions: 1) the presence of the DNA catalyst, 2) the presence of the molecular trigger, and 3) photo-irradiation. The switchable DNA PL catalyst platform is compatible with any photocatalyst for which a suitable spectral quencher exists, thus expanding the possible catalyst and substrate combinations to the large toolkit of photoredox chemistry.

TABLE 2
Other targets of the disclosed technology, including aptamers, protein-
protein interaction (PPI) protein pairs, and pairs of antigens that are
overexpressed in certain types of cancer. These pairs of antigens over
expressed in cancers don't necessarily interact with each other, but
inventors propose that the split DNA invader probes can also be activated
on the surface of cancer cells overexpressing two antigens of interest, even
if they are not directly interacting in a PPI.

| | | | Aptamer/antibody (specific ones |
|---|---|---|---|
| | | | listed, although any |
| | | Protein | aptamer/antibody/ligand could be |
| Cancer | Protein | partner | used) |
| Breast | HER2 | HER3, | HER2 aptamer: HB5 aptamer |
| Cancer | | HER4, | HER3 aptamer: A30 aptamer |
| (all PPIs) | | EGFR, | HER4 anitbody: H4.77.16 |
| | | HER2 | EGFR aptamer: E07 aptamer |
| Many | c-MET | c-MET | c-MET aptamer: CLN0003 aptamer |
| different | | | |
| cancers | | | |
| Glioblastoma | HER2 | IL 13R- | HER2: FRP5 (antibody) |
| | | alpha-2 | IL13R-alpha-2: 1L13 mutein |
| | | | (protein) |
| Ovarian | PDL1 | MUC16 | PDL1 aptamer: XQ-P3 aptamer |
| Cancer | | | MUC16 aptamer: MAB56091 |
| | | | antibody |
| Leukemia | CD19 | CD22 | antibodies |
| B-cell | | | |
| Lung Cancer | EGFR | c-MET | antibodies |
| B-cell | CD47 | CD19 | antibodies |
| lymphoma | | | |
| Non-hodgkin | CD20 | CD47 | antibodies |
| lymphoma | | | |
| Leukemia | HLA- | CD45 | antibodies |
| | A2 | | |

### Materials

All solvents used in this study were purchased from Sigma Aldrich in HPLC Grade and used without further purification. All DNA oligos were purchased from Integrated DNA Technologies with standard desalting or HPLC purification if required. Vendors for chemicals are listed within the specific procedures. Plasmid for c-Met (HG10692-CF) was purchased from Sino Biological. Nuclear-localized BFP was a previously reported plasmid, LAP-BFP-NLS.¹ HPLC analysis and purification of DNA conjugates

Reverse-phase HPLC analysis and purification of DNA conjugates were performed on a Shimadzu Prominence HPLC system with analysis at 254 nm unless stated otherwise. HPLC analysis and purification were performed using a 4.0×300 mm Kromasil 100-5-C18 column with a 1.0 mL/min flow under ion-pairing conditions using aqueous 100 mM triethylammonium acetate pH 7.0 (TEAA) as mobile phase A and acetonitrile as mobile phase B (using a gradient from 10-20% B over 10.5 minutes, followed by a half-minute ramp to 80% and a half-minute wash at 80% B and then re-equilibration at 10% B), with a column oven temperature of 50° C.

### NMR Spectroscopy

¹H and ¹³C{¹H} NMR spectra were recorded on a Bruker Avance 500 MHz spectrometer. Chemical shifts (δ) are given in parts per million and referenced to TMS (if CDCl₃ was used as the solvent).

### UV-Vis Spectroscopy

DNA concentrations were determined by UV-Vis spectroscopy (absorption at 260 nm and/or 450 nm) using a Nanodrop One Spectrophotometer (ThermoFisher Scientific).

### Mass Spectrometry

ESI-MS Small molecule high resolution mass spectrometry spectra were obtained on a Thermo Q Exactive™ Plus by the mass spectrometry facility at the University of Wisconsin-Madison. The purchase of the Thermo Q Exactive™ Plus in 2015 was funded by NIH Award 1S10 OD020022-1 to the Department of Chemistry. ESI-MS of Ru-functionalized DNA oligos were obtained on a Thermo Q Exactive™ Focus within the mass spectrometry facility at the University of Wisconsin-Madison

### Luminescence Measurements

Luminescence Measurements were recorded on a Tecan Spark fluorescence plate reader with the following conditions: 450 nm excitation wavelength with a 10 nm bandwidth, 620 nm emission wavelength with a 10 nm emission bandwidth, 30 flashes with an integration time of 40 μs, and no delay time.

### Fluorescent Microscopy Imaging

Imaging was performed with an Eclipse Ts2 Inverted Routine Microscope with a 20× objective. PE (550/50 nm LED excitation, 630/75 nm emission), BFP (390/38 nm excitation, 475/90 nm emission), and differential interference contrast (DIC) images were collected using a Panda USB3.1 sCMOS Camera (PCO). Images were analyzed using NIS-Elements L and fluorophore channels in each experiment were normalized to the same intensity ranges. Acquisition times ranged from 100 ms (BFP) to 3 s (PE).

### In Vitro BSA Labeling

In a 600 μL PCR tube, a 22.5 μL solution was prepared consisting of 1 μM Ru-DNA, 1.4 μM Quencher DNA, and 2.6 μM Trigger DNA in phosphate buffered saline with Mg 2+(PBS-Mg: 137 mM NaCl, 2.7 mM KCl, 10 mM Na₂HPO₄, 1.8 mM KH₂PO₄, 10 mM MgCl₂). The solution was incubated at room temperature for 30 minutes to allow DNA to hybridize. A 2× master mix labeling solution was then made containing 80 μM Bovine Serum Albumin (BSA, MP Biomedicals), 200 μM Biotin Phenol (Sigma), and 2 mM ammonium persulfate (VWR) in 10 mM 2-(N-morpholino)ethanesulfonic acid (MES, Fisher Scientific) buffer, pH 7.4, supplemented with 10 mM MgCl₂. After the DNA incubation period, 22.5 μL of master mix was added to samples. The samples were then vortexed and briefly centrifuged. Samples were illuminated on their side while shaking with light set to 50% intensity for 5 minutes. Samples were quenched with 2 μL of 210 mM dithiothreitol (DTT, Goldbio) and allowed to sit for 5 minutes. For western blot analysis, 1.5 μL of crude sample was combined with 3.5 μL of PBS buffer, pH 7.4 and 2 μL of SDS loading dye (0.25 M Tris, pH 6.8, 10% w/v SDS, 30% v/v glycerol, 10 uM DTT, 450 uM bromophenol blue). Samples were then denatured at 95° C. for 10 minutes. The samples were loaded onto an 8% Bolt Bis-Tris SDS page gel (ThermoFisher) and run at 200 V for 8 minutes with MES-SDS running buffer (50 mM MES, 50 mM Tris Base, 0.1% SDS, 1 mM EDTA, pH 7.3). Protein was transferred to a nitrocellulose membrane for 1 hour at 10 V. Total protein was visualized by Ponceau S staining ((0.1% w/v Ponceau S (EMD Millipore) in 5% acetic acid/water, 5 minutes) followed by successive rinses with DI water to reveal protein bands. The membrane was soaked in water until the bands disappeared, and the membrane was then blocked with 3% BSA in 1.2×TBS-T (Tris Buffered Saline with Tween-20 (DOT Scientific), pH 7.5 containing 24 mM Tris, 300 mM NaCl, v/v) overnight. The membrane was then stained with 1:5000 SA-HRP (ThermoFisher) in 1% BSA, 1.2×TBS-T for 15 minutes. The membrane was washed 4×3 minutes with 1.2×TBS-T. The membrane was treated with a solution containing equal volumes of Radiance Q substrate (Azure Biosystems) and Radiance Peroxide (Azure Biosystems) then immediately visualized with an Azure C400 Gel Imaging System (Azure Biosystems) using the Chemi setting.

### Test for DNA Biotinylation

In a 0.6 mL PCR tube, DNA (either unmodified, Ru-modified) and Ru-alkyne were combined to a final concentration of 2 μM in PBS with 10 mM each KCl and MgCl₂. Then, an equal volume of labeling solution containing 200 μM BP (or no BP) and 2 mM ammonium persulfate (APS) in 10 mM MES buffer (pH 7.4 with 10 mM MgCl₂) was added. Samples were briefly centrifuged in a tabletop centrifuge. The tubes were either kept in the dark or irradiated for minutes with the photoreactor at 50% power, while laying on their sides with lids closed. After irradiation, 2 μL of 210 mM DTT was added to quench the reaction. Biotin-DNA was purchased from IDT with a single biotin modification per DNA molecule and was not subjected to the photocatalytic reaction conditions, but it was subjected to the same workup procedures as the other samples. Amicon Ultra 3K MWCO filter devices were used to remove excess BP and buffer exchanged into PBS. Three 1 μL dots were spotted in triplicate on a nitrocellulose membrane. DNA was crosslinked to the nitrocellulose membrane by UV illumination for 5 minutes. The nitrocellulose membrane was then blocked for 30 minutes with 2% BSA in 1.2×TBS-T (Tris Buffered Saline with Tween-20, pH 7.5 containing 24 mM Tris, 300 mM NaCl, 0.6% v/v) followed by staining with 1:2000 SA-HRP in 1% BSA in TBS-T for 1 hour. The membrane was washed 4×3 minutes with 1.2×TBS-T. Biotin was visualized as above. Using FIJI image analyzer, each sample's row was converted to a curve corresponding to the intensity of the three dots in each lane, with each dot producing one peak in the curve. For one row, all three peaks were integrated under the curve to quantify the luminescence produced by each of the three dots. The average intensity was calculated for each of the three replicates.

### Streptavidin-Coated Bead Labeling

For each sample, 2 μL of Dynabeads™ M-270 Streptavidin coated beads (ThermoFisher) were placed into 1.5 mL centrifuge tubes, then washed twice with 50 μL binding buffer (50 mM Tris, pH 7.4, 300 mM NaCl, 20 mM KCl, 10 mM MgCl₂, 1 mM EDTA, 0.01% tween) containing 1% BSA, using a magnetic rack to separate the magnetic beads during washes. 20 μL of probe solution, containing 1 μM of each DNA oligo, was then added to the beads, briefly vortexed and centrifuged, then incubated on their sides using a vortex with adapter head for 30 minutes at room temperature. Two washes were then performed with 50 μL of wash buffer (50 mM Tris, pH 7.4, 300 mM NaCl, 20 mM KCl, 10 mM MgCl₂). Next, 20 μL of 20 μM trigger DNA oligo in wash buffer was added or wash buffer containing no DNA. After a brief vortex and centrifuge, the samples were incubated using vortex again for 30 minutes at room temperature. Samples were then washed one time with 50 μL MES binding buffer (10 mM MES, pH 7.4 with 0.5 mM EDTA, 100 mM NaCl, 20 mM KCl, 10 mM MgCl₂). Beads were then suspended in 100 μL cold BP labeling solution (100 μM BP, 1 mM APS, MES binding buffer, 10% DMSO), briefly vortexed and centrifuged then exposed to irradiation for 5 minutes while on ice on a vortex head, using the photoreactor as described above and shown in Irradiation Setup. After irradiation, samples were washed two times with 50 μL wash buffer, then incubated with 1:100 sAv-PE in binding buffer containing 0.1% BSA for 40 minutes at room temperature on vortex head. Samples were then washed two times with 50 μL of wash buffer. For flow cytometry analysis, samples were diluted to a total volume of 500 μL of wash buffer, and data was collected on a Fisher Attune analysis cytometer. Samples from tubes were processed at 100 μL/min flow rate, 20,000 gated events per sample (see Figure S9 for gating only single beads), using a 561 nm laser with 577 long pass filter for excitation and a 585/16 emission filter.

### Thrombin-Coated Bead Preparation

Human α-Thrombin was covalently linked to Invitrogen Dynabeads MyOne Carboxylic Acid beads according to the manufacturer's instructions. Briefly, 50 μL, (0.5 mg) of bead suspension was washed two times with 50 μL, 10 mM MES buffer, pH 6. Beads were then incubated with 200 μL, of chilled 10 mg/mL EDC solution in MES buffer for 30 minutes on ice, while shaking on a vortex head. EDC solution was removed, beads were washed one time with 50 μL, MES buffer, then suspended in 25 μg of thrombin in 200 μL, MES buffer. Protein solution and beads were incubated for 3 hours on ice while shaking on a vortex head. Protein labeling solution was removed, and beads were washed three times with 1 mL PBS supplemented with 0.1% Tween-for 10 minutes at room temperature shaking on a vortex head. Beads were stored at 4° C. at a concentration of 5 mg/mL in 100 μL PBS supplemented with 0.1% Tween-20 and 0.1% BSA for up to one month.

### Thrombin-Coated Bead Labeling

For split invader labeling, each sample contained 2 μL (10 μg) of thrombin-coated magnetic bead suspension. Beads were washed twice with 50 μL binding buffer (50 mM Tris, pH 7.4, 300 mM NaCl, 20 mM KCl, 10 mM MgCl₂, 1 mM EDTA, 0.01% tween) supplemented with (w/v) BSA. Labeling solutions were prepared containing 1 μM split invader DNA, 1 μM Ru-DNA, 1.5 μM Quencher DNA (Ru-DNA and Quencher DNA were pre-annealed at final concentration of 5 μM prior to addition to labeling solution) in 20 μL of binding buffer. After initial washes, beads were incubated with labeling solutions for 30 minutes at room temperature while shaking on a vortex head. One 50 μL wash was performed using binding buffer, and one 50 μL wash was performed using labeling buffer (10 mM MES, pH 7.4, 1 mM EDTA, 300 mM NaCl, 20 mM KCl, 10 mM MgCl₂). BP labeling solution was prepared with 100 μM BP, 1 mM APS, 10% DMSO in labeling buffer. Beads were suspended in 100 μL BP labeling solution, briefly vortexed and centrifuged, then exposed to irradiation for 5 minutes while on ice on a shaking vortex head, using the photoreactor as described above and shown in Irradiation Setup. After irradiation, samples were washed two times with 50 μL binding buffer, then incubated with 1:100 sAv-PE in binding buffer containing 0.1% BSA for 40 minutes at room temperature on vortex head. Samples were then washed two times with 50 μL of wash buffer. For flow cytometry analysis, samples were diluted to a total volume of 500 μL of wash buffer, and data was collected on a Fisher Attune analysis cytometer; 100,000 gated events per sample, using a 561 nm laser with 577 long pass filter for excitation and a 585/16 emission filter. Events shown in the histograms and used for calculations in bar graphs were gated for single beads (excluding multiple beads bound together); see S9 for gating strategy.

### Mammalian Cell Culture

HEK 293T cells from ATCC (CRL-3216) were cultured as a monolayer in DMEM (ATCC) supplemented with 10% (v/v) FBS (Corning) and 2 mM L-glutamine (ATCC) at 37° C. under 5% CO₂. For fluorescence imaging experiments, cells were grown in TC-treated 48-well plates (Greiner Bio-One). Plates were pre-treated with 13 μg/mL human fibronectin (Sigma) for one hour at room temperature to improve cell adherence. Plates were washed once with Dulbecco's phosphate-buffered saline (DPBS, Sigma), pH 7.4 with MgCl₂ and CaCl₂ prior to plating cells. Cells were transfected at 30-50% confluence with 1.3 μL Lipofectamine2000 (Invitrogen) using 500 ng c-Met plasmid (HG10692-CF) and 100 ng BFP-NLS plasmid per well in serum-free media for 2 hours, after which the media containing Lipofectamine was replaced with fresh serum-containing media. The cells were then placed back in the 37° C., 5% CO₂ incubator for 12-18 hours before cell labeling.

### Live Cell Immunostaining to Validate Protein Expression

BFP and c-Met transfected cells were removed from the incubator 12-18 hours after transfection and allowed to equilibrate to room temperature. Cells were washed once with DPBS at room temperature. 200 μL of non-conjugated primary antibody (MABS1916, Sigma. 1:500 in 1% BSA-DPBS) solution was added to each well of a 48-well plate. Antibodies were incubated with cells on ice for 20 minutes followed by two washes with ice-cold DPBS. 200 μL of secondary antibody (A11004, Life Technologies. 1:500, in 1% BSA-DPBS) was then added. Secondary labeling solutions were incubated with cells on ice for 20 minutes followed by two washes with ice-cold DPBS, after which the cells were imaged.

### DNA Probe Preparation for Live Cell Labeling

DNA probes for live cell labeling were prepared by combining equimolar split invaders with Ru-Duplex (1:1.5 Ru-DNA: RQ-DNA) at a final concentration of 300 nM split invaders and Ru-DNA in 0.1 mg/mL Herring Sperm DNA (Sigma) DPBS supplemented with 10 mM MgCl₂.

### Live Cell Labeling Using DNA Probes

After transfection (12-18 hours) media was replaced with 100 ng/mL Hepatocyte Growth Factor (HGF, PeproTech) in serum containing media. After 30 minutes in the incubator, cells were removed and allowed to equilibrate to room temperature. Cells were washed once with DPBS at room temperature. 150 μL of DNA probe solution was added to each well of a 48-well plate. Probes were incubated with cells at room temperature for 45 minutes followed by two washes with DPBS supplemented with 10 mM MgCl₂, the first wash at room temperature, the second on ice.

### Live Cell BP Labeling

Following labeling with DNA probes, a BP labeling solution was prepared containing 100 μM biotin phenol and 1 mM APS in cold 10 mM MES buffer, pH 7.4 with 10 mM MgCl₂ and 10% DMSO. Labeling solution was added to cells (200 μL for 48-well plate) immediately prior to irradiation on ice for 5 minutes using photoreactor set to 50% power. After irradiation, cells were washed twice with DPBS. For fluorescence microscopy, cells were fixed with 4% formaldehyde in DPBS for 15 minutes on ice, washed thrice with cold DPBS and blocked for 10 minutes with 1% BSA in DPBS on ice. Cells were then stained with 1:500 sAv-PE (Invitrogen) in 1% BSA-DPBS for 20 minutes on ice. Cells were washed three times with DPBS prior to imaging (see Fluorescent Microscopy Imaging).

### DNA Sequences

All sequences of DNA used in this paper are shown below, each named with a code of the form dna ##. Ruthenium conjugates are referred to by the original sequence and the attached Ru molecule (e.g. Ru-dna ##). /5AzideN/ and /3IAbRQSp/are codes used by the DNA supplier (Integrated DNA Technologies) to refer to azide and RQ quencher modifications on the 5′ and 3′ ends of synthetic oligonucleotides, respectively. /5Biosg/ and /3Bio/are codes used by IDT to refer to biotin modifications on the 5′ and 3′ ends of synthetic oligonucleotides, respectively.

Sequences used in FIG. 40

Sequences used in FIG. 40
(SEQ ID NO: 18)
dna01 = 5′-TTGGGTTCACTGCACCTGGATCGAGCCTTTACACCGAC
TCCAACAGCTCG-/3IAbRQSp/-3′
(SEQ ID NO: 19)
dna02 = 5′-/5AzideN/-TCCAGGTGCAGTGAACCCAA-3′
(SEQ ID NO: 20)
dna03 = 5′-CTGTTGGAGTCGGTGTAAAGG-3′
(SEQ ID NO: 21)
dna04 = 5′-TCATCGAGCCACAAG-3′
(SEQ ID NO: 35)
dna05 = 5′-TTGGGTTCACTGCACCTGGATCGAGCCTTTACACCGAC
TCCAACAGCTCG-3′
(SEQ ID NO: 22)
dna06 = 5′-GTAGTATATATCTTGCAGCGTTGGCGAAGTTAAGACCT
ATGCGCCA/3IAbRQSp/-3′
(SEQ ID NO: 36)
dna13 = 5′-TCCAGGTGCAGTGAACCCAA-3′
Sequences used in FIGS. 41
(SEQ ID NO: 23)
dna07 = 5′-/5AzideN/-CGCTGCAAGATATATACTACTCGCTAAA
CTAAAGATTGGA-3′
(SEQ ID NO: 24)
dna08 = 5′-/5Biosg/-TTTTTTTTTTTTTTTCCAATCTTTAGTTT
AGCG-3′
(SEQ ID NO: 43)
dna09 = 5′-CGCATAGGTCTTAACTTCGCCAA-3′
Sequences used in FIGS. 42
(SEQ ID NO: 25)
dna10 = 5′-ACT GCA CCT GGA /3IAbRQSp/-3′
(SEQ ID NO: 26)
dna11 = 5′-GGT TGG TGT GGT TGG TTT TTT TTT GTG
CCT GCA CCT GGA-3′
(SEQ ID NO: 27)
dna12 = 5′-GGG TTC ATT GCA CTT TTT TTT TTT TTT
TAG TCC GTG GTA GGG CAG GTT GGG GTG ACT-3′
(SEQ ID NO: 28)
dna13 = 5′-GTG CCT GCA CCT GGA-3′
(SEQ ID NO: 29)
dna14 = 5′-GGG TTC ATT GCA CTT-3′
Sequences used in FIGS. 43
(SEQ ID NO: 30)
dna15 = 5′-TGG ATG GTA GCT CGG TCG GGG TGG GTG
GGT TGG CAA GTC T TTT TTT TTT TTT TTT CA GTG AGG
CT GCA CCT GGA-3′
(SEQ ID NO: 31)
dna16 = 5′-TT GGG TTC A TT CCT CAC TG TTT TTT
TTT TTT TTT TGG ATG GTA GCT CGG TCG GGG TGG GTG
GGT TGG CAA GTC T-3′
(SEQ ID NO: 32)
dna17 = 5′-CA GTG AGG CT GCA CCT GGA-3′
(SEQ ID NO: 33)
dna18 = 5′-TT GGG TTC A TT CCT CAC TG-3′
(SEQ ID NO: 34)
dna20 = 5′-/5Biosg/-AGAATGCTGAGATGTAGA-3′

### Synthesis of Phenanthroline-Alkyne

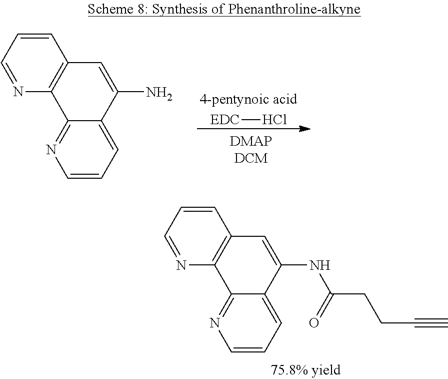

Phenanthroline-alkyne ligand was synthesized according to a procedure reported in the literature 3 using 4-pentynoic acid. 5-Amino-1,10-phenanthroline (150 mg, 0.77 mmol, 1 eq.) was dissolved in 6 mL of DCM. 4-Pentynoic acid (75 mg, 0.77 mmol, 1 eq) was then added to the mixture to create a dark orange gel. 1-ethyl-3-(3-dimethylaminopropyl)carbodiimide hydrochloride (EDCI, 295 mg, 1.54 mmol, 2.0 eq.) was then added followed by an additional 2 mL of DCM. Finally, 4-dimethylaminopyridine (DMAP, 94 mg, 0.77 mmol, 1 eq.) was added, and the reaction was stirred overnight at room temperature under N₂. The reaction mixture was concentrated under reduced pressure to afford a thick yellow oil. Water was added while stirring until a white precipitate was formed. The precipitate was isolated via centrifugation at 4300 rcf for 3 minutes. The supernatant was removed, and the powder was washed once with acetonitrile followed by centrifugation. The yellow solid (160 mg, 75.8%) was analyzed via NMR in CDCl₃.

¹H NMR (500 MHz, CDCl₃) δ 8.96 (s, 2H), 8.38 (dd, J=8.4, 1.6 Hz, 1H), 8.10-7.90 (m, 2H), 7.56-7.35 (m, 2H), 2.78 (m 2H), 2.66 (m 2H), 2.09 (s, 1H). ¹³C NMR (126 MHz, CDCl₃) δ 170.95, 149.87, 149.65, 146.22, 144.32, 135.89, 130.79, 130.63, 128.16, 124.50, 123.39, 122.62, 120.52, 83.05, 69.88, 35.90, 14.96.

Synthesis of Ru(bpy)₂(Phen-alkyne)Cl₂

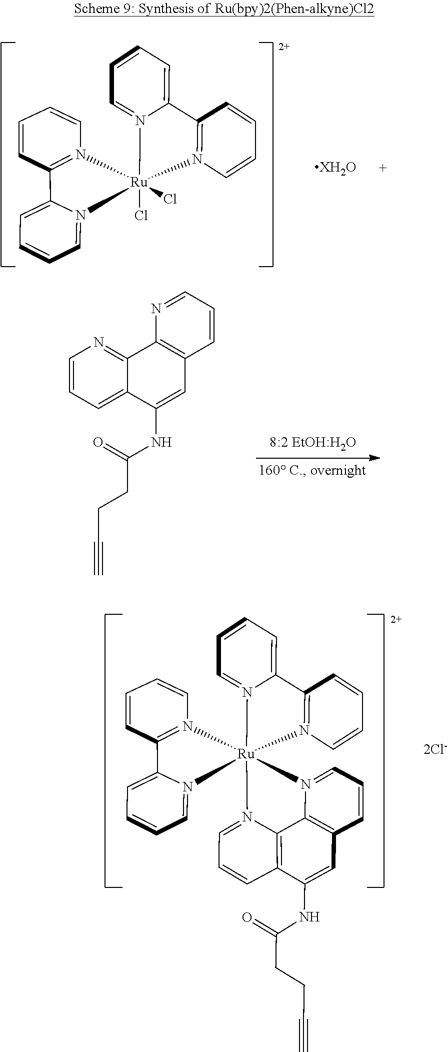

Phenanthroline-alkyne ligand (60 mg, 0.22 mmol, 1 eq.) was dissolved in 8 mL of ethanol and 2 mL of water. Cis-Ru(bpy)₂Cl₂·XH₂O (113.4 mg, 0.22 mmol, 1 eq.) was then added and the reaction was degassed with bubbling nitrogen for 15 minutes. The reaction then refluxed overnight at 160° C. while under nitrogen atmosphere. After cooling to room temperature, the reaction mixture was concentrated under reduced pressure. The crude product was purified using silica gel column chromatography, eluting with MeCN:H₂O:KNO₃ (100:7.5:0.5). The fractions containing product were combined and concentrated under reduced pressure. The solid product was then dissolved in MeCN, while the KNO₃ remained insoluble. The KNO₃ was removed via vacuum filtration to yield the Ru-alkyne complex as a red solid (21.2 mg, 0.028 mmol, 12.7%). ¹H and ¹³C NMR in MeOD. ESI-MS: expected 344.0743, observed 344.0742 m/z (+2 charge state).

¹H NMR (500 MHz, MeOD) δ 8.84 (dt, J=8.6, 1.7 Hz, 1H), 8.75 (d, J=8.2 Hz, 2H), 8.71 (d, J=8.2 Hz, 2H), 8.65 (dd, J=8.4, 1.3 Hz, 1H), 8.48 (d, J=7.4 Hz, 1H), 8.24 (dd, J=5.3, 1.2 Hz, 1H), 8.21-8.13 (m, 3H), 8.08 (td, J=7.9, 1.5 Hz, 2H), 7.95 (td, J=5.1, 1.4 Hz, 2H), 7.87 (dd, J=8.5, 5.2 Hz, 1H), 7.81 (dd, J=8.3, 5.2 Hz, 1H), 7.66 (d, J=5.6 Hz, 2H), 7.56 (ddt, J=7.5, 5.6, 1.7 Hz, 2H), 7.34 (ddt, J=7.3, 5.6, 1.5 Hz, 2H), 2.88 (t, J=7.0 Hz, 2H), 2.70 (dd, J=7.0, 2.6 Hz, 2H), 2.43 (s, 1H). ¹³C NMR (126 MHz, MeOD) δ 172.83, 172.72, 157.41, 157.39, 157.16, 157.14, 152.26, 151.51, 151.41, 151.38, 147.93, 145.79, 137.90, 137.78, 136.45, 134.06, 133.93, 132.86, 130.68, 127.63, 127.52, 127.49, 127.45, 127.42, 126.16, 125.57, 124.23, 124.21, 124.17, 124.15, 121.73, 82.27, 69.35, 35.05, 14.17.

### Ru(bpy)₂(Phen-Alkyne)—DNA Bioconjugation

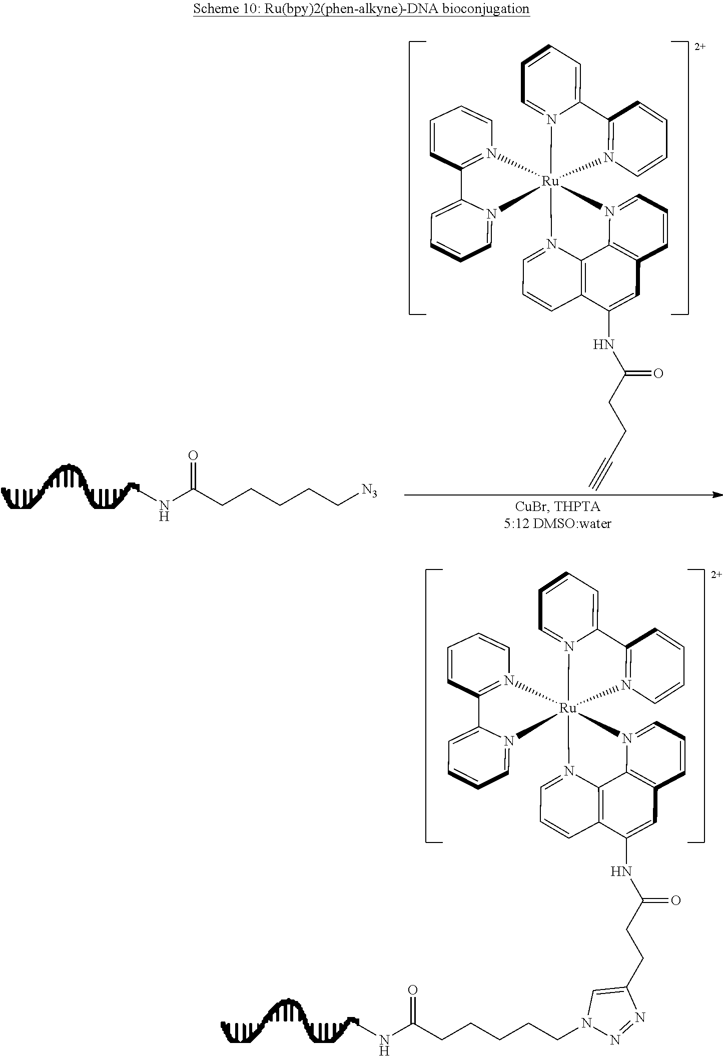

CuBr (50 mM, DMSO) was combined with tris-hydroxypropyltriazolylmethylamine (THPTA) (50 mM, DMSO) in a 1:4 ratio (1 μL of CuBr (50 mM) and 4 μL of THPTA (50 mM). To a 1.5 mL centrifuge tube, 1 nmol of azide modified DNA (100 μM, water) was added followed by 50 eq of CuBr complexed with THPTA and 10 eq of Ru(bpy)2(phen-alkyne) (5 mM, water). The sample was vortexed thoroughly, spun down in a bench-top centrifuge, and incubated at 37° C. for 1 hour with 300 rpm shaking while covered with foil to block light. Five identical crude reaction mixtures were combined for work-up using Amicon 3K ultra 0.5 mL devices, then diluted to 500 μL total volume with nuclease free water (NFW). Devices were spun for 8 minutes at 14,000 rcf 2 times, removing flow through and diluting to 500 μL with NFW each time. A third spin was performed for 15 minutes at 14,000 rcf. Samples were recovered by inverting the Amicon device into a clean tube and centrifuging for 2 minutes at 1,000 rcf. The conjugate was then purified by HPLC by injection of up to 5 nmol of DNA at a time. The fraction containing product was then buffer-exchanged using PBS, pH 7.4 with a second Amicon Ultra filtration device to remove residual triethyl ammonium acetate (TEAA) from the HPLC purification. Agarose gel electrophoresis was done to verify the complete removal of excess Ru-alkyne from the product. The concentration of Ru-DNA was calculated using UV-Vis absorbance at 260 and 450 nm. A standard curve of the A260 and A450 for the Ru-alkyne substrate was created (A260 extinction coefficient=21,800 cm⁻¹ M⁻¹, A450 extinction coefficient=10,800 cm⁻¹ M⁻¹), and DNA concentration was determined by subtracting the absorbance at 260 nm due to the Ru complex to find the DNA A260 contribution and using the extinction coefficient of DNA provided by IDT. To verify the identity of conjugates, intact mass analysis via electrospray mass spectrometry (ESI-MS) was performed on Ru-dna02 (see “Mass Spectrometry”). Az-dna02 had an observed mass of 6426.1902 and the Ru conjugate Ru-dna02 had an observed mass of 7113.3427. The difference of the observed masses was 687.1525 which corresponds to an m/z of 343.5763 of the +2 charge state (344.0742 m/z, +2 charge state observed for Ru(bpy)2(phen-alkyne)).

### Example 3: Reversibly-Switchable Photocatalysts for Radical Polymerization Using DNA and DNA Aptamer Scaffolds for Generalizable Stimuli

Using external stimuli to control catalysts for polymerization has garnered recent interest for its potential in additive manufacturing, patterning, and potential for controlling the microstructure of synthesized polymers. Modulating catalyst reactivity during synthesis could open routes to finely tuned microstructures, analogous to the control in bio-polymer syntheses, that are not feasible with traditional syntheses of co-polymers by timed addition of monomers and inherent monomer reactivities. In recent years, photo-controlled radical polymerization has especially garnered interest since light is an easily manipulated external stimuli, allowing spatio-temporal control. Other stimuli-responsive catalysts have used stimuli such as temperature, pH, chemical reagents, and redox state to control catalyst activity.

While there has been progress on expanding the list of stimuli ‘targets’, many of these catalysts involved significant rational design or require the target to directly modify the catalyst for activation. This leads to catalyst designs that are not easily adaptable to work with other stimuli. This is in contrast to biology which uses a broad array of stimuli including proteins, metal ions, small molecules such as hormones or nutrients (e.g. hydrocortisone, glucose), pH, etc that trigger responses or complex behavior. As a step towards bridging the gap between biological and synthetic catalysts, inventors envisioned that a hybrid biological-synthetic platform would be a promising strategy for creating a class of stimuli-responsive catalysts where the platform is readily adaptable to the types of stimuli that biological polymerization machinery responds to.

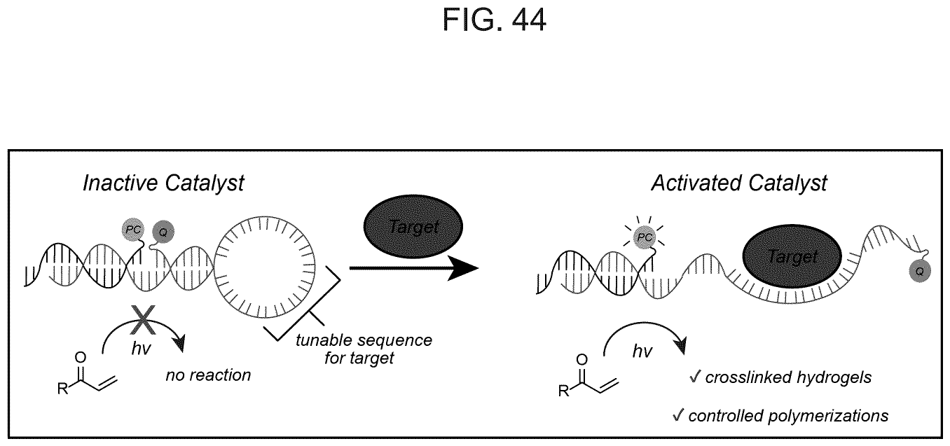

Specifically, inventors envisioned that switchable DNA scaffolded photocatalysts can be used as a generalizable platform for stimuli-responsive photocatalysts. These switchable catalysts use covalently modified DNA to control the proximity of a photocatalyst and a quencher dye. When these components are in proximity, Förster resonance energy transfer (FRET) or static quenching can occur where an excited photocatalyst transfers energy to the quencher (FRET acceptor) to undergo vibrational relaxation, preventing fluorescence or productive photocatalytic events. Controlling the proximity of the photocatalyst/quencher pair can then switch the photocatalyst between inactive and active states. FIG. 44 illustrates how DNA controls the proximity between the photocatalyst and quencher pair.

A benefit of these DNA scaffolded catalysts is that the responsive behavior originates from the DNA sequence and conformation, not the catalyst/photocatalyst itself. This allows for a potential “mix and match” where the catalyst of interest can be matched with the target specific DNA sequence. For nucleic acid type triggers (i.e. DNA and RNA strands), this scaffold follows the principles of molecular beacons. For non-nucleic acid type triggers, this scaffold takes advantage of aptamer sensors. Aptamers are sequences of nucleic acids that fold into specific conformations that allow binding of targets. Aptamers are capable of targeting a wide range of analytes from simple ions to small molecules to entire proteins. While there are already thousands of published sequences of aptamers, 12 new sequences for novel targets can be found through a process called systematic evolution of ligands by exponential enrichment (SELEX). Some of the most common approaches for sensor development from aptamers already involve the attachment of a fluorophore to the target binding DNA sequence and the partial hybridization of another DNA oligomer modified with a quencher dye. The partial hybridization brings the quencher dye into proximity of the fluorophore, allowing FRET to occur. Upon binding of the target, a conformational change in the DNA secondary structure disrupts the partial hybridization and removes the proximity of the fluorophore/quencher pair. Aptamer sensors can be developed to incorporate fluorophore/quencher pairs directly as part of the SELEX discovery process or by modifying existing aptamer sequences. These fluorescent sensors have been used for the detection of analytes and as probes in biological systems, but reports using aptamer sensors as switchable photocatalysts are primarily limited to the production of reactive oxygen species.

As an initial demonstration, inventors explored the bulk formation of a hydrogel through uncontrolled radical polymerization. This offered a sensitive detection method with an easily identified macroscopic output. Inventors explored reported aptamer sensors that targeted a range of analytes from DNA to ions to small molecules. A wide range of analyte types were chosen to demonstrate the generalizability of the chemical stimuli for this approach. Fluorescein modified DNA aptamers were commercially ordered since this was the most commonly used fluorophore in these systems. While fluorescein is predominately used as a fluorophore, it can act as a photocatalyst, albeit less potent than structurally similar photocatalysts such as Eosin Y.

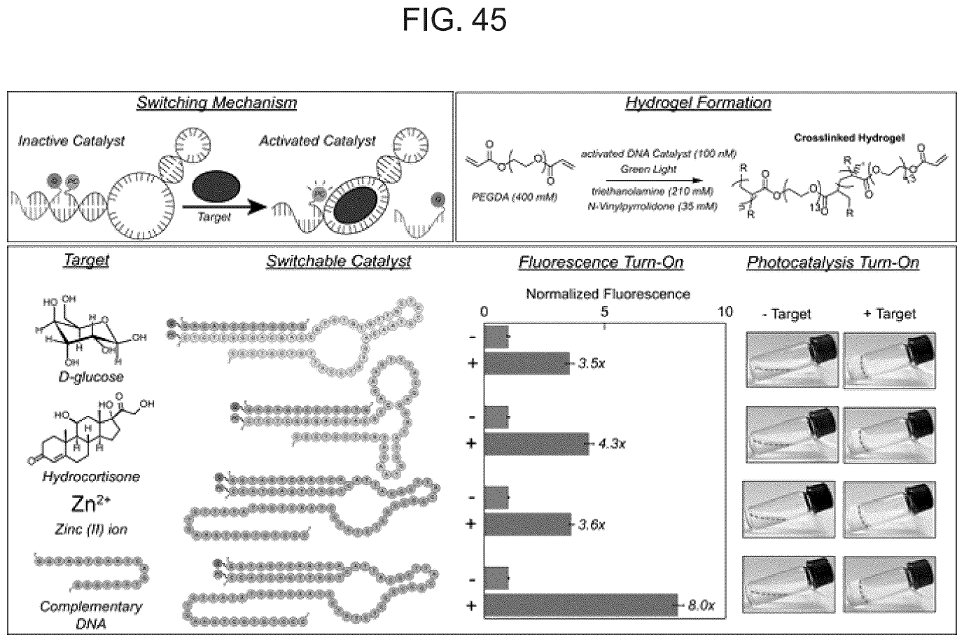

DNA aptamer sensors were dissolved in buffer and a reaction solution containing 25% v/v poly-ethylene glycol (Mn-700) diacrylate (PEGDA), 210 mM triethanolamine, and 35 mM N-Vinylpyrrolidone. The kinetics of DNA conformational switching were rapid and similar for the reaction conditions and fully aqueous buffer conditions. FIG. 45 (Fluorescence Turn-On) shows the fluorescence response for these DNA sensors at 100 nM concentration, normalized to the quenched/inactive state. Each system showed at least a 3.5× turn-on of fluorescence upon addition of target with the maximum response from complementary DNA-trigger. After five minutes of inert gas purging, these solutions were illuminated for one to two minutes with a green LED Wisconsin photoreactor. Samples without target remained as a liquid solution while samples with target formed a solid hydrogel (FIG. 45: Photocatalysis Turn-On).

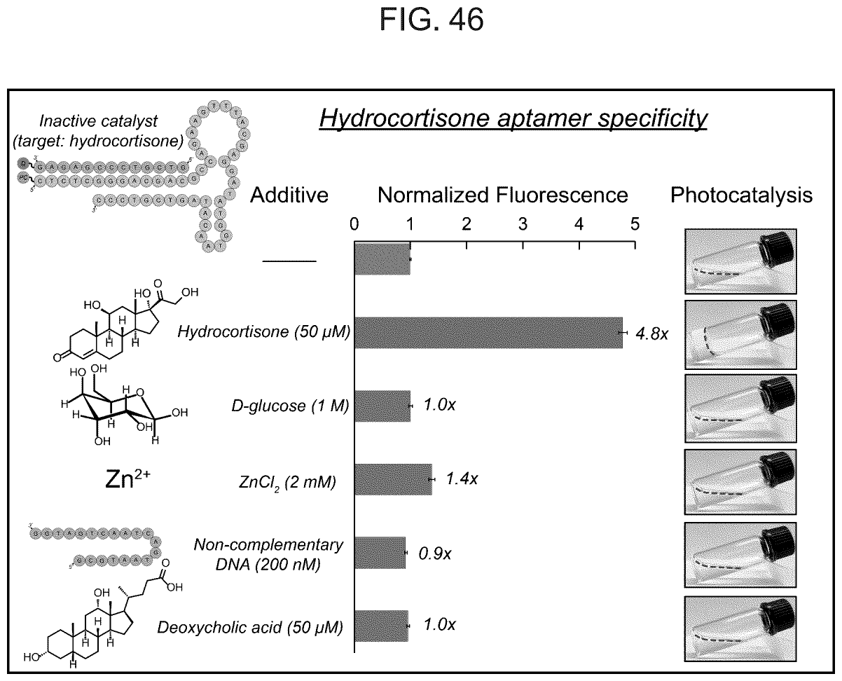

To demonstrate the specificity for the desired target, the switchable DNA catalyst for hydrocortisone was tested against the presence of all other targets and a structurally similar steroid, deoxycholic acid. FIG. 46 shows that only the addition of hydrocortisone caused a large response in fluorescence and the formation of a bulk hydrogel.

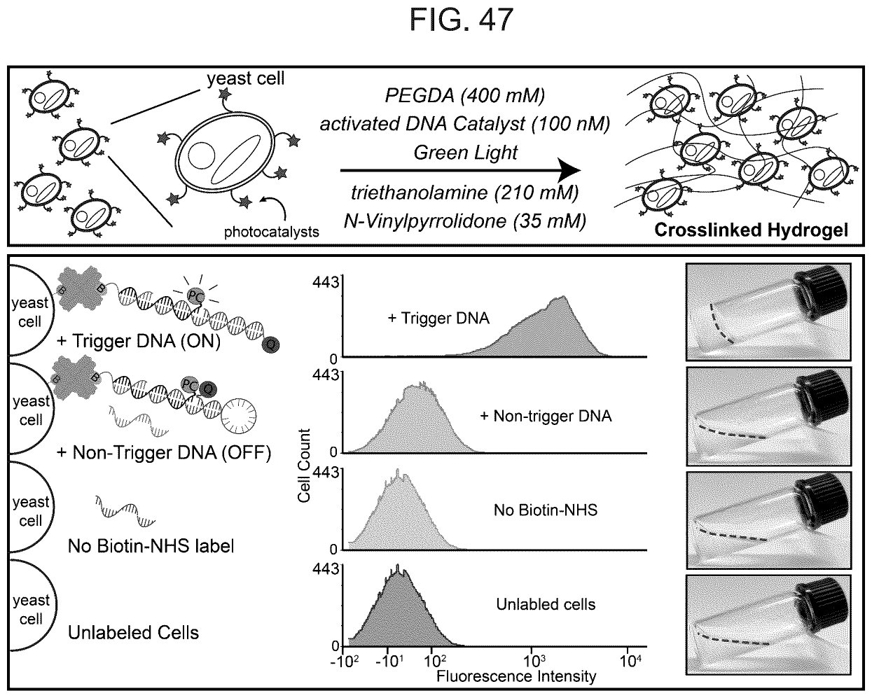

After validating photocatalytic turn-on using DNA aptamers and a range of target analytes, inventors aimed to demonstrate switchable polymerization activity on living cells. Polymer coatings on the surface of living cells have potential applications such as cell encapsulation/capture, functional cell coatings, and labeling through signal amplification. Inventors anchored a DNA ‘molecular hook’ scaffold to yeast cells through covalent labeling of nucleophilic residues using NHS-biotin followed by a streptavidin-biotin sandwich assay with a biotin modified DNA anchor strand (see FIG. 47 (left)). Fluorophore/photocatalyst modified DNA and quencher dye modified DNA were hybridized to the anchor strand to attach the DNA scaffolded photocatalyst to the cell surface. Flow cytometry was used to measure the fluorescence of individual yeast cells using a bright fluorophore, Cy3. FIG. 47 (middle) shows little to no fluorescence for cells without the scaffold, cells labeled with the “quenched” scaffold, and cells labeled with “quenched” scaffold in the presence of a non-binding DNA target. Strong fluorescent signal is shown for yeast cells with the DNA scaffold in the presence of DNA trigger.

The same constructs were made but with fluorescein photocatalyst instead of Cy5 fluorophore. Cells labeled with the fluorescein scaffolds were suspended in a similar solution of PEGDA monomer to test for bulk hydrogel formation. FIG. 47 (right) shows photocatalysis was turned on only by addition of trigger-DNA, resulting in bulk hydrogel formation. This demonstration shows that these DNA scaffolded catalysts can be attached to the surface of cells and triggered to turn on fluorescence or photocatalysis.

While the uncontrolled radical polymerizations demonstrated above offer a sensitive detection method, controlled radical polymerization techniques, such as reversible addition fragmentation chain-transfer (RAFT) polymerization, can be used for the synthesis of complex polymer architectures (e.g. block copolymers) and for modifying the surfaces of materials or even living cells. RAFT controls the chain growth of polymers through a reversible-deactivation process using chain transfer agents. This allows for targeted molecular weights, predictable growth kinetics, relatively narrow molecular weight distributions, and high end-group fidelity to be achieved. Combining photochemistry with RAFT, photoinduced electron/energy transfer-RAFT (PET-RAFT) is a type of photocontrolled polymerization that allows spatio-temporal control of polymer synthesis. Recent advances in PET-RAFT have demonstrated compatibility with aqueous conditions, ambient oxygen, biomolecules such as proteins and DNA, and even live cell surfaces. While PET-RAFT and similar photo-controlled polymerizations have myriad applications, there has been little work exploring other stimuli or dual-gated systems beyond light and pH or temperature.

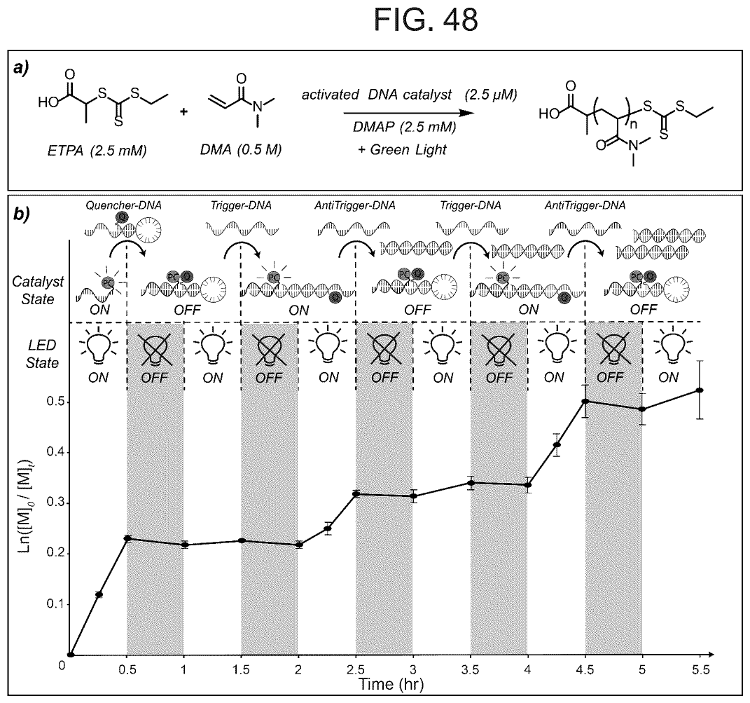

Inventors used the hybridization of DNA to reversibly activate and deactivate a photocatalyst during a PET-RAFT polymerization (FIG. 48a). FIG. 48b (top) illustrates the molecular hook design inventors used to bring a photocatalyst and quencher pair into proximity to quench both fluorescence and photocatalysis. The hairpin DNA structure of the quencher strand can be opened upon introduction and hybridization of a trigger DNA strand, removing the proximity of the photocatalyst and quencher pair to reactivate the photocatalyst. Inventors programmed the trigger strand to include an overhang region of eight base pairs so that a fully complementary (anti-trigger) strand can displace the trigger strand through toe-hold displacement. This brings the system back into the quenched state with a slight excess of spectator DNA. These last steps can be repeated to deactivate/reactivate the photocatalyst, with each cycle adding more spectator DNA into solution.

FIG. 48b (bottom) shows the pseudo-first order kinetics of polymerization with respect to N,N-dimethylacrylamide (DMA) concentration. Inventors demonstrated that our system required both illumination and the photocatalyst to be in an activated state for monomer consumption to occur. The shaded regions demonstrate when there was no light, resulting in no monomer conversion. When the photocatalyst was in the inactivated state (quenched or after addition of anti-trigger) little to no polymerization occurred, even when light was present. Size exclusion chromatography (SEC) showed dispersity values between 1.1-1.25, demonstrating a relatively narrow molecular weight distribution was maintained. A plot of Mn, SEC vs monomer conversion showed a linear relationship as expected for a controlled radical polymerization. This demonstration showed that a photocontrolled polymerization can be reversibly activated by a conformational change due to DNA hybridization.

In summary, inventors demonstrated a modular platform to generate switchable photocatalysts for polymerizations using a DNA scaffolded system that depends on the DNA sequence and conformational state to control catalysis rather than the inherent properties of the catalyst. Inventors expect that DNA aptamer sensors modified with potent photocatalysts can be used as a dual-gated system for photocontrolled polymerization where the biological/chemical signal is as generalizable as aptamer targets. Inventors expect that these switchable photocatalysts can be designed for complex responses such as the growth of polymer coatings on whole or sub-regions of cells for specific chemical targets. The union of photocatalysis with DNA scaffolds and aptamers demonstrates a near limitless number of chemical stimuli can be used to tune the activation of catalysis, taking one step closer to mimicking the complicated signaling that is important in biological systems. Future studies will be aimed towards applications of this system on the surface of living cells for creating unique surface modifications and coatings.

What is claimed:

1. A nucleic acid-scaffolded catalytic system, comprising:
a nucleic acid catalyst including a first nucleic acid strand and, optionally, a second nucleic acid strand, a first reactive moiety attached to the first nucleic acid strand, and a second moiety attached to the first nucleic acid strand or the second nucleic acid strand, wherein a catalytic activity of the first reactive moiety is dependent on a distance between the first reactive moiety and the second moiety.

2. The nucleic acid-scaffolded catalytic system of claim 1, wherein binding of a trigger to the nucleic acid structure triggers an increase in the catalytic activity of the first reactive moiety.

3. The nucleic acid-scaffolded catalytic system of claim 1, wherein binding of a trigger to the nucleic acid structure triggers a decrease in the catalytic activity of the first reactive moiety.

4. The nucleic acid-scaffolded catalytic system of claim 1, wherein the first reactive moiety and the second moiety are abiotic groups.

5. The nucleic acid-scaffolded catalytic system of claim 1, wherein:
the nucleic acid scaffold includes the first nucleic acid strand folded into a stem-loop structure;
the first reactive moiety is attached to a first end of the first nucleic acid strand, and the second moiety is attached to a second end of the first nucleic acid strand such that the first reactive moiety and the second moiety are in proximity in the stem-loop structure; and
when a trigger that is a template nucleic acid strand anneals with the first nucleic acid, the stem-loop structure opens to move the second moiety farther away from the first reactive moiety.

6. The nucleic acid-scaffolded catalytic system of claim 1, wherein:
the first reactive moiety is attached to the first nucleic acid strand;
the second moiety is attached to the second nucleic acid strand; and
when a trigger that is a template nucleic acid strand anneals with the first nucleic acid strand and the second nucleic acid strand, the first reactive moiety is brought into proximity to the second moiety.

7. The nucleic acid-scaffolded catalytic system of claim 1 further comprising a trigger that binds to either or both of the first nucleic acid strand and the second nucleic acid strand to trigger an increase or a decrease in the catalytic activity of the first reactive moiety by altering the distance between the first reactive moiety and the second moiety.

8. The nucleic acid-scaffolded catalytic system of claim 7, wherein the trigger is selected from a group consisting of an ion, a small molecule, a protein, and a nucleic acid.

9. The nucleic acid-scaffolded catalytic system of claim 7, wherein the analyte is single stranded DNA or an RNA oligomer that anneals with the first nucleic acid strand or to both of the first nucleic acid strand and the second nucleic acid strand.

10. The nucleic acid-scaffolded catalytic system of claim 1 further comprising
a first split invader construct comprising a first protein binding moiety linked with a nucleic acid strand having a first complementary domain and a first complementary catalyst domain opposite the first protein binding domain; and
a second split invader construct comprising a second protein binding moiety linked with a nucleic acid strand having a second complementary domain and a second complementary catalyst domain opposite the second protein binding moiety;
wherein the first protein binding moiety and the second protein binding moiety bind to sites on one or more proteins;
wherein the first complementary domain and the second complementary domain are complementary to one another and when the first complementary domain and second complementary domain are annealed with each other, the first split invader construct and second split invader construct form a full invader duplex, and
wherein the full invader duplex can trigger the nucleic acid-scaffolded catalytic system by binding the first complementary catalyst domain and the second complementary catalyst domain of the full invader duplex to either or both of the first nucleic acid strand and the second nucleic acid strand to trigger an increase or a decrease in the catalytic activity of the first reactive moiety by altering the distance between the first reactive moiety and the second moiety.

11. The nucleic acid-scaffolded catalytic system of claim 1, wherein either or both of the first protein binding moiety and the second protein binding moiety is an aptamer or an antibody.

12. The nucleic acid-scaffolded catalytic system of claim 1, wherein the first reactive moiety is a photocatalyst.

13. The nucleic acid-scaffolded catalytic system of claim 1, wherein the first reactive moiety catalyzes the production of a colored or fluorescent product.

14. The nucleic acid-scaffolded catalytic system of claim 1, wherein the first reactive moiety catalyzes conversion of a prodrug to an efficacious drug.

15. The nucleic acid-scaffolded catalytic system of claim 1, wherein the first reactive moiety catalyzes the formation of a polymer.

16. A method for controlling a rate of a reaction with a nucleic acid-scaffolded catalyst, including: triggering a conformational shift in the nucleic acid-scaffolded system according to claim 1 by addition of a trigger that binds to the nucleic acid scaffold, the conformational shift in the nucleic acid scaffold changing the rate of the reaction by changing the distance between the first reactive moiety and the second moiety.

17. The method of claim 16, wherein the first reactive moiety is a photocatalyst and the method further comprises irradiating the photocatalyst.

18. A method for detecting an analyte, including:
exposing the nucleic acid scaffold system according to claim 1 to a sample, wherein if the sample contains the analyte, then the analyte binds to the nucleic acid scaffold and triggers a conformational shift in the nucleic acid scaffold that changes the distance between the first reactive moiety and the second moiety and the catalytic activity of the first reactive moiety; and
detecting the presence of the analyte based on the change in catalytic activity of the first reactive moiety.

19. A method for detecting a protein-protein interaction, including:
exposing the nucleic acid-scaffolded system according to claim 10 to a sample comprising the one or more proteins; wherein trimolecular annealing between the first complementary catalyst domain, the second complementary catalyst domain, and either or both of the first nucleic acid strand and the second nucleic acid strand triggers a conformational shift that changes the distance between the first reactive moiety and the second moiety; and
detecting the trimolecular annealing based on the change in catalytic activity of the first reactive moiety.

20. The method of claim 19, wherein photocatalytic labeling is detected.

21. The method of claim 19, wherein fluorescence is detected.

22. The method of claim 19, wherein the one or more proteins are on one or more cell surfaces.
